(19) United States
(12) Patent Application Publication
(10) Pub. No.: US 2025/0296920 A1
(43) Pub. Date: Sep. 25, 2025

(54) FORMULATIONS COMPRISING N-SUBSTITUTED-DIOXOCYCLOBUTENYLAMINO-3-HYDROXY-PICOLINAMIDE COMPOUNDS AND USES THEREOF

(71) Applicant: Pfizer Inc., New York, NY (US)

(72) Inventors: Catherine Michelle Ambler, Gales Ferry, CT (US); Kapildev Kashmirilal Arora, East Lyme, CT (US); Samir Kulkarni, Carmel, IN (US); Duangporn Wanapun, Mystic, CT (US)

(73) Assignee: Pfizer Inc., New York, NY (US)

(21) Appl. No.: 19/084,960

(22) Filed: Mar. 20, 2025

Related U.S. Application Data
(60) Provisional application No. 63/567,990, filed on Mar. 21, 2024.

(51) Int. Cl. C07D 401/12; A61K 31/4439; A61K 31/5377; A61K 31/675; C07D 405/14; C07D 413/12; C07D 417/12; C07D 417/14; C07F 9/6503

(52) U.S. Cl. CPC C07D 401/12; A61K 31/4439; A61K 31/5377; A61K 31/675; C07D 405/14; C07D 413/12; C07D 417/12; C07D 417/14; C07F 9/65031; C07B 2200/07

(57) ABSTRACT

The present disclosure relates to compositions comprising N-substituted-dioxocyclobutenylamino-3-hydroxy-picoliamide compounds or a pharmaceutically acceptable salt or hydrate thereof, methods of preparing said compositions, and uses thereof. The compositions may be useful in the treatment of conditions ameliorated by inhibition of CC chemokine receptor 6.

### BACKGROUND

The CC chemokine receptor 6 (CCR6) is a G protein-coupled receptor expressed on a variety of key immune cells including immature dendritic cells, B cells, memory T cells, neutrophils, and a subset of Tregs. The ligand-receptor pair CCL20-CCR6 is responsible for the migration of immature dendritic cells and effector/memory T-cells to the skin and mucosal surfaces under homeostatic and inflammatory conditions, as well as in autoimmune diseases such as psoriasis and inflammatory bowel diseases. Therapeutic agents that act as CCR6 inhibitors and pharmaceutical compositions comprising CCR6 inhibitors may be effective in treating inflammatory diseases.

### SUMMARY OF THE INVENTION

The present invention provides, in part, a composition that can inhibit the activity of CCR6 and may be useful in the treatment of a condition. In one embodiment, described herein is a composition comprising: a) a compound of Formula (IA) or Formula (IB):

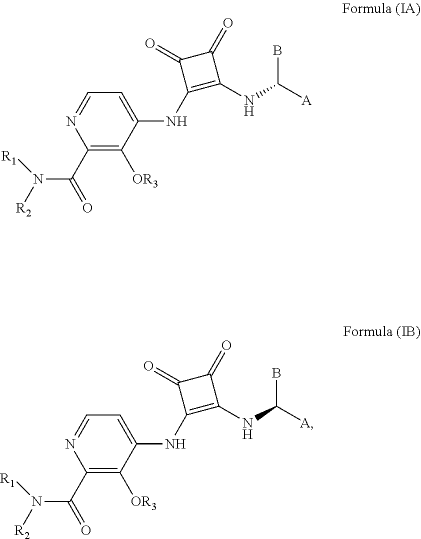

or a pharmaceutically acceptable salt or hydrate thereof, wherein:
R₁ and R₂ are independently H, (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of 0, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is

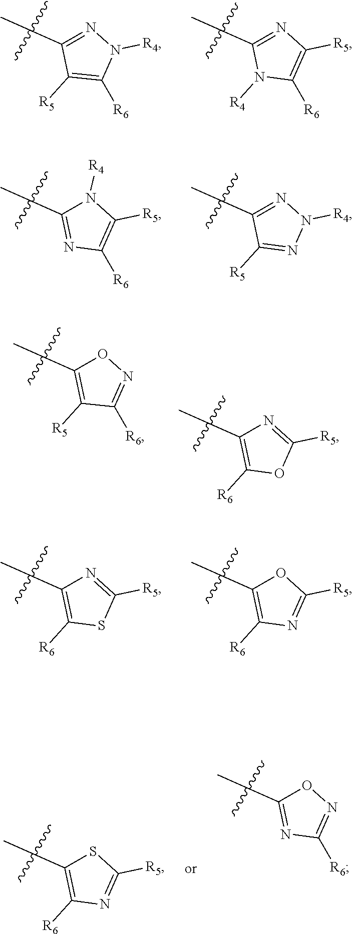

R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁₋₉, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is

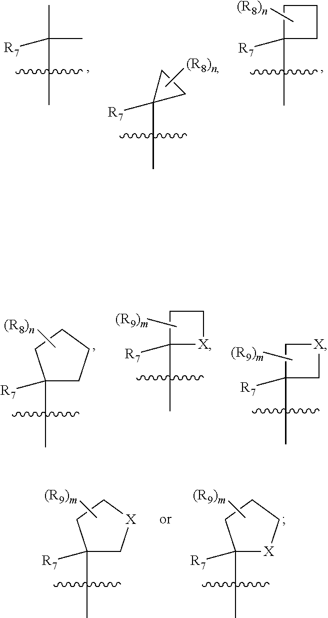

R₇ is —F, —CN, (C₁-C₃)alkyl, (C₁-C₃)alkyl-d₁₋₇, or halo(C₁-C₃)alkyl;
R₃ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₉ at each occurrence is independently deuterium, —F, —Cl, —Br, or —I, or two R₉s, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl; and
b) a polymer,
wherein the compound and the polymer are in an amorphous solid dispersion.

In one embodiment, described herein is a method of treating a condition comprising administering to a subject in need thereof a pharmaceutical composition comprising:
a) a therapeutically effective amount of a compound of Formula (IA) or Formula (IB):

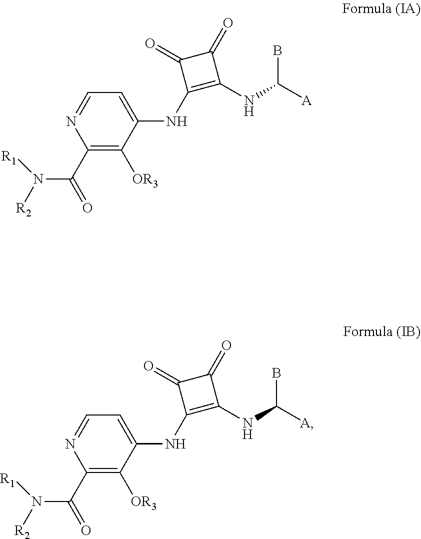

or a pharmaceutically acceptable salt or hydrate thereof, wherein:
R₁ and R₂ are independently H, (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is

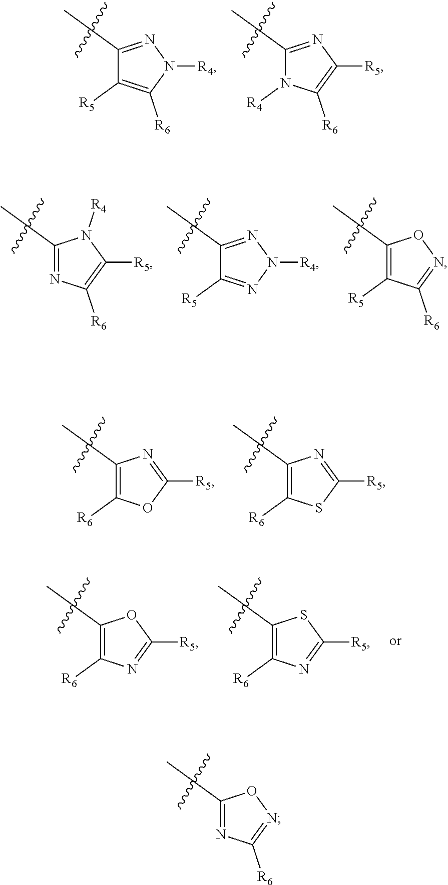

R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁-9, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is

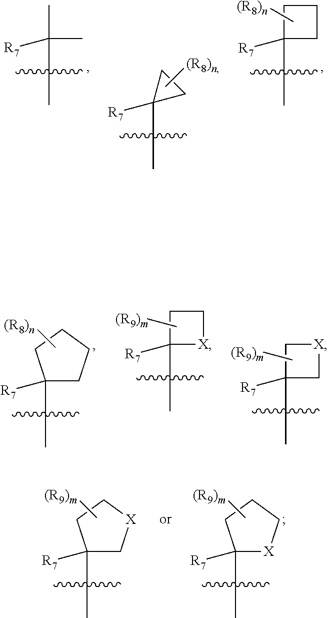

R₇ is —F, —CN, (C₁-C₃)alkyl, (C₁-C₃)alkyl-d₁₋₇, or halo(C₁-C₃)alkyl;
R₈ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₉ at each occurrence is independently deuterium, —F, —Cl, —Br, or —I, or two R₉s, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl; and
b) a polymer,
   wherein the compound and the polymer are in an amorphous solid dispersion, wherein the condition is selected from the group consisting of: psoriasis, rheumatoid arthritis, juvenile arthritis, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile idiopathic arthritis (JIA), juvenile psoriatic arthritis, juvenile rheumatoid arthritis, systemic onset juvenile rheumatoid arthritis, giant cell arteritis, secondary osteoarthritis from inflammatory disease, inflammatory bowel disease, Crohn's disease, and ulcerative colitis.

It is to be understood that both the foregoing general description and the following detailed description are exemplary and explanatory only and are not restrictive of the invention, as claimed.

### BRIEF DESCRIPTIONS OF THE DRAWINGS

FIG. 1 illustrates the process of the co-precipitation process of an active pharmaceutical ingredient (API).

FIG. 2A shows an image of the spray nozzle used to prepare a co-precipitation formulation of Compound A (KL1 batch). FIG. 2B and FIG. 2C show images of the co-precipitated material, which was porous with a very high surface area.

FIG. 3A shows an image of the air-assisted spray nozzle used to prepare a co-precipitation formulation of Compound A (KL2 batch). FIG. 3B and FIG. 3C show images of the co-precipitated material, which had a desired porosity and low surface area.

FIG. 4A shows powder X-ray diffraction data obtained for a co-precipitated formulation with Compound A. FIG. 4B shows thermogravimetric analysis data obtained for a co-precipitated formulation with Compound A.

FIG. 5A shows modulated differential scanning calorimetry data obtained for a co-precipitated formulation with Compound A. FIG. 5B shows dynamic vapor sorption data obtained for a co-precipitated formulation with Compound A.

FIG. 6 shows the co-precipitation properties of the material.

FIG. 7A compares the tensile strength of three different amorphous solid dispersion materials: co-precipitation (KL1), co-precipitation (KL2), and SDD. FIG. 7B compares the Brunauer-Emmett-Teller (BET) surface areas of three different amorphous solid dispersion materials: co-precipitation (KL1), co-precipitation (KL2), and SDD. FIG. 7C shows flow function coefficient measurements for three different solid dispersion materials: co-precipitation (KL1), co-precipitation (KL2), and SDD. FIG. 7D shows bulk density measurements of three different amorphous solid dispersion materials: co-precipitation (KL1), co-precipitation (KL2), and SDD.

FIG. 8A shows the roller compaction profile of the immediate release tablet prepared using co-precipitated compound A. FIG. 8B shows the tabletability profile of the immediate release tablet prepared using co-precipitated compound A. FIG. 8C shows the compactability (tensile strength) profile of the immediate release tablet prepared using co-precipitated compound A.

FIG. 9 shows the brittle fracture index of three different amorphous solid dispersion materials: co-precipitation (KL1), co-precipitation (KL2), and SDD.

FIG. 10 shows microscopic images of compressed tablets comprising material made with three different amorphous solid dispersion materials: co-precipitation (KL1), co-precipitation (KL2), and SDD.

FIG. 11 shows the dissolution profiles of Compound A, a Co-PPT formulation with compound A, Co-PPT tablet, SDD, and a SDD tablet.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

### Definitions

Unless otherwise defined herein, scientific and technical terms used in connection with the present invention have the meanings that are commonly understood by those of ordinary skill in the art. The invention described herein suitably may be practiced in the absence of any element(s) not specifically disclosed herein. “Compounds of the invention” include compounds of Formula (IA), (IB), (IIA), (IIB), (IIIA), or (IIIB), or a pharmaceutically acceptable salt or hydrate thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof (including deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter means that the parameter may vary by as much as 10% below or above the stated numerical value for that parameter. For example, a dose of about 5 mg means 5%±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

The term “carbonyl” as used herein, means a —C(═O)— group.

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Haloalkyl” refers to an alkyl group as defined above containing the specified number of carbon atoms wherein at least one hydrogen atom has been replaced by halogen. Haloalkyl groups man contain, but are not limited to, 1-6 carbon atoms (“C₁-C₆ haloalkyl”), 1-4 carbon atoms (“C₁-C₄ haloalkyl”), or 1-2 carbon atoms (“C₁-C₂ haloalkyl”). More specifically, fluorinated alkyl groups may be specifically referred to as “fluoroalkyl.”

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Haloalkoxy” refers to an alkoxyl group as defined above containing the specified number of carbon atoms wherein at least one hydrogen atom has been replaced by halogen. Haloalkoxy groups may contain, but are not limited to, 1-6 carbon atoms, (“C₁-C₆ haloalkoxy”), 1-4 carbon atoms (“C₁-C₄ haloalkoxy”), or 1-2 carbon atoms (“C₁-C₂ haloalkoxy”). More specifically, fluorinated alkoxyl groups may be specifically referred to as “fluoroalkoxy.”

“Alkoxyalkyl” refers to an alkyl group, as defined herein, that is substituted by an alkoxy group, as defined herein. Examples include, but are not limited to, CH₃OCH₂— and CH₃CH₂OCH₂—.

“Alkenyl” refers to an alkyl group, as defined herein, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Examples include, but are not limited to, ethynyl, 1-propynyl, 2-propynyl, 1-, 2-, or 3-butynyl, and the like.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O—. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”). Cycloalkoxy groups include, but are not limited to, cyclopropoxy, cyclobutoxy, cyclopentoxy and the like.

The term “(C₂-C₄)alkenyl” as used herein, means a straight or branched chain hydrocarbon containing from 2 to 4 carbons and containing one carbon-carbon double bond. Representative examples of (C₂-C₄)alkenyl include, but are not limited to, ethenyl, 2-propenyl, 2-methyl-2-propenyl, and 3-butenyl.

The term “(C₁-C₄)alkoxy” as used herein, means a (C₁-C₄)alkyl group, as defined herein, appended to the parent molecular moiety through an oxygen atom. Representative examples of (C₁-C₄)alkoxy include, but are not limited to, methoxy, ethoxy, propoxy, 2-propoxy, butoxy, and tert-butoxy.

The term “(C₁-C₃)alkyl” as used herein, means a straight or branched chain hydrocarbon containing from 1 to 3 carbon atoms. Representative examples of (C₁-C₃)alkyl include methyl, ethyl, n-propyl, and iso-propyl.

The term “(C₁-C₃)alkyl-d₁₋₇” as used herein, means a straight or branched chain hydrocarbon containing from 1 to 3 carbon atoms wherein one to seven of the hydrogens have been exchanged for deuterium (²H or D). Representative examples of (C₁-C₃)alkyl-d_{1.7} include methyl-d₃, ethyl-d₅, and ethyl-2,2,2-d₃.

The term “(C₁-C₄)alkyl” as used herein, means a straight or branched chain hydrocarbon containing from 1 to 4 carbon atoms. Representative examples of (C₁-C₄)alkyl include methyl, ethyl, n-propyl, iso-propyl, n-butyl, sec-butyl, iso-butyl, and tert-butyl.

The term “(C₁-C₄)alkyl-d₁₋₉” as used herein, means a straight or branched chain hydrocarbon containing from 1 to 4 carbon atoms wherein one to nine of the hydrogens have been exchanged for deuterium (²H or D). Representative examples of (C₁-C₄)alkyl-d₁₋₉ include methyl-d₃, ethyl-d₅, and ethyl-2,2,2-d₃.

The term “(C₁-C₆)alkyl” as used herein, means a straight or branched chain hydrocarbon containing from 1 to 6 carbon atoms. Representative examples of (C₁-C₆)alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “(C₁-C₄)alkylcarbonyl” as used herein, means a (C₁-C₄)alkyl group, as defined herein, appended to the parent molecular moiety through a carbonyl group, as defined herein. Representative examples of (C₁-C₄)alkylcarbonyl include, but are not limited to, acetyl, 1-oxopropyl, and 2,2-dimethyl-1-oxopropyl.

The term “(C₃-C₄)cycloalkyl” as used herein, means a saturated cyclic hydrocarbon group containing from 3 to 4 carbons, examples of (C₃-C₄)cycloalkyl include cyclopropyl and cyclobutyl. The (C₃-C₄)cycloalkyl groups of the invention are optionally substituted with 1, 2, or 3 substituents that are independently (C₁-C₄)alkyl, CN, halogen, or OH.

The term “(C₃-C₄)cycloalkyl(C₁-C₄)alkyl” as used herein, means a (C₃-C₄)cycloalkyl as defined herein, appended to the parent molecular moiety through a (C₁-C₄)alkyl group, as defined herein. Representative examples of (C₃-C₄)cycloalkyl(C₁-C₄)alkyl include cyclopropyl methyl, 2-cyclopropylethyl, 2-cyclopropylpropyl, 3-cyclopropylpropyl, cyclobutylmethyl, 2-cyclobutylethyl, 2-cyclobutylpropyl, and 3-cyclobutylpropyl.

The term “(C₃-C₅)cycloalkyl” as used herein, means a saturated cyclic hydrocarbon group containing from 3 to 5 carbons, examples of (C₃-C₅)cycloalkyl include cyclopropyl, cyclobutyl, and cyclopentyl. The (C₃-C₅)cycloalkyl groups of the invention are optionally substituted with 1, 2, or 3 substituents that are independently (C₁-C₄)alkyl, CN, halogen, or OH.

The term “halo” or “halogen” as used herein, means —Cl, —Br, —I or —F.

The term “halo(C₁-C₄)alkoxy” as used herein, means at least one halogen, as defined herein, appended to the parent molecular moiety through a (C₁-C₄)alkoxy group, as defined herein. Representative examples of halo(C₁-C₄)alkoxy include, but are not limited to, chloromethoxy, difluoromethoxy, 2-fluoroethoxy, trifluoromethoxy, and pentafluoroethoxy.

The term “halo(C₁-C₃)alkyl” as used herein, means at least one halogen, as defined herein, appended to the parent molecular moiety through a (C₁-C₃)alkyl group, as defined herein. Representative examples of halo(C₁-C₃)alkyl include, but are not limited to, chloromethyl, difluoromethyl, 2-fluoroethyl, trifluoromethyl, and pentafluoroethyl.

The term “halo(C₁-C₄)alkyl” as used herein, means at least one halogen, as defined herein, appended to the parent molecular moiety through a (C₁-C₄)alkyl group, as defined herein. Representative examples of halo(C₁-C₄)alkyl include, but are not limited to, chloromethyl, 2-fluoroethyl, trifluoromethyl, and pentafluoroethyl.

The term “hydroxy(C₁-C₄)alkyl” as used herein, means at least one hydroxy group, as defined herein, is appended to the parent molecular moiety through a (C₁-C₄)alkyl group, as defined herein. Representative examples of hydroxy(C₁-C₄)alkyl include, but are not limited to, hydroxymethyl, 2-hydroxyethyl, 3-hydroxypropyl, 2,4-dihydroxybutyl, and 2,3-dihydroxypropyl.

The term “heterocycle” or “heterocyclic” as used herein, means a 4, 5, or 6 membered ring containing one N heteroatom and optionally 1 or 2 additional heteroatoms selected from the group consisting of O, N, and S. Representative examples of heterocycle include, but are not limited to, azetidinyl, imidazolidinyl, morpholinyl, oxadiazolidinyl, oxazolidinyl, piperazinyl, piperidinyl, pyrazolidinyl, pyrrolidinyl, thiadiazolidinyl, thiazolidinyl, and thiomorpholinyl. The heterocycle groups of the invention are optionally substituted with 1, 2, or 3 (C₁-C₄)alkyl groups. “Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

“Amino” refers to a group —NH₂, which is unsubstituted. Where the amino is described as substituted or optionally substituted, the term includes groups of the form —NRxRy, where each of Rx and Ry is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

The term “pharmaceutically acceptable” means the substance (e.g., the compounds described herein) and any salt or hydrate thereof, or composition containing the substance or salt of the invention is suitable for administration to a subject or patient.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“Excipient” as used herein describes any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat” or “treatment” as used herein embraces both preventative, i.e., prophylactic, and palliative treatment, i.e., reverse, relieve, alleviate, or slow the progression of the patient's disease, disorder, or condition, or any tissue damage associated with the disease, disorder, or condition.

As used herein, the term, “subject, “individual” or “patient,” used interchangeably, refers to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

### Compounds of the Invention

In one embodiment, disclosed herein is a composition comprising a compound of Formula (IA) or Formula (IB):

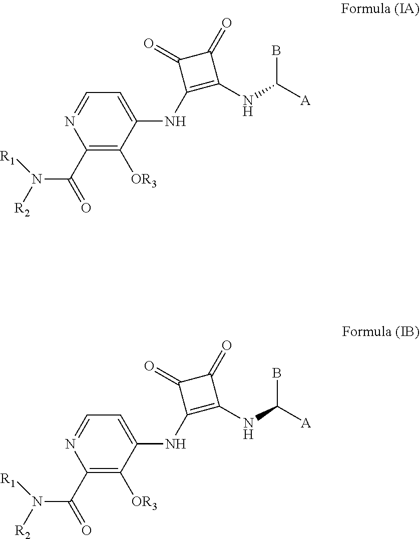

or a pharmaceutically acceptable salt or hydrate thereof, wherein
R₁ and R₂ are independently H or (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is

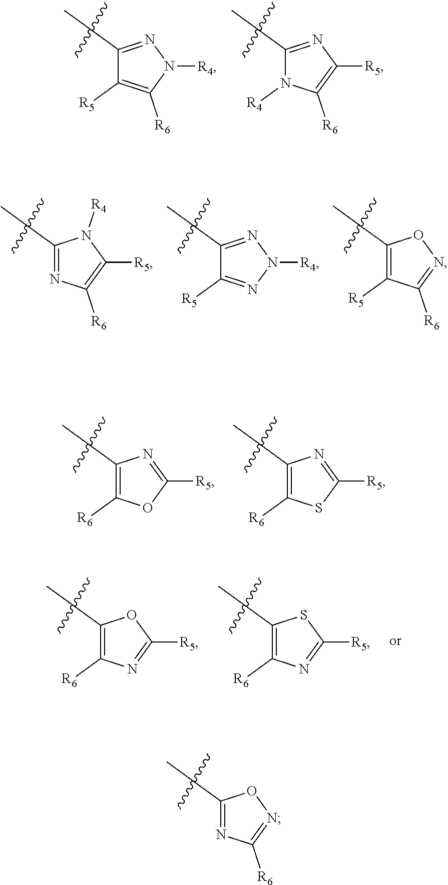

R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁₋₉, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is

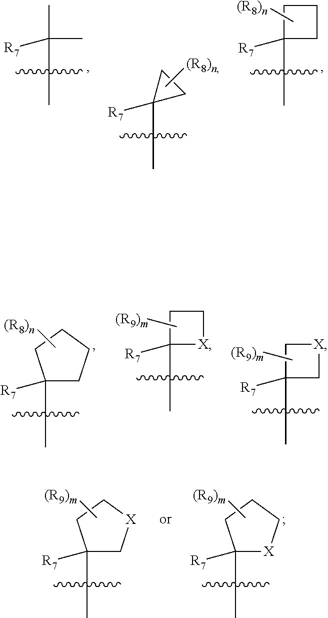

R₇ is —F, —CN, (C₁-C₃)alkyl, (C₁-C₃)alkyl-d₁₋₇, or halo(C₁-C₃)alkyl;
R₈ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₉ at each occurrence is independently deuterium, —F, —Cl, —Br, or —I, or two R₉s, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl.

In another embodiment, the present invention provides compositions comprising compounds of Formulae (IA) and (IB)

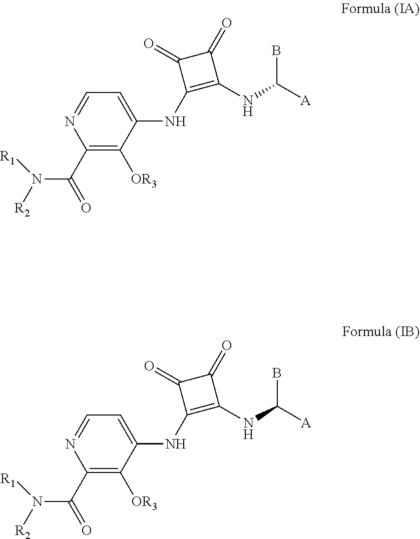

or a pharmaceutically acceptable salt or hydrate thereof, wherein R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl; R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl; A is

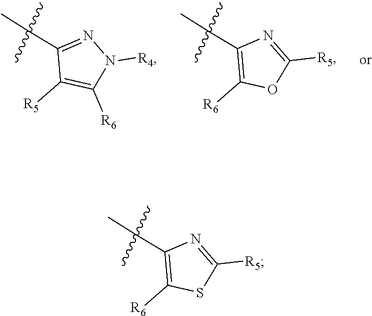

R₄ is (C₁-C₄)alkyl; R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, halo(C₁-C₄)alkyl, or hydroxy(C₁-C₄)alkyl; B is

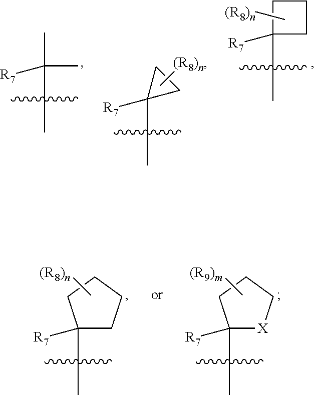

R₇ is (C₁-C₃)alkyl or C₁-C₃)alkyl-d₁₋₇; R₈ is deuterium or two R₈s, together with the same carbon atom to which the two R₈s are attached, form a (C₃-C₅)cycloalkyl group; n is 0 or 2; Rₛ at each occurrence is F; m is 0, 1, 2, 3, or 4; and X is O.

In another embodiment, the present invention provides compositions comprising compounds of Formula (IA) or (IB), or a pharmaceutically acceptable salt or hydrate thereof, wherein R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl; R₃ is H; A is

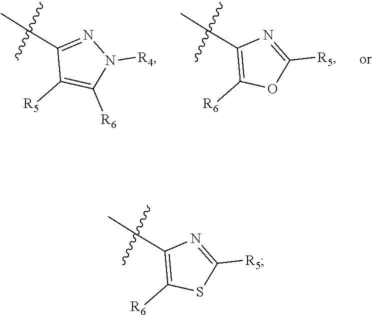

R₄ is (C₁-C₄)alkyl; R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, halo(C₁-C₄)alkyl, or hydroxy(C₁-C₄)alkyl; B is

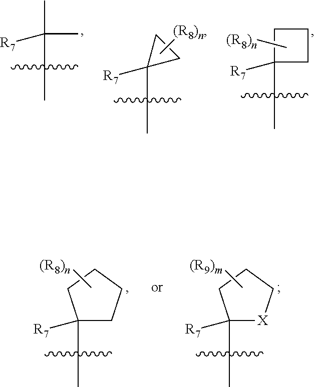

R₇ is (C₁-C₃)alkyl or C₁-C₃)alkyl-d₁₋₇, R₈ is deuterium or two R₈s, together with the same carbon atom to which the two R₈s are attached, form a (C₃-C₅)cycloalkyl group; n is 0 or 2; R₉ at each occurrence is F; m is 2; and X is O.

In another embodiment, the present invention provides compositions comprising compounds of Formula (IA) or (IB), or a pharmaceutically acceptable salt or hydrate thereof, wherein R₁ and R₂ are independently methyl, ethyl, or isopropyl, or R₁ and R₂ taken together with the nitrogen atom to which they are attached form morpholine or piperazine wherein the piperazine is substituted with methyl; R₃ is H; A is

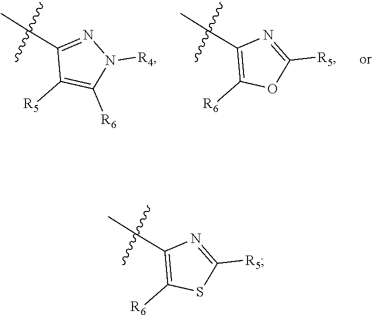

R₄ is methyl; Rₛ is methyl, methyl-d₃, ethyl, methoxy, Cl, difluoromethoxy, cyano, cyclopropyl, or hydroxymethyl; R₆ is H or methyl; B is

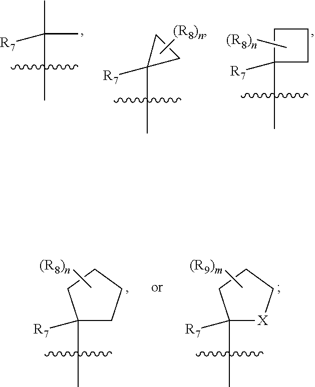

R₇ is methyl; R₈ is deuterium or two R₈s, together with the same carbon atom to which the two R₈s are attached, form cyclopropyl; n is 0 or 2; R₉ at each occurrence is F; m is 2; and X is O.

In another embodiment, the present invention provides compositions comprising compounds of Formula (IA) or (IB), or a pharmaceutically acceptable salt or hydrate thereof, wherein R₁ is methyl; R₂ is methyl, ethyl, or isopropyl; R₃ is H; A is

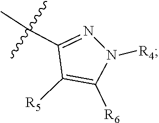

R₄ is methyl; Rₛ is methyl, methyl-d₃, ethyl, methoxy, Cl, difluoromethoxy, cyano, or cyclopropyl; R₆ is H; B is

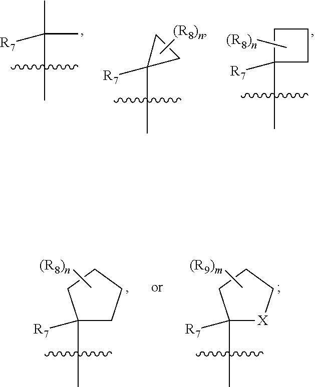

R₇ is methyl; R₃ is deuterium or two R₈s, together with the same carbon atom to which the two R₈s are attached, form cyclopropyl; n is 0 or 2; R₉ at each occurrence is F; m is 2; and X is O.

In another embodiment, the present invention provides compositions comprising compounds of Formula (IA) or (IB), or a pharmaceutically acceptable salt or hydrate thereof, wherein R₁ is methyl; R₂ is methyl, ethyl, or isopropyl; R₃ is H; A is

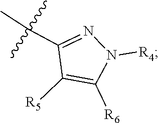

R₄ is methyl; Rₛ is methyl, ethyl, methoxy, Cl, difluoromethoxy, cyano, or cyclopropyl; R₆ is H; B is

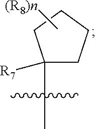

R₇ is methyl; and n is 0.

In another embodiment, the present invention provides compositions comprising compounds of Formula (IIA)

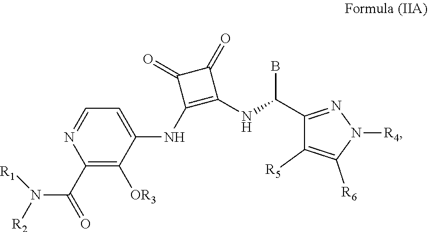

or a pharmaceutically acceptable salt or hydrate thereof, wherein R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl; R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl; R₄ is (C₁-C₄)alkyl; R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, or halo(C₁-C₄)alkyl; B is

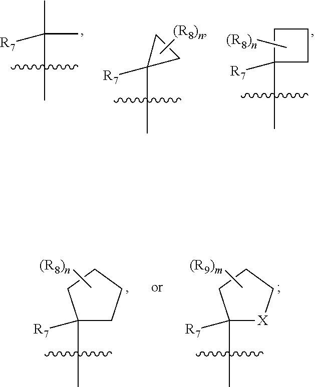

R₇ is (C₁-C₃)alkyl or C₁-C₃)alkyl-d₁₋₇; R₈ is deuterium or two R₈s, together with the same carbon atom to which the two R₈s are attached, form a (C₃-C₅)cycloalkyl group; n is 0 or 2; R₉ at each occurrence is F; m is 2; and X is O.

In another embodiment, the present invention provides compositions comprising compounds of Formula (IIA), or a pharmaceutically acceptable salt or hydrate thereof, wherein R₁ is methyl; R₂ is methyl, ethyl, or isopropyl; R₃ is H; R₄ is methyl; Rₛ is methyl, ethyl, methoxy, Cl, difluoromethoxy, cyano, or cyclopropyl; R₆ is H; B is

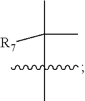

and R₇ is methyl.

In another embodiment, the present invention provides compositions comprising compounds of Formula (IIA), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, ethyl, methoxy, Cl, difluoromethoxy, cyano, or cyclopropyl; R₆ is H; B is

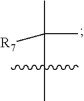

and R₇ is methyl.

In another embodiment, the present invention provides compositions comprising compounds of Formula (IIA), or a pharmaceutically acceptable salt or hydrate thereof, wherein R₁ is methyl; R₂ is methyl, ethyl, or isopropyl; R₃ is H; R₄ is methyl; R₅ is methyl, ethyl, methoxy, Cl, difluoromethoxy, cyano, or cyclopropyl; R₆ is H; B is

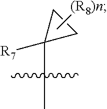

and R₇ is methyl; and n is 0.

In another embodiment, the present invention provides compositions comprising compounds of Formula (IIA), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, methyl-d₃, ethyl, methoxy, Cl, difluoromethoxy, cyano, or cyclopropyl; R₆ is H; B is

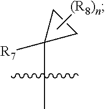

R₇ is methyl; and n is 0.

In another embodiment, the present invention provides compositions comprising compounds of Formula (IIA), or a pharmaceutically acceptable salt or hydrate thereof, wherein R₁ is methyl; R₂ is methyl, ethyl, or isopropyl; R₃ is H; R₄ is methyl; R₅ is methyl, methyl-d₃, ethyl, methoxy, Cl, difluoromethoxy, cyano, or cyclopropyl; R₆ is H; B is

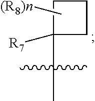

R₇ is methyl; two R₈s, together with the same carbon atom to which they are attached, form cyclopropyl; and n is 0 or 2.

In another embodiment, the present invention provides compositions comprising compounds of Formula (IIA), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, ethyl, methoxy, Cl, difluoromethoxy, cyano, or cyclopropyl; R₆ is H; B is

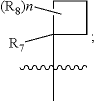

R₇ is methyl; two R₈s, together with the same carbon atom to which they are attached, form cyclopropyl; and n is 0 or 2.

In another embodiment, the present invention provides compositions comprising compounds of Formula (IIA), or a pharmaceutically acceptable salt or hydrate thereof, wherein R₁ is methyl; R₂ is methyl, ethyl, or isopropyl; R₃ is H; R₄ is methyl; R₅ is methyl, methyl-d₃, ethyl, methoxy, Cl, difluoromethoxy, cyano, or cyclopropyl; R₆ is H; B is

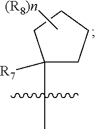

R₇ is methyl; R₃ is deuterium; and n is 0 or 2.

In another embodiment, the present invention provides compositions comprising compounds of Formula (IIA), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, ethyl, methoxy, Cl, difluoromethoxy, cyano, or cyclopropyl; R₆ is H; B is

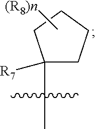

R₇ is methyl; and n is 0.

In another embodiment, the present invention provides compositions comprising compounds of Formula (IIA), or a pharmaceutically acceptable salt or hydrate thereof, wherein R₁ is methyl; R₂ is methyl, ethyl, or isopropyl; R₃ is H; R₄ is methyl; R₅ is methyl, ethyl, methoxy, Cl, difluoromethoxy, cyano, or cyclopropyl; R₆ is H; B is

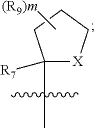

R₇ is methyl; Rₛ is F; m is 2; and X is O.

In another embodiment, the present invention provides compositions comprising compounds of Formula (IIA), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, ethyl, methoxy, Cl, difluoromethoxy, cyano, or cyclopropyl; R₆ is H; B is

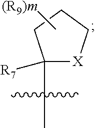

R₇ is methyl; R₉ is F; m is 2; and X is O.

In another embodiment the compound of Formula (IIA) is (R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, or a pharmaceutically acceptable salt or hydrate thereof.

In another embodiment the compound of Formula (IIA) is (R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 18.7±0.2, 19.1±0.2, and 20.2±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 17.6±0.2, 18.4±0.2, 18.7±0.2, 19.1±0.2, and 20.2±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 11.4±0.2, 15.5±0.2, 17.6±0.2, 18.4±0.2, 18.7±0.2, 19.1±0.2, 20.2±0.2, and 24.3±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 11.4±0.2, 12.4±0.2, 15.5±0.2, 17.6±0.2, 18.4±0.2, 18.7±0.2, 19.1±0.2, 20.2±0.2, 24.3±0.2, 26.8±0.2, and 30.5±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising 3 to 10 diffraction peaks selected from the group consisting of 11.4±0.2, 12.4±0.2, 15.5±0.2, 17.6±0.2, 18.4±0.2, 18.7±0.2, 19.1±0.2, 20.2±0.2, 24.3±0.2, 26.8±0.2, and 30.5±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, 0.5 calcium salt, monohydrate.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, 0.5 calcium salt, monohydrate having an X-ray powder diffraction pattern comprising diffraction peaks 7.8±0.2, 10.3±0.2, and 10.7±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, 0.5 calcium salt, monohydrate having an X-ray powder diffraction pattern comprising diffraction peaks 7.8±0.2, 10.3±0.2, 10.7±0.2, 15.5±0.2, and 18.4±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, 0.5 calcium salt, monohydrate having an X-ray powder diffraction pattern comprising diffraction peaks 7.7±0.2, 7.8±0.2, 10.3±0.2, 10.7±0.2, 15.5±0.2, 17.0±0.2, 18.4±0.2, 20.8±0.2, and 21.0±0.2 degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, 0.5 calcium salt, monohydrate having an X-ray powder diffraction pattern comprising diffraction peaks 7.7±0.2, 7.8±0.2, 10.3±0.2, 10.7±0.2, 15.5±0.2, 17.0±0.2, 18.4±0.2, 20.5±0.2, 20.8±0.2, 21.0±0.2, 24.0±0.2, and 25.6±0.2, degrees two theta.

In another embodiment, the present invention provides crystalline (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, 0.5 calcium salt, monohydrate having an X-ray powder diffraction pattern comprising diffraction peaks 7.7±0.2, 7.8±0.2, 10.3±0.2, 10.7±0.2, 11.9±0.2, 13.6±0.2, 15.5±0.2, 16.6±0.2, 17.0±0.2, 18.4±0.2, 20.5±0.2, 20.8±0.2, 21.0±0.2, 22.3±0.2, 24.0±0.2, 24.9±0.2, 25.6±0.2, 26.1±0.2, 31.3±0.2, and 31.4±0.2, degrees two theta.

In another embodiment, the present invention provides crystalline (R)-4-((2-(((4-chloro-1-methyl-1H-pyrazol-3-yl)(1-methylcyclopentyl)methyl)amino)-3,4-dioxocyclobut-1-en-1-yl)amino)-3-hydroxy-N,N-dimethylpicolinamide.

In another embodiment, the present invention provides crystalline (R)-4-((2-(((4-chloro-1-methyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate.

In another embodiment, the present invention provides crystalline (R)-4-((2-(((4-chloro-1-methyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 18.5±0.2, 18.8±0.2, and 19.2±0.2 degrees two theta.

In another embodiment, the present invention provides crystalline (R)-4-((2-(((4-chloro-1-methyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 8.4±0.2, 11.5±0.2, 12.5±0.2, 18.5±0.2, 18.8±0.2, and 19.2±0.2 degrees two theta.

In another embodiment, the present invention provides crystalline (R)-4-((2-(((4-chloro-1-methyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising diffraction peaks 8.4±0.2, 9.3±0.2, 11.5±0.2, 12.5±0.2, 18.5±0.2, 18.8±0.2, 19.2±0.2, and 20.4±0.2 degrees two theta.

In another embodiment, the present invention provides crystalline (R)-4-((2-(((4-chloro-1-methyl-1H-pyrazol-3-yl)(1-methylcyclopentyl)methyl)amino)-3,4-dioxocyclobut-1-en-1-yl)amino)-3-hydroxy-N,N-dimethylpicolinamide monohydrate having an X-ray powder diffraction pattern comprising 3 to 10 diffraction peaks selected from the group consisting of 8.4±0.2, 9.3±0.2, 11.5±0.2, 12.5±0.2, 18.5±0.2, 18.8±0.2, 19.2±0.2, 20.4±0.2, 24.5±0.2, 25.1±0.2, and 26.7±0.2 degrees two theta.

In another embodiment, the present invention provides compositions comprising compounds of Formula (IIB)

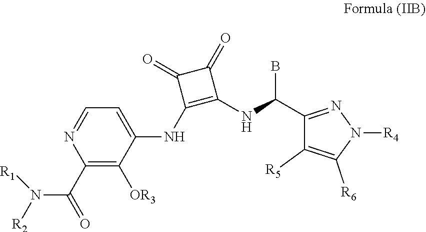

or a pharmaceutically acceptable salt or hydrate thereof, wherein R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl; R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl; R₄ is (C₁-C₄)alkyl; R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, or halo(C₁-C₄)alkyl; B is

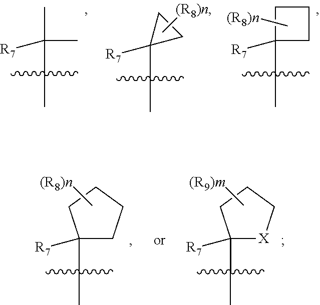

R₇ is (C₁-C₃)alkyl or C₁-C₃)alkyl-d₁₋₇; R₃ is deuterium or two R₉s, together with the same carbon atom to which the two R₈s are attached, form a (C₃-C₅)cycloalkyl group; n is 0 or 2; R₅ at each occurrence is F; m is 2; and X is 0.

In another embodiment, the present invention provides compositions comprising compounds of Formula (IIB), or a pharmaceutically acceptable salt or hydrate thereof, wherein R₁ is methyl; R₂ is methyl, ethyl, or isopropyl; R₃ is H; R₄ is methyl; R₅ is methyl, ethyl, methoxy, Cl, difluoromethoxy, cyano, or cyclopropyl; Rₑ is H; B is

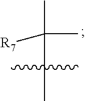

R₇ is methyl.

In another embodiment, the present invention provides compositions comprising compounds of Formula (IIB), or a pharmaceutically acceptable salt or hydrate thereof, wherein R₁ and R₂ taken together with the nitrogen atom to which they are attached form morpholine or 4-methylpiperazine; R₃ is H; R₄ is methyl; Rₛ is methyl, ethyl, methoxy, Cl, difluoromethoxy, cyano, or cyclopropyl; R₆ is H; B is

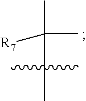

R₇ is methyl.

In another embodiment, the present invention provides compositions comprising compounds of Formula (IIB), or a pharmaceutically acceptable salt or hydrate thereof, wherein R₁ is methyl; R₂ is methyl, ethyl, or isopropyl; R₃ is H; R₄ is methyl; R₅ is methyl, ethyl, methoxy, Cl, difluoromethoxy, cyano, or cyclopropyl; R₆ is H; B is

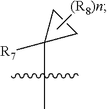

R₇ is methyl; and n is 0.

In another embodiment, the present invention provides compositions comprising compounds of Formula (IIB), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, methyl-d₃, ethyl, methoxy, Cl, difluoromethoxy, cyano, or cyclopropyl; R₆ is H; B is

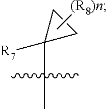

R₇ is methyl; and n is 0.

In another embodiment, the present invention provides compositions comprising compounds of Formula (IIB), or a pharmaceutically acceptable salt or hydrate thereof, wherein R₁ is methyl; R₂ is methyl, ethyl, or isopropyl; R₃ is H; R₄ is methyl; R₅ is methyl, methyl-d₃, ethyl, methoxy, Cl, difluoromethoxy, cyano, or cyclopropyl; R₆ is H; B is

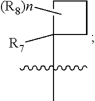

R₇ is methyl; two R₈s, together with the same carbon atom to which they are attached, form cyclopropyl; and n is 0 or 2.

In another embodiment, the present invention provides compositions comprising compounds of Formula (IIB), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, ethyl, methoxy, Cl, difluoromethoxy, cyano, or cyclopropyl; R₆ is H; B is

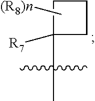

R₇ is methyl; two R₈s, together with the same carbon atom to which they are attached, form cyclopropyl; and n is 0 or 2.

In another embodiment, the present invention provides compositions comprising compounds of Formula (IIB), or a pharmaceutically acceptable salt or hydrate thereof, wherein R₁ is methyl; R₂ is methyl, ethyl, or isopropyl; R₃ is H; R₄ is methyl; R₅ is methyl, methyl-d₃, ethyl, methoxy, Cl, difluoromethoxy, cyano, or cyclopropyl; R₆ is H; B is

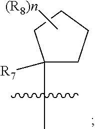

R₇ is methyl; R₃ is deuterium; and n is 0 or 2.

In another embodiment, the present invention provides compositions comprising compounds of Formula (IIB), or a pharmaceutically acceptable salt or hydrate thereof, wherein R₁ and R₂ taken together with the nitrogen atom to which they are attached form morpholine or 4-methylpiperazine; R₃ is H; R₄ is methyl; Rₛ is methyl, ethyl, methoxy, Cl, difluoromethoxy, cyano, or cyclopropyl; R₆ is H; B is

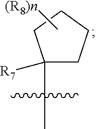

R₇ is methyl; and n is 0.

In another embodiment, the present invention provides compositions comprising compounds of Formula (IIB), or a pharmaceutically acceptable salt or hydrate thereof, wherein R₁ is methyl; R₂ is methyl, ethyl, or isopropyl; R₃ is H; R₄ is methyl; Rₛ is methyl, ethyl, methoxy, Cl, difluoromethoxy, cyano, or cyclopropyl; R₆ is H; B is

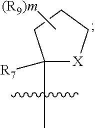

R₇ is methyl; R₉ is F; m is 2; and X is O.

In another embodiment, the present invention provides compositions comprising compounds of Formula (IIB), or a pharmaceutically acceptable salt or hydrate thereof, wherein R₁ and R₂ taken together with the nitrogen atom to which they are attached form morpholine or 4-methylpiperazine; R₃ is H; R₄ is methyl; R₅ is methyl, ethyl, methoxy, Cl, difluoromethoxy, cyano, or cyclopropyl; R₆ is H; B is

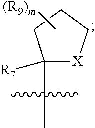

R₇ is methyl; Rₛ is F; m is 2; and X is O.

In another embodiment the compound of Formula (IIB) is (S)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, or a pharmaceutically acceptable salt or hydrate thereof.

In another embodiment the compound of Formula (IIB) is (S)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide.

In another embodiment, the present invention provides compositions comprising compounds of Formula (IIIA)

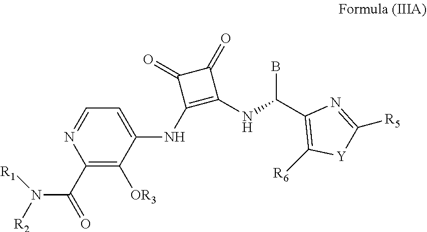

or a pharmaceutically acceptable salt or hydrate thereof, wherein Y is O or S; R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl; R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl; R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, or halo(C₁-C₄)alkyl; B is

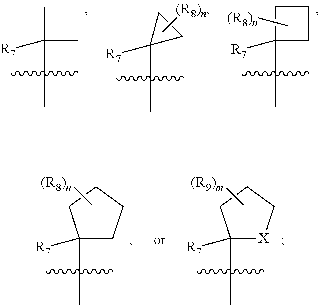

R₇ is (C₁-C₃)alkyl or (C₁-C₃)alkyl-d₁₋₇; R₈ is deuterium or two R₈s, together with the same carbon atom to which the two R₈s are attached, form a (C₃-C₅)cycloalkyl group; n is 0 or 2; R₉ at each occurrence is F; m is 2; and X is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

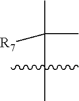

R₇ is methyl.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl, ethyl, methoxy, Cl, difluoromethoxy, cyano, cyclopropyl, or hydroxymethyl; R₆ is methyl or methoxy; B is

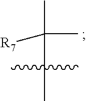

R₇ is methyl.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

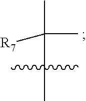

R₇ is methyl.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

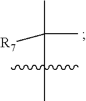

R₇ is methyl.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; Rₛ is methyl, ethyl, methoxy, Cl, difluoromethoxy, cyano, cyclopropyl, or hydroxymethyl; R₆ is methyl or methoxy; B is

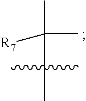

R₇ is methyl.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

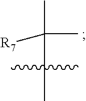

R₇ is methyl.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

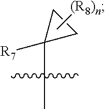

R₇ is methyl; and n is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; Rₛ is methyl, ethyl, methoxy, Cl, difluoromethoxy, cyano, cyclopropyl, or hydroxymethyl; R₆ is methyl or methoxy; B is

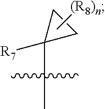

R₇ is methyl; and n is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

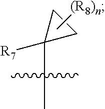

R₇ is methyl; and n is 0.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

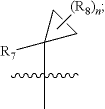

R₇ is methyl; and n is 0.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl, ethyl, methoxy, Cl, difluoromethoxy, cyano, cyclopropyl, or hydroxymethyl; R₆ is methyl or methoxy; B is

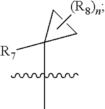

R₇ is methyl; and n is 0.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

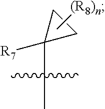

R₇ is methyl; and n is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

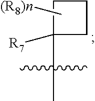

R₇ is methyl; two R₈s, together with the same carbon atom to which they are attached, form cyclopropyl; and n is 0 or 2.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl, ethyl, methoxy, Cl, difluoromethoxy, cyano, cyclopropyl, or hydroxymethyl; R₆ is methyl or methoxy; B is

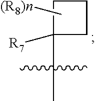

R₇ is methyl; two R₈s, together with the same carbon atom to which they are attached, form cyclopropyl; and n is 0 or 2.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

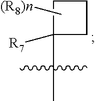

R₇ is methyl; two R₈s, together with the same carbon atom to which they are attached, form cyclopropyl; and n is 0 or 2.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

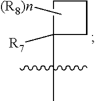

R₇ is methyl; two R₈s, together with the same carbon atom to which they are attached, form cyclopropyl; and n is 0 or 2.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; Rₛ is methyl, ethyl, methoxy, Cl, difluoromethoxy, cyano, cyclopropyl, or hydroxymethyl; R₆ is methyl or methoxy; B is

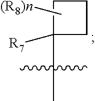

R₇ is methyl; two R₈s, together with the same carbon atom to which they are attached, form cyclopropyl; and n is 0 or 2.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

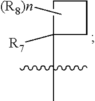

R₇ is methyl; two R₈s, together with the same carbon atom to which they are attached, form cyclopropyl; and n is 0 or 2.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

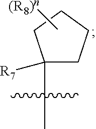

R₇ is methyl; and n is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl, ethyl, methoxy, Cl, difluoromethoxy, cyano, cyclopropyl, or hydroxymethyl; R₆ is methyl or methoxy; B is

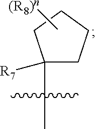

R₇ is methyl; and n is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

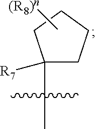

R₇ is methyl; and n is 0.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

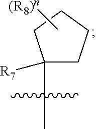

R₇ is methyl; and n is 0.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl, ethyl, methoxy, Cl, difluoromethoxy, cyano, cyclopropyl, or hydroxymethyl; R₆ is methyl or methoxy; B is

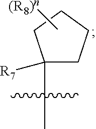

R₇ is methyl; and n is 0.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

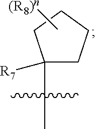

R₇ is methyl; and n is 0.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

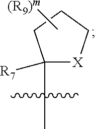

R₇ is methyl; R₉ is F; m is 2; and X is O.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; Rₛ is methyl, ethyl, methoxy, Cl, difluoromethoxy, cyano, cyclopropyl, or hydroxymethyl; R₆ is methyl or methoxy; B is

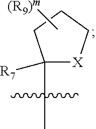

R₇ is methyl; R₉ is F; m is 2; and X is O.

In another embodiment, the present invention provides compositions comprising compounds of Formula (IIIA), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

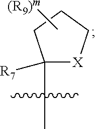

R₇ is methyl; R₉ is F; m is 2; and X is O.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

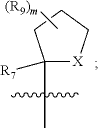

R₇ is methyl; R₉ is F; m is 2; and X is O.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; Rₛ is methyl, ethyl, methoxy, Cl, difluoromethoxy, cyano, cyclopropyl, or hydroxymethyl; R₆ is methyl or methoxy; B is

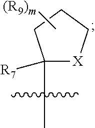

R₇ is methyl; R₉ is F; m is 2; and X is O.

In another embodiment, the present invention provides compositions comprising compounds of Formula (IIIA), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

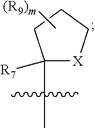

R₇ is methyl; R₉ is F; m is 2; and X is O.

In another embodiment, the present invention provides compositions comprising compounds of Formula (IIIB)

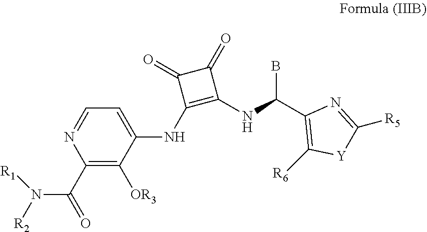

or a pharmaceutically acceptable salt or hydrate thereof, wherein Y is O or S; R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl; R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl; R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, or halo(C₁-C₄)alkyl; B is

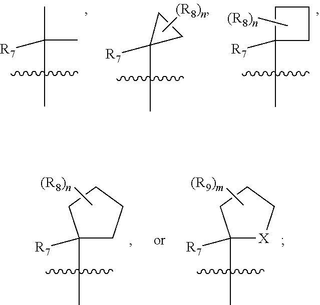

R₇ is (C₁-C₃)alkyl or (C₁-C₃)alkyl-d₁₋₇; R₈ is deuterium or two R₈s, together with the same carbon atom to which the two R₈s are attached, form a (C₃-C₅)cycloalkyl group; n is 0 or 2; R₉ at each occurrence is F; m is 2; and X is O.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

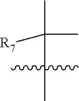

R₇ is methyl.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; Rₛ is methyl, ethyl, methoxy, Cl, difluoromethoxy, cyano, cyclopropyl, or hydroxymethyl; R₆ is methyl or methoxy; B is

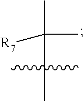

R₇ is methyl.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

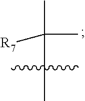

R₇ is methyl.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

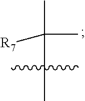

R₇ is methyl.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl, ethyl, methoxy, Cl, difluoromethoxy, cyano, cyclopropyl, or hydroxymethyl; R₆ is methyl or methoxy; B is

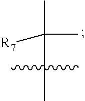

R₇ is methyl.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or

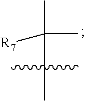

R₇ is methyl.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

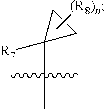

R₇ is methyl; and n is 0.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl, ethyl, methoxy, Cl, difluoromethoxy, cyano, cyclopropyl, or hydroxymethyl; R₆ is methyl or methoxy; B is

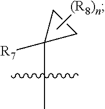

R₇ is methyl; and n is 0.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

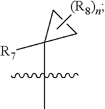

R₇ is methyl; and n is 0.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

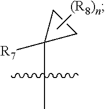

R₇ is methyl; and n is 0.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl, ethyl, methoxy, Cl, difluoromethoxy, cyano, cyclopropyl, or hydroxymethyl; R₆ is methyl or methoxy; B is

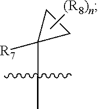

R₇ is methyl; and n is 0.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

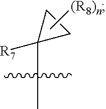

R₇ is methyl; and n is 0.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

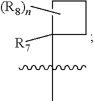

R₇ is methyl; two R₈s, together with the same carbon atom to which they are attached, form cyclopropyl; and n is 0 or 2.

In another embodiment, the present invention provides compositions comprising compounds of Formula (IIIB), or a pharmaceutically acceptable salt or hydrate thereof, wherein Y is O or S; R₂ is methyl, ethyl, or isopropyl; R₃ is H; R₅ is methyl, ethyl, methoxy, Cl, difluoromethoxy, cyano, cyclopropyl, or hydroxymethyl; B is

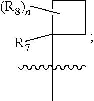

R₇ is methyl; two R₈s, together with the same carbon atom to which they are attached, form cyclopropyl; and n is 0 or 2.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

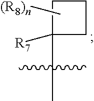

R₇ is methyl; two R₈s, together with the same carbon atom to which they are attached, form cyclopropyl; and n is 0 or 2.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

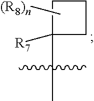

R₇ is methyl; two R₈s, together with the same carbon atom to which they are attached, form cyclopropyl; and n is 0 or 2.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; Rₛ is methyl, ethyl, methoxy, Cl, difluoromethoxy, cyano, cyclopropyl, or hydroxymethyl; R₆ is methyl or methoxy; B is

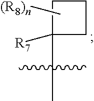

R₇ is methyl; two R₈s, together with the same carbon atom to which they are attached, form cyclopropyl; and n is 0 or 2.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

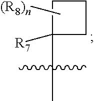

R₇ is methyl; two R₈s, together with the same carbon atom to which they are attached, form cyclopropyl; and n is 0 or 2.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

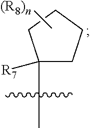

R₇ is methyl; and n is 0.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl, ethyl, methoxy, Cl, difluoromethoxy, cyano, cyclopropyl, or hydroxymethyl; R₆ is methyl or methoxy; B is

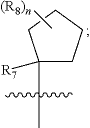

R₇ is methyl; and n is 0.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

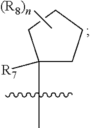

R₇ is methyl; and n is 0.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

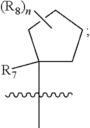

R₇ is methyl; and n is 0.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; Rₛ is methyl, ethyl, methoxy, Cl, difluoromethoxy, cyano, cyclopropyl, or hydroxymethyl; R₆ is methyl or methoxy; B is

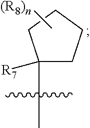

R₇ is methyl; and n is 0.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

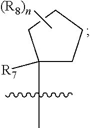

R₇ is methyl; and n is 0.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ and R₆ are independently methyl, ethyl, methoxy, Cl, difluoromethoxy, cyano, cyclopropyl, or hydroxymethyl; B is

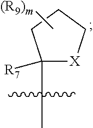

R₇ is methyl; R₉ is F; m is 2; and X is O.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; Rₛ is methyl, ethyl, methoxy, Cl, difluoromethoxy, cyano, cyclopropyl, or hydroxymethyl; R₆ is methyl or methoxy; B is

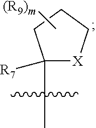

R₇ is methyl; R₉ is F; m is 2; and X is O.

In another embodiment, the present invention provides compositions comprising compounds of Formula (IIIB), or a pharmaceutically acceptable salt or hydrate thereof, wherein Y is O or S; R₁ is methyl; R₂ is methyl, ethyl, or isopropyl; R₃ is H; R₅ is methyl or hydroxymethyl; R₆ is methyl or methoxy; B is

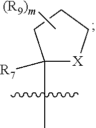

R₇ is methyl; R₉ is F; m is 2; and X is O.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ and R₆ are independently methyl, ethyl, methoxy, Cl, difluoromethoxy, cyano, cyclopropyl, or hydroxymethyl; B is

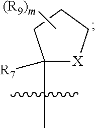

R₇ is methyl; R₉ is F; m is 2; and X is O.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl, ethyl, methoxy, Cl, difluoromethoxy, cyano, cyclopropyl, or hydroxymethyl; R₆ is methyl or methoxy; B is

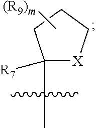

R₇ is methyl; R₉ is F; m is 2; and X is O.

In another embodiment, the present invention provides compositions comprising compounds of Formula (IIIB), or a pharmaceutically acceptable salt or hydrate thereof, wherein Y is O or S; R₁ and R₂ taken together with the nitrogen atom to which they are attached form morpholine or 4-methylpiperazine; R₃ is H; R₅ is methyl or hydroxymethyl; R₆ is methyl or methoxy; B is

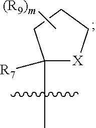

R₇ is methyl; R₉ is F; m is 2; and X is O.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula (IA), (IB), (IIA), (IIB), (IIIA), or (IIIB) may also include other salts of such compounds which are not necessarily pharmaceutically acceptable salts, which may be useful as intermediates for one or more of the following: 1) preparing compounds of Formula (IA), (IB), (IIA), (IIB), (IIIA), or (IIIB); 2) purifying compounds of Formula (IA), (IB), (IIA), (IIB), (IIIA), or (IIIB); 3) separating enantiomers of compounds of Formula (IA), (IB), (IIA), (IIB), (IIIA), or (IIIB); or 4) separating diastereomers of compounds of Formula (IA), (IB), (IIA), (IIB), (IIIA), or (IIIB).

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

Representative examples include, but are not limited to, the compounds shown below wherein R⁺ is lithium, sodium, potassium, ammonium, tetramethylammonium, tetraethylammonium, methylammonium, dimethylammonium, trimethylammonium, triethylammonium, diethylammonium, ethylammonium and the like, and wherein R²⁺ is calcium, magnesium, aluminum, and the like.

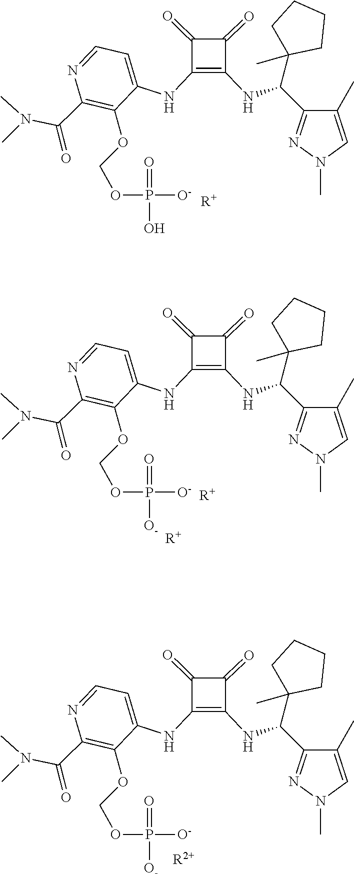

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt or hydrate thereof, and one or more pharmaceutically acceptable solvent molecules, for example, ethanol. The term ‘hydrate’ is employed when said solvent is water.

In addition, the compounds of Formula (IA), (IB), (IIA), (IIB), (IIIA), or (IIIB), or a pharmaceutically acceptable salt or hydrate thereof, may also include other solvates of such compounds which are not necessarily pharmaceutically acceptable solvates, which may be useful as intermediates for one or more of the following: 1) preparing compounds of Formula (IA), (IB), (IIA), (IIB), (IIIA), or (IIIB), or a pharmaceutically acceptable salt or hydrate thereof, 2) purifying compounds of Formula (IA), (IB), (IIA), (IIB), (IIIA), or (IIIB), or a pharmaceutically acceptable salt or hydrate thereof, 3) separating enantiomers of compounds of Formula (IA), (IB), (IIA), (IIB), (IIIA), or (IIIB), or a pharmaceutically acceptable salt or hydrate thereof, or 4) separating diastereomers of compounds of Formula (IA), (IB), (IIA), (IIB), (IIIA), or (IIIB), or a pharmaceutically acceptable salt or hydrate thereof.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid.

Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

In particular, the stereochemistry at the point of attachment of the variables “A” and “B”

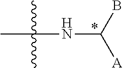

of Formulae (IA), (IB), (IIA), (IIB), (IIIA), and (IIIB) may independently be either (R) or (S). The enantiomers of the present invention indicated by (R), (S), or * are substantially free of the other enantiomer. “Substantially free” means that the enantiomeric excess is greater than about 90%, preferably greater than about 95%, and more preferably greater than about 99%. Within the context of enantiomeric excess, the term “about” means±1.0%. The symbol * designates a chiral carbon atom as either (R) or (S) stereochemistry depending on the configuration of substituents around the chiral carbon atom.

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

In particular, the bis(amino)cyclobut-3-ene-1,2-dione moiety contained within the compounds of the present invention may tautomerize as shown below and are included within the scope of the present invention.

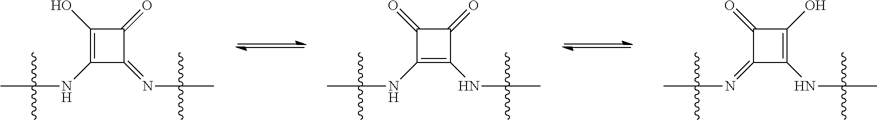 [SMILES: */N=c1/c(N*)c(O)c1=O ; *Nc1c(N*)c(=O)c1=O ; */N=c1/c(N*)c(O)c1=O]

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S Certain isotopically-labelled compounds of the invention, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In particular, the present invention includes deuterated compounds of Formula (IA), (IB), (IIA), (IIB), (IIIA), and (IIIB). Any of the hydrogens contained on the compounds of the present invention may be exchanged for deuterium including the pyridine hydrogens as shown below.

Representative examples of deuterated compounds of the present invention include, but are not limited to, Examples 39 and 40. In some embodiments, one or more hydrogen atoms on certain metabolic sites on the compounds of the invention are deuterated.

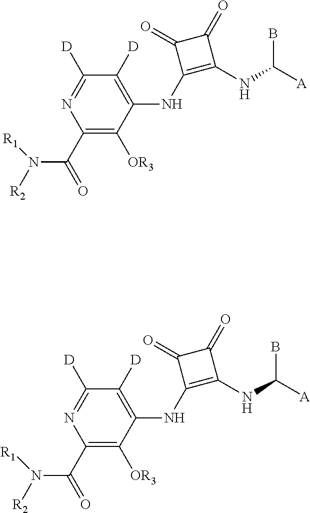

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio, et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH→—COH):
(ii) where the compound of the invention contains an alkoxy group, a hydroxy derivative thereof (—OR→—OH);
(iii) where the compound of the invention contains a tertiary amino group, a secondary amino derivative thereof (—NRR′→—NHR or —NHR′);
(iv) where the compound of the invention contains a secondary amino group, a primary derivative thereof (—NHR→—NH₂);
(v) where the compound of the invention contains a phenyl moiety, a phenol derivative thereof (-Ph→-PhOH);
(vi) where the compound of the invention contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide.

Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions. For pharmaceutical composition purposes, the compound per se or pharmaceutically acceptable salt or hydrate thereof will simply be referred to as the compounds of the invention.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a pharmaceutical composition which enhances absorption or penetration of the active ingredient through the skin or other affected areas. When the pharmaceutical compositions of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the pharmaceutical compositions of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the pharmaceutical compositions of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures.

The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Pharmaceutical compositions of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil.

The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Co-Process and Co-Precipitation of Compounds of the Disclosure

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability.

Co-processing is a process in which commonly used excipients can be combined with an API in a more deliberate and controlled manner to design a pharmaceutical composite material. Co-processing techniques include co-spray drying, melt granulation, dry coating, and simultaneous or subsequent precipitation of API and/or excipients in solvent media such as the adsorption of the drug in a porous carrier, morphology modification by habit modifier additives, heteroepitaxial crystallization, and controlled agglomeration.

Co-precipitation (CPT) is a method of simultaneously precipitating at least one compound from a solution. In one embodiment, a composition of the disclosure is prepared using co-precipitation with a solution comprising a compound disclosed herein. In some embodiments, the compound is isolated and stabilized in the amorphous state, for example, in an amorphous solid dispersion (ASD). In some embodiments, spray dry dispersion (SDD), melt extrudates, co-precipitates, amorphous drug nanoparticles, and nano-adsorbates are used to prepare an amorphous solid dispersion.

In one embodiment, amorphous solid dispersions comprise a compound of the invention and a polymer. In some embodiments, the polymer comprises a polyvinyl lactam polymer, a cellulose derivative polymer, or a polymethacrylate polymer, or a mixture thereof. In one embodiment, a composition of the disclosure comprises a compound of Formula (IA), (IB), (IIA), (IIB), (IIIA), or (IIIB), or a pharmaceutically acceptable salt or hydrate thereof; and a polyvinyl lactam polymer. In some embodiments, the polyvinyl lactam polymer is selected from the group consisting of: polyvinylpyrrolidone (PVP), polyvinylpyrrolidone-vinyl acetate copolymer (PVP/VA), or a polyvinyl caprolactam-polyvinyl acetate-polyethylene glycol graft polymer. In one embodiment, the polyvinyl lactam polymer is PVP. In one embodiment, a composition of the disclosure comprises a compound of Formula (IA), (IB), (IIA), (III), (IIIA), or (IIIB), or a pharmaceutically acceptable salt or hydrate thereof; and a methylcellulose derivative polymer. In some embodiments, the methylcellulose derivative polymer is hydroxypropyl cellulose (HPC), hydroxypropyl methylcellulose (HPMC), hydroxy ethyl cellulose, Hypromellose acetate succinate (HPMCAS), hydroxypropyl methylcellulose phthalate (HPMCP), and cellulose acetate phthalate (CAP), or a mixture thereof. In one embodiment, the cellulose derivative polymer is HPMCAS. In one embodiment, a composition of the disclosure comprises a compound of Formula (IA), (IB), (IIA), (IIB), (IIIA), or (IIIB), or a pharmaceutically acceptable salt or hydrate thereof; and a methacrylate polymer.

In some embodiments, a composition disclosed herein comprises from about 5% to about 45% of a compound of Formula (IA), (IB), (IIA), (IIB), (IIIA), or (IIIB), or a pharmaceutically acceptable salt or hydrate thereof; and from about 55% to about 95% of a polymer. In some embodiments, a composition disclosed herein comprises from about 15% to about 30% of a compound of Formula (IA), (IB), (IIA), (IIB), (IIIA), or (IIIB), or a pharmaceutically acceptable salt or hydrate thereof; and from about 70% to about 85% of a polymer. In some embodiments, a composition disclosed herein comprises about 5%, about 10%, about 15%, about 20%, about 25%, about 30%, about 35%, about 40%, or about 45% of a compound of Formula (IA), (IB), (IIA), (IIB), (IIIA), or (IIIB), or a pharmaceutically acceptable salt or hydrate thereof; and about 55%, about 60%, about 65%, about 70%, about 75%, about 80%, about 85%, about 90%, or about 95% of a polymer. In some embodiments, a composition disclosed herein comprises about 20%, about 25%, about 30% of a compound of Formula (IA), (IB), (IIA), (IIB), (IIIA), or (IIIB), or a pharmaceutically acceptable salt or hydrate thereof; and 70%, about 75%, about 80% of a polymer. In one embodiment, a composition disclosed herein comprises about 25% of a compound of Formula (IA), (IB), (IIA), (IIB), (IIIA), or (IIIB), or a pharmaceutically acceptable salt or hydrate thereof; and about 75% of a polymer.

### Administration and Dosing

Typically, a pharmaceutical composition of the invention is administered in an amount effective to treat a condition as described herein. The pharmaceutical compositions of the invention may be administered with the compound per se, or alternatively, as a pharmaceutically acceptable salt. For administration and dosing purposes, the compound per se or pharmaceutically acceptable salt or hydrate thereof will simply be referred to as the compounds of the invention.

The pharmaceutical compositions described herein are administered in a dose effective for the treatment intended. The pharmaceutical compositions of the invention may be administered orally, rectally, vaginally, parenterally, topically, intranasally, or by inhalation.

The pharmaceutical compositions of the invention may be administered orally. Oral administration may involve swallowing, so that the compound enters the gastrointestinal tract, or buccal or sublingual administration may be employed by which the compound enters the bloodstream directly from the mouth.

In another embodiment, the pharmaceutical compositions of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the pharmaceutical compositions of the invention may also be administered topically to the skin or mucosa, that is, dermally or transdermally. In another embodiment, the pharmaceutical compositions of the invention may also be administered intranasally or by inhalation. In another embodiment, the pharmaceutical compositions of the invention may be administered rectally or vaginally. In another embodiment, the pharmaceutical compositions of the invention may also be administered directly to the eye or ear.

The dosage regimen for the pharmaceutical compositions is based on a variety of factors, including the type, age, weight, sex and medical condition of the patient; the severity of the condition; the route of administration; and the activity of the particular pharmaceutical composition employed. Thus, the dosage regimen may vary widely. In one embodiment, the total daily dose of a pharmaceutical composition of the invention is typically from about 0.01 to about 100 mg/kg of the active pharmaceutical ingredient for the treatment of the indicated conditions discussed herein. In another embodiment, total daily dose of the pharmaceutical composition of the invention is from about 0.1 to about 50 mg/kg, and in another embodiment, from about 0.5 to about 30 mg/kg of the API. It is not uncommon that the administration of the pharmaceutical compositions of the invention will be repeated a plurality of times in a day (typically no greater than 4 times). Multiple doses per day typically may be used to increase the total daily dose, if desired.

### Therapeutic Methods and Uses

The compositions of the invention may inhibit CCR6 and may be useful in the treatment of a condition ameliorated by CCR6 inhibition. In some embodiments, the compositions of the disclosure can treat a condition selected from the group consisting of: psoriasis, ulcerative colitis, Crohn's disease, inflammatory bowel disease, psoriatic arthritis, and rheumatoid arthritis. In some embodiments, the compositions of the disclosure can treat a condition selected from the group consisting of: Crohn's disease, ulcerative colitis, celiac sprue, celiac diseases, proctitis, eosinophilic gastroenteritis, autoimmune atrophic gastritis of pernicious anemia, and mastocytosis. In some embodiments, the compositions of the disclosure can be used to treat inflammatory bowel disease, Crohn's disease, or ulcerative colitis. In some embodiments, the compositions of the disclosure can be used to treat inflammatory bowel disease. In some embodiments, the compositions of the disclosure can be used to treat inflammatory Crohn's disease. In some embodiments, the compositions of the disclosure can be used to treat ulcerative colitis.

In some embodiments, the compositions of the disclosure can be used to treat a condition selected from the group consisting of: rheumatoid arthritis, juvenile arthritis, Still's disease, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile idiopathic arthritis (JIA), juvenile psoriatic arthritis, juvenile rheumatoid arthritis, systemic onset juvenile rheumatoid arthritis, giant cell arteritis, and secondary osteoarthritis from inflammatory diseases.

In some embodiments, the compositions of the disclosure can treat a condition selected from the group consisting of: lupus, systemic lupus erythematosus, juvenile systemic lupus erythematosus, lupus nephritis, Sjögren's syndrome, scleroderma (systemic sclerosis), Raynaud's phenomenon, juvenile scleroderma, polymyositis, dermatomyositis, polymyositis-dermatomyositis, mixed connective tissue disease, sarcoidosis, fibromyalgia, vasculitis microscopic polyangiitis, vasculitis, eosinophilic granulomatosis with polyangiitis (formerly known as Churg-Strauss syndrome), granulomatosis with polyangiitis (formerly known as Wegener's granulomatosis), polyarteritis nodosa, Henoch-Schönlein purpura, idiopathic thrombocytopenic thrombotic purpura, juvenile vasculitis, polyarteritis nodossa (also known as panarteritis nodosa, periarteritis nodosa, Kussmaul disease, Kussmaul-Maier disease or PAN), serum sickness, Myasthenia gravis, Takayasu's arteritis, Behçet's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, sclerosing cholangitis, membranous glomerulopathy, polymyositis, myositis, atherosclerosis, autoimmune hemolytic anemia, autoimmune orchitis, and Goodpasture's disease.

In some embodiments, the compositions of the disclosure can treat a condition selected from the group consisting of: psoriasis, atopic dermatitis, eczema dermatitis, dermatitis, pruritus, alopecia, autoimmune alopecia, vitiligo, epidermal hyperplasia, juvenile dermatomyositis, and dermatomyositis. In certain other embodiments, the psoriasis is plaque psoriasis, Guttate psoriasis, psoriatic epidermal hyperplasia, inverse psoriasis, pustular psoriasis, and erythrodermic psoriasis.

In some embodiments, the compositions of the disclosure can treat a condition selected from the group consisting of: non-alcoholic steatohepatitis, non-alcoholic fatty liver disease, autoimmune hepatitis, chronic aggressive hepatitis, and primary biliary sclerosis. In some embodiments, the compositions of the disclosure can treat a condition selected from the group consisting of: multiple sclerosis, amyotropic lateral sclerosis, Guillain-Barre disease, autoimmune encephalomyelitis, Alzheimer's disease, major depressive disorder, traumatic brain injury, epilepsy, Parkinson's disease, or bipolar disorder

In some embodiments, the compositions of the disclosure can treat a condition selected from the group consisting of: neurodegenerative or neuroinflammatory disorders, an autoimmune disorder, an immune disorder,

In some embodiments, the compositions of the disclosure can treat a condition selected from the group consisting of: Graves' disease, noninfectious uveitis, dry eye syndrome, sympathetic ophthalmia, Cogan's syndrome, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, ocular neovascularization, or proliferative diabetic retinopathy

In some embodiments, the compositions of the disclosure can treat a condition selected from the group consisting of: asthma, allergy, chronic obstructive pulmonary disease, and acute respiratory disease.

In some embodiments, the compositions of the disclosure can treat a condition selected from the group consisting of: alimentary/gastrointestinal tract cancer, colon cancer, liver cancer, skin cancer including mast cell tumor and squamous cell carcinoma, breast and mammary cancer, ovarian cancer, prostate cancer, leukemia, diffuse large B cell lymphoma, cutaneous T-cell lymphoma, non-Hodgkin lymphoma, kidney cancer, lung cancer, muscle cancer, bone cancer, bladder cancer, brain cancer, melanoma including oral and metastatic melanoma, Kaposi's sarcoma, multiple myeloma, myeloproliferative disorders, glioblastoma, oligodendroglioma, pancreatic cancer, brain tumors, or gliomas including astrocytoma. In certain other embodiments, the leukemia is acute myeloid leukemia, T cell acute lymphoblastic leukemia, and adult T cell leukemia

In some embodiments, the compositions of the disclosure can treat a condition selected from the group consisting of: Type I diabetes mellitus, Type II diabetes mellitus, and Juvenile onset diabetes.

In one embodiment, disclosed herein is a method of treating a condition, the method comprising administering to a subject in need thereof a composition comprising: a) a therapeutically effective amount of a compound of Formula (IA), (IB), (IIA), (III), (IIIA), or (IIIB), or a pharmaceutically acceptable salt or hydrate thereof; and b) a polymer, wherein the compound and the polymer are in an amorphous solid dispersion.

In one embodiment, disclosed herein is a composition for use in treating a condition, wherein the composition comprises: a) a therapeutically effective amount of a compound of Formula (IA), (IB), (IIA), (IIB), (IIIA), or (IIIB), or a pharmaceutically acceptable salt or hydrate thereof; and b) a polymer, wherein the compound and the polymer are in an amorphous solid dispersion.

In one embodiment, disclosed herein is a use of a composition for treating a condition, the use comprising administering to a subject in need thereof the composition, wherein the composition comprises: a) a therapeutically effective amount of a compound of Formula (IA), (IB), (IIA), (IIB), (IIIA), or (IIIB), or a pharmaceutically acceptable salt or hydrate thereof; and b) a polymer, wherein the compound and the polymer are in an amorphous solid dispersion.

In one embodiment, disclosed herein is a use of a composition for the manufacture of a medicament for the treatment of a condition, wherein the composition comprises: a) a therapeutically effective amount of a compound of Formula (IA), (IB), (IIA), (III), (IIIA), or (IIIB), or a pharmaceutically acceptable salt or hydrate thereof; and b) a polymer, wherein the compound and the polymer are in an amorphous solid dispersion.

### Co-Administration

The pharmaceutical compositions of the invention may be used alone, or in combination with one or more other therapeutic agents. The invention provides any of the uses, methods or compositions as defined herein wherein the composition of the invention, or pharmaceutically acceptable salt or hydrate thereof, is used in combination with one or more other therapeutic agent discussed herein.

The administration of two or more compounds or compositions “in combination” means that all of the compounds and/or compositions are administered closely enough in time to affect treatment of the subject. The two or more compounds or compositions may be administered simultaneously or sequentially, via the same or different routes of administration, on same or different administration schedules and with or without specific time limits depending on the treatment regimen. Additionally, simultaneous administration may be carried out by mixing the compounds or compositions prior to administration or by administering the compounds or compositions at the same point in time but as separate dosage forms at the same or different site of administration. Examples of “in combination” include, but are not limited to, “concurrent administration,” “co-administration,” “simultaneous administration,” “sequential administration” and “administered simultaneously”.

A composition of the invention and the one or more other therapeutic agents may be administered as a fixed or non-fixed combination of the active ingredients. The term “fixed combination” means a composition of the invention, comprising a compound disclosed herein or a pharmaceutically acceptable salt or hydrate thereof, and the one or more therapeutic agents, are both administered to a subject simultaneously in a single composition or dosage. The term “non-fixed combination” means that a pharmaceutical composition of the invention, comprising a composition disclosed herein or a pharmaceutically acceptable salt or hydrate thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In some embodiments, a composition of the disclosure can be administered in combination with an agent selected from the group consisting of: a PDE4 isoenzyme inhibitor including, but not limited to, ampremilast, roflumilast, rolipram, piclamilast, crisaborole, PF-07038124, PF-07091905, PF-07090414, PF-07062087, PF-07062077, and PF-07057566; a corticosteroid including, but not limited to, fluocinonide, desoximetasone, mometasone, triamcinolone, betamethasone, alclometasone, desonide, hydrocortisone, LEO-134310A and mapracorat; a calcineurin inhibitor including, but not limited to, tacrolimus, pimecrolimus and cyclosporine; a JAK inhibitor including, but not limited to, tofacitinib, JTE-052, baricitinib, upadacitinib, PF-04965842, PF-06651600 and PF-06700841; a TYK inhibitors including, but not limited to, PF-06826647 and BMS-986165; an ITK inhibitor including, but not limited to, JTE-051; a SYK inhibitor including, but not limited to, fostamatinib, cerdulatinib, entospletinib, TAK-659, ASN-002 and GS-9876; a tyrosine kinase inhibitors including, but not limited to, cerdulatinib; an IRAK4 inhibitor including, but not limited to, 1-(((2S,3S,4S)-3-ethyl-4-fluoro-5-oxopyrrolidin-2-yl)methoxy)-7-methoxyisoquinoline-6 carboxamide; an anti-inflammatory agent including, but not limited to, WBI-1001 and MRX-6; retinoic acid derivatives including, but not limited to, alitretinoin; a liver X receptor (LXR) selective agonist including, but not limited to, VTP-38543; a H4 receptor antagonists including, but not limited to, ZPL-389; a NKI receptor antagonists including, but not limited to, Aprepitant and Tradipitant; a CRTH2 receptor antagonists including, but not limited to, Fevipiprant and OC-459; a Chymase inhibitors including, but not limited to, SUN 13834; a GATA-3 inhibitors including, but not limited to, SB-011; and a RORC2 inverse agonist including, but not limited to, PF-06763809, ESR-114, VTP-43742, ARN6039, TAK-828, RTA-1701, BOS-172767, AUR-101 and JTE-451. In one embodiment, a composition of the disclosure can be administered with at least one anti-inflammatory agent to treat an autoimmune disease.

In some embodiments, a composition of the disclosure can be administered in combination with an agent selected from the group consisting of: oral anti-inflammatory agents including, but not limited to, aspirin, ibuprofen, naproxen, sulindac, indomethacin, mefenamate, droxicam, diclofenac, sulfinpyrazone, piroxicam, CELEBREX®, and pharmaceutically acceptable salts or prodrugs thereof; oral retinoic acid derivatives including, but not limited to, alitretinoin; oral liver X receptor (LXR) selective agonists including, but not limited to, VTP-38543; oral H4 receptor antagonists including, but not limited to, ZPL-389; oral NKI receptor antagonists including, but not limited to, Aprepitant and Tradipitant; oral CRTH2 receptor antagonists including, but not limited to, Fevipiprant and OC-459; oral Chymase inhibitors including, but not limited to, SUN 13834; oral GATA-3 inhibitors including, but not limited to, SB-011; oral RORC2 inverse agonists including, but not limited to, ESR-114, VTP-43742, ARN6039, TAK-828, RTA-1701, BOS-172767, AUR-101 and JTE-451; oral JAK inhibitors including, but not limited to, tofacitinib, JTE-052, baricitinib, upadacitinib, PF-04965842, PF-06651600, and PF-06700841; oral TYK inhibitors including, but not limited to, PF-06826647 and BMS-986165; oral ITK inhibitors including, but not limited to, JTE-051; oral SYK inhibitors including, but not limited to, fostamatinib, cerdulatinib, entospletinib, TAK-659, ASN-002 and GS-9876; oral S1P receptor modulators including, but not limited to, ozanimod and etrasimod; and oral IRAK4 inhibitors including, but not limited to, 1-(((2S,3S,4S)-3-ethyl-4-fluoro-5-oxopyrrolidin-2-yl)methoxy)-7-methoxyisoquinoline-6 carboxamide and BAY 1830839.

In some embodiments, a composition of the disclosure can be administered in combination with an agent selected from the group consisting of: TNFα inhibitors including, but not limited to, infliximab, adalimumab, golimumab, and certolizumab pegol; anti-MAdCAM including, but not limited to, SHP647; anti IL-12 P40 including, but not limited to, ustekinumab; anti-IL-23 P19 including, but not limited to, risankizumab, mirikizumab, brazikumab, guselkumab, and tidrakizumab; anti-IL-17 including, but not limited to, secukinumab, brodalumab and ixekizumab; integrin inhibitors including, but not limited to, natalizumab, vedolizumab and etrolizumab.

These agents and composition of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

Combination therapy includes administration of the two or more therapeutic agents concurrently or sequentially. The agents may be administered in any order. Alternatively, the multiple therapeutic agents can be combined into a single composition that can be administered to the patient. For instance, a single pharmaceutical composition could comprise the first composition comprising a compound disclosed herein or pharmaceutically acceptable salt, hydrate, ester or prodrug thereof according to the Formula (IA), (IB), (IIA), (III), (IIIA), or (IIIB), another therapeutic agent or a pharmaceutically acceptable salt, hydrate, ester or prodrug thereof, and at least one pharmaceutically acceptable excipient or carrier.

Kits Another aspect of the invention provides kits comprising the composition of the invention or pharmaceutical compositions comprising the composition of the invention. A kit may include, in addition to the composition of the invention, diagnostic or therapeutic agents. A kit may also include instructions for use in a diagnostic or therapeutic method. In some embodiments, the kit includes a composition thereof and a diagnostic agent. In other embodiments, the kit includes a composition thereof and one or more therapeutic agents described herein.

In yet another embodiment, the invention comprises kits that are suitable for use in performing the methods of treatment described herein. In one embodiment, the kit contains a first dosage form comprising one or more of the compositions of the invention in quantities sufficient to carry out the methods of the invention. In another embodiment, the kit comprises one or more compositions of the invention in quantities sufficient to carry out the methods of the invention and a container for the dosage and a container for the dosage.

### EXAMPLES

### General Methods

The compounds of the disclosure were synthesized using methods described in WO2020058869A1.

The following SFC methods were employed. SFC method A: Chiral Tech OD-H 250 mm×4.6 mm×5 μm, 5 to 60% 0.2% NH₄+(7 N in MeOH) in EtOH, 3.0 mL/min. SFC method B: Chiralpak AD-3 50 mm×3 mm×3 μm, 5 to 40% 0.05% DEA in EtOH, 2.5 mL/min, 40° C. SFC method C: Chiralcel OD 250 mm×4.6 mm×5 μm, 5 to 60% 0.2% NH4⁺ (7 M in MeOH) in EtOH, 3.0 mL/min. SFC method D: Chiralpak AD-3 150 mm×4.6 mm×3 μm, 5 to 40% 0.05% DEA in EtOH, 2.5 mL/min, 40° C. SFC method E: Chiralcel OJ-H 150 mm×4.6 mm×5 μm, 5 to 40% 0.05% DEA in EtOH, 2.5 mL/min, 40° C. SFC method F: Chiralcel OD-3 100 mm×4.6 mm×3 μm, 5 to 40% 0.05% DEA in EtOH, 2.8 mL/min, 40° C. SFC method G: Chiralpak AD-3 150 mm×4.6 mm×3 μm, 5 to 40% 0.05% DEA in IPA, 2.5 mL/min, 40° C. SFC method H: Chiralcel OD-3 150×4.6 mm 1.D. 3 μm, 5 to 40% 0.05% DEA in EtOH, 2.5 mL/min, 40° C.). SFC method I: REGIS (s,s) WHELK-01 250 mm×30 mm×5 μm 40% 0.05% DEA in EtOH, 2.5 mL/min, 35° C. SFC method J: Chiralpak AS-3 150×4.6 mm×3 μm, 5 to 40% 0.05% DEA in EtOH, 2.5 mL/min, 35° C. SFC method K: Chiralpak AS-3 100×4.6 mm×3 μm, 5 to 40% 0.05% DEA in EtOH, 2.8 mL/min, 40° C. SFC method L. Lux Amylose W-1, 250 mm×4.6 mm, 5 μm, 5 to 60% 0.2% NH₃ in EtOH, 3 mL/min. SFC method M. Lux Cellulose, 150 mm×4.6 mm, 3 μm, 5 to 40% MeOH, 2 mL/min. SFC method N: Chiralpak AD-3 150 mm×4.6 mm×3 μm, 5 to 40% 0.1% ethanolamine in EtOH, 2.5 mL/min.

The following HPLC methods have been employed. HPLC method A: Chiralcel OD-RH, 150 mm×4.6 mm×5 μm, 10 to 80% MeCN in 0.069% TFA in H₂O, 0.8 mL/min, 30° C. HPLC method B: Chiralpak AS-RH, 150 mm×4.6 mm×5 μm, 10 to 80% MeCN in 0.069% TFA in H₂O, 0.8 mL/min, 30° C.

### Example 1: (R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide

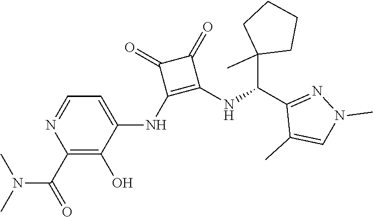

Step 1A. 1-Methylcyclopentane-1-carbonitrile: To a solution of LHMDS (280 mL, 280 mmol, 1 M solution in THF) at −70° C. was added dropwise a solution of cyclopentanecarbonitrile (26.67 g, 280.3 mmol) in THF (20 mL) over 15 min. After stirring for 30 min, iodomethane (59.7 g, 26.2 mL, 420 mmol) was added dropwise, and the reaction was allowed to warm to ambient temperature and stirred for 16 h. The resulting yellow solution was cooled to 0° C. and quenched with saturated aqueous NH₄Cl solution (200 mL) and water (100 mL). The mixture was extracted with MTBE (2.5 L×2), and the combined organic extracts were washed with brine (1 L), dried over Na₂SO₄, and filtered. The filtrate was concentrated in vacuo. The crude product was purified twice using silica gel column chromatography (100% petroleum ether) to afford 60 g (65%) of the title compound as a yellow oil. ¹H NMR (400 MHz, CDC₃) 5 2.20-2.10 (m, 2H), 1.90-1.70 (m, 4H), 1.65-1.55 (m, 2H), 1.41 (s, 3H).

Step 1B. 1-Methylcyclopentane-1-carbaldehyde: To a solution of DIBAL-H (824 mL, 824 mmol, 1 M in toluene) at −65° C. was added dropwise a solution of 1-methylcyclopentane-1-carbonitrile (30 g, 275 mmol) in DCM (30 mL). The mixture was stirred at the same temperature for 30 min. The reaction was quenched with saturated aqueous NH₄Cl solution (1 L) at −40° C. and stirred vigorously at 25° C. for 10 min. The mixture was diluted with DCM (1 L), and then filtered and the solids were washed with DCM (500 mL×3). The combined filtrate was washed with brine (1 L), dried over Na₂SO₄, filtered and concentrated in vacuo to give the title compound as a solution in DCM/toluene (3/2, 2 L). The solution was used directly without further purification assuming quantitative yield. ¹H NMR (400 MHz, CDC₃) 5 9.59 (s, 1H), 2.15-2.01 (m, 2H), 1.80-1.75 (m, 4H), 1.55-1.45 (m, 2H), 1.25 (s, 3H).

Step 1C. (S,E)-2-Methyl-N-((1-methylcyclopentyl)methylene)propane-2-sulfinamide: To a solution of 1-methylcyclopentane-1-carbaldehyde (30.8 g, 275 mmol) in DCM/toluene (3/2, 2.0 L) at 20° C. was added titanium (IV) ethoxide (163 g, 717 mmol). The reaction was stirred for 20 min, and then (S)-2-methylpropane-2-sulfinamide (33.3 g, 275 mmol) was added. The resulting mixture was stirred at ambient temperature for 16 h. The reaction was quenched with water (250 mL). The mixture was filtered and the solids were washed with THF (3 L×3). The combined organic layers were concentrated in vacuo. The crude product was purified by silica gel column chromatography (100% petroleum ether) to afford 44.89 g (38%) of the title compound as a light yellow oil. LCMS m/z 216.3 [M+H]⁺. ¹H NMR (400 MHz, CDC₃) 6 7.94 (s, 1H), 1.93-1.85 (m, 2H), 1.80-1.60 (m, 4H), 1.55-1.45 (m, 2H), 1.21 (s, 3H), 1.20 (s, 9H).

Step 1D. 3-Iodo-1,4-dimethyl-1H-pyrazole: Route A) 3-Iodo-1,4-dimethyl-1H-pyrazole. To a solution of 1,4-dimethyl-1H-pyrazol-3-amine (1.0 g, 8.6 mmol) in conc. HCl (7.15 mL) at 0° C. was added a solution of sodium nitrite (1.22 g, 17 mmol) in water (1.78 mL) over 5 min. A solution of potassium iodide (3.57 g, 21.5 mmol) in water (3.6 mL) was then added dropwise over 5 min. The mixture was stirred at 0° C. for 30 min, and then warmed to ambient temperature and stirred for 2 h. The reaction mixture was diluted with THF (8 mL) and water (8 mL), and extracted with EtOAc (30 mL×4). The combined organic extracts were washed with saturated aqueous Na₂S₂O₃ solution (30 mL×2) followed by water (30 mL) and brine (30 mL), dried over Na₂SO₄, filtered and concentrated in vacuo. The crude product was purified by silica gel column chromatography (0 to 60% EtOAc in heptane) to afford 988 mg (52%) of the title compound as a white solid. LCMS m/z 223.0 [M+H]⁺. ¹H NMR (400 MHz, CDC₃) 6 7.05 (s, 1H), 3.87 (s, 3H), 1.98 (s, 3H).

Route B) Step 1. 3-Iodo-1-methyl-1H-pyrazole-4-carbaldehyde. POCl₃ (45.0 mL, 481 mmol) was added to a solution of 3-iodo-1-methyl-1H-pyrazole (25 g, 120.2 mmol) in DMF (150 mL) at 0° C. After 10 min, the reaction was heated to 65° C. for 18 h. A solution of NaH₂PO₄ (50 g in 200 mL) was added to the reaction slowly, ensuring the temperature remained between 25-35° C. and pH no greater than 4. After the addition, the reaction was stirred at room temperature for 45 min and then made basic by careful addition of saturated Na₂CO₃. The aqueous mixture was extracted with EtOAc. The organic layer was washed with brine, dried and filtered. The filtrate was concentrated. The resulting yellow oil was recrystallized from EtOAc/heptane to provided 28.4 g (88%) of the title compound as a tan solid. ¹H NMR (400 MHz, CDC₃) δ 9.67 (s, 1H), 7.84 (s, 1H), 4.00 (s, 4H).

Step 2. 3-Iodo-1,4-dimethyl-1H-pyrazole. Triethylsilane (5.08 mL, 0.32 mmol) and TFA (1.36 mL, 0.18 mmol) were added to a mixture of 3-iodo-1-methyl-1H-pyrazole-4-carbaldehyde 5 (1.0 g, 0.042 mmol) in chlorobenzene (10 mL). The mixture was heated to 50° C. overnight. The reaction mixture was cooled to room temperature and saturated aqueous NaHCO₃ was added. The mixture was extracted with EtOAc. The organic layer was washed with brine, dried (Na₂SO₄) and filtered. The filtrate was concentrated and the residue was purified by silica gel column chromatography (EtOAc/heptane) to afford 0.64 g (68%) of the title compound. ¹H NMR (400 MHz, CDC₃) 6 7.08 (s, 1H), 3.90 (s, 3H), 2.01 (s, 3H).

Route C) Step 1: 3-Iodo-4-methyl-1H-pyrazole. NIS (2196.0 g, 9.76 mol) was added in portions to a solution of 4-methyl-1H-pyrazole (800.0 g, 9.76 mol) in DMF (5 L) maintaining the temperature below 25° C. The mixture was stirred at 25° C. for 20 h. Two batches were combined. The combined mixtures were poured into water (20 L) and then extracted with MTBE (5 L×5). The combined organic layers were washed with brine (5 L×3), dried over Na₂SO₄ and filtered. The filtrate was concentrated in vacuo, and the residue was triturated with petroleum ether/EtOAc (5 L, 10:1). The suspension was stirred at 16° C. for 2 h and then filtered. The resulting solid was dried in vacuo to afford 1280.0 g (32%) of the title compound as a light yellow solid. ¹H NMR (CDC₃, 400 MHz) δ 7.42 (s, 1H), 2.07 (s, 3H).

Step 2. 3-iodo-1,4-dimethyl-1H-pyrazole. To a suspension of 60% NaH in mineral oil (165.0 g, 4.12 mol) in anhydrous THF (4.5 L) was added a mixture of 3-iodo-4-methyl-1H-pyrazole (710.0 g, 3.41 mol) in anhydrous THF (1.5 L) dropwise at 0° C. The mixture was stirred at 10° C. for 1 h. Iodomethane (496.0 g, 3.49 mol) was added dropwise to the mixture at 0° C. The resulting mixture was stirred at 20° C. for 16 h. The reaction was quenched with water (4.5 L) and extracted with EtOAc (2.5 L×3). The combined organic layers were dried over Na₂SO₄ and filtered. The filtrate was concentrated in vacuo. The residue was purified by silica gel column chromatography (petroleum ether:EtOAc=10:1 to 1:1) to give two fractions. The first fraction (150 g) was diluted with petroleum ether (200 mL) and stirred at 10° C. for 30 min. The suspension was filtered. The solid was washed with petroleum ether (100 mL) and dried in vacuo to give 100.0 g of the title compound as a white solid. The filtrate was concentrated in vacuo, combined with the second fraction (1300 g), and purified by silica gel column chromatography (petroleum ether: EtOAc=10:1 to 1:1) to afford additional desired compound (500.0 g). This compound was diluted with petroleum ether (800 mL) and stirred at 10° C. for 30 min. The suspension was filtered. The filter cake washed with petroleum ether (500 mL) and dried in vacuo to give 440.0 g of the title compound as a white solid. The total yield was 540 g (35.6%). LCMS m/z 222.8 [M+H]⁺. ¹H NMR (CDC₃, 400 MHz) δ 7.04 (s, 1H), 3.86 (s, 3H), 1.97 (s, 3H).

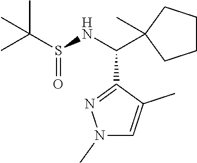 [SMILES: Cc1cn(C)nc1[C@H](N[S@@](=O)C(C)(C)C)C1(C)CCCC1]

Step 1E. (S)—N—((R)-(1,4-Dimethyl-1H-pyrazol-3-yl)(1-methylcyclopentyl)methyl)-2-methylpropane-2-sulfinamide: 3-Iodo-1,4-dimethyl-1H-pyrazole (33 g, 148.6 mmol) in THF (50 mL) was added over 1 h to a solution of 1.0 M iPrMgCl—LiCl in THF (189 mL, 189 mmol) maintaining an internal temperature at 0-5° C. under nitrogen. After 1 h, (S,E)-2-methyl-N-((1-methylcyclopentyl)methylene)propane-2-sulfinamide (20 g, 92.85 mmol) in THF (50 mL) was added and the mixture stirred at 25° C. for 18 h. The reaction was cooled to 0° C. and 10% acetic acid was added. The organic layer was separated and was partially concentrated. MTBE and water were added. The mixture was stirred for 10 min and the layers separated. The organic layer was concentrated to afford the title compound (66% purity) which was used in the subsequent step without further purification.

Step 1F. (R)-(1,4-Dimethyl-1H-pyrazol-3-yl)(1-methylcyclopentyl)methanamine: The crude (S)—N—((R)-(1,4-dimethyl-1H-pyrazol-3-yl)(1-methylcyclopentyl)methyl)-2-methylpropane-2-sulfinamide (231 g) was suspended in MTBE (300 mL), cooled to 10° C., and conc. HCl (38.4 mL, 2 eq) was added. The mixture was stirred at 10-20° C. for 1 h and then was diluted with water (300 mL). The layers were separated and the organic layer discarded. The aqueous layer was made basic with 20% NaOH to pH 11-13 and extracted with MTBE (2×300 mL). The combined organic layers were concentrated to afford 120 g of the title compound as an oil. The absolute stereochemistry of the title compound was assigned based on the open transition state analysis as described in the literature (Robak, M. T.; Herbage, M. A. Ellman, J. A. Chem. Rev. 2010, 110, 3600) and confirmed definitively by Mosher's amide analysis.

Step 1G. (R)-(1,4-Dimethyl-1H-pyrazol-3-yl)(1-methylcyclopentyl)methanamine L-pyroglutamic acid salt: L-pyroglutamic acid (77.7 g, 0.6 mol) was added to a solution of (R)-(1,4-dimethyl-1H-pyrazol-3-yl)(1-methylcyclopentyl)methanamine (100 g, 0.48 mol) in THF (1.3 L) at 10-20° C. The mixture was heated to 50° C. and stirred for 2 h, then cooled to 25° C. over 18 h.

The solid was filtered and rinsed with THF (890 mL). The solid was dried under vacuum at 45° C. for 6 h to afford 259.6 g (88%) of the title compound. ¹H NMR (400 MHz, DMSO-d₆) δ 7.58 (br. s., 1H), 7.40 (s, 1H), 3.93 (s, 1H), 3.87-3.84 (m, 1H), 3.77 (s, 3H), 2.27-2.14 (m, 1H), 2.12-2.02 (m, 2H), 1.98 (s, 3H), 1.96-1.84 (m, 1H), 1.70-1.48 (m, 6H), 1.42-1.31 (m, 1H), 1.08-1.02 (m, 1H), 0.97 (s, 3H). Chiral SFC (SFC method M) RT=3.81 min, 99% ee.

Step 1H. 4-((2-Ethoxy-3,4-dioxocyclobut-1-en-1-yl)amino)-3-hydroxy-N,N-dimethylpicolinamide: 4-((2-Ethoxy-3,4-dioxocyclobut-1-en-1-yl)amino)-3-hydroxy-N,N-dimethylpicolinamide was prepared as described in WO/2010/131145.

Step 1I. (R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide: A mixture of 4-((2-ethoxy-3,4-dioxocyclobut-1-en-1-yl)amino)-3-hydroxy-N,N-dimethylpicolinamide (180 g, 590 mmol), (R)-(1,4-dimethyl-1H-pyrazol-3-yl)(1-methylcyclopentyl)methanamine L-pyroglutamic acid salt (209 g, 625 mmol), EtOH (1 L) and DIPEA (205 mL, 1180 mmol) was stirred at 20-30° C. for 2h. HOAc (23.8 mL, 416 mmol) was added to adjust the pH to 6-7. The reaction mixture was then concentrated under reduced pressure to about half the volume. The mixture was diluted with water (3.2 L) and stirred for 1.5 hr. The resulting solid was filtered, washed with water and then dried under vacuum at 40-50° C. for 20 h to provide 256 g (93%) of the title compound as a yellow solid. LCMS m/z 467.4 [M+H]⁺. ¹H NMR (400 MHz, DMSO-d₆) δ 11.59 (br. s., 1H), 9.92 (br. s., 1H), 9.16 (s, 1H), 8.03-8.00 (m, 2H), 7.43 (s, 1H), 5.33 (d, J=10.0 Hz, 1H), 3.80 (s, 3H), 3.18 (s, 3H), 3.05 (s, 3H), 2.00 (s, 3H), 1.80-1.50 (m, 6H), 1.20-1.15 (m, 1H), 1.14-1.09 (m, 1H), 1.08 (s, 3H). [α]²⁰_{D}=−78.4 (c=1.0, MeOH). Chiral SFC (SFC method N) RT=4.77 min, 98.5% ee.

The title compounds in Table 1 were prepared in a similar manner to (R)-4-((2-(((1,4-dimethyl-1H-pyrazol-3-yl)(1-methylcyclopentyl)methyl)amino)-3,4-dioxocyclobut-1-en-1-yl)amino)-3-hydroxy-N,N-dimethylpicolinamide (Preparative Step 11) from the appropriate amine and a 4-((2-ethoxy-3,4-dioxocyclobut-1-en-1-yl)amino)-3-hydroxy-N,N-dialkylylpicolinamide.

TABLE 1

| Ex. | Structure | Chemical Name and Analytical Data |
|---|---|---|
| 1 | 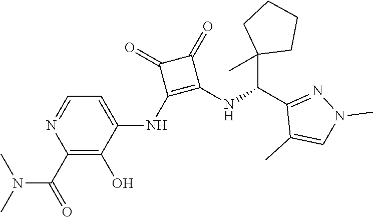 | (R)-4-((2-(((1,4-Dimethyl-1H-pyrazol-3-yl)(1- methylcyclopentyl)methyl)amino)-3,4-dioxocyclobut-1- en-1-yl)amino)-3-hydroxy-N,N-dimethylpicolinamide; LCMS m/z 467.4 [M + H]⁺. ¹H NMR (400 MHz, DMSO-d₆) δ 11.59 (br. s., 1H), 9.92 (br. s., 1H), 9.16 (s, 1H), 8.03- 8.00 (m, 2H), 7.43 (s, 1H), 5.33 (d, J = 10.0 Hz, 1H), 3.80 (s, 3H), 3.18 (s, 3H), 3.05 (s, 3H), 2.00 (s, 3H), 1.80-1.50 (m, 6H), 1.20-1.15 (m, 1H), 1.14-1.09 (m, 1H), 1.08 (s, 3H). [α]²⁰_{D} = −78.4 (c = 1.0, MeOH). Chiral SFC (SFC method N) RT = 4.77 min, 98.5% ee. |
| 2 | 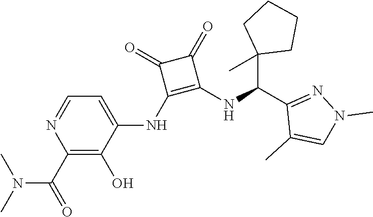 | (S)-4-((2-(((1,4-Dimethyl-1H-pyrazol-3-yl)(1- methylcyclopentyl)methyl)amino)-3,4-dioxocyclobut-1- en-1-yl)amino)-3-hydroxy-N,N-dimethylpicolinamide; LCMS m/z 467.4 [M + H]⁺. ¹H NMR (400 MHz, DMSO-d₆) δ 9.18 (br. s., 1H), 8.12-7.85 (m, 2H), 7.43 (s, 1H), 5.34 (d, J = 10.0 Hz, 1H), 3.80 (s, 3H), 3.15 (br. s., 3H), 3.05 (br. s., 3H), 1.98 (s, 3H), 1.74-1.52 (m, 6H), 1.41-1.27 (m, 1H), 1.23-1.15 (m, 1H), 1.08 (s, 3H). [α]²⁹_{D} = +88.49 (c = 0.5, MeOH). Chiral SFC (SFC method F) RT = 5.45 min, 100% ee. |
| 3 | 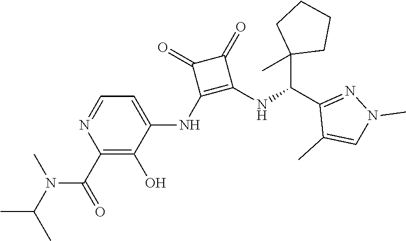 [SMILES: Cc1cn(C)nc1[C@H](Nc1c(Nc2ccnc(C(=O)N(C)C(C)C)c2O)c(=O)c1=O)C1(C)CCCC1] | (R)-4-((2-(((1,4-Dimethyl-1H-pyrazol-3-yl)(1- methylcyclopentyl)methyl)amino)-3,4-dioxocyclobut-1- en-1-yl)amino)-3-hydroxy-N-isopropyl-N- methylpicolinamide; LCMS m/z 495.5 [M + H]⁺. ¹H NMR (400 MHz, DMSO-d₆) δ 11.35 (s, 0.3H), 10.85 (s, 0.7H), 9.88 (s, 1H), 9.11 (d, J = 9.8 Hz, 1H), 8.05-7.90 (m, 2H), 7.43 (s, 1H), 5.33 (d, J = 10.0 Hz, 1H), 4.81 (s, 0.3H), 4.20 (s, 0.7H), 3.80 (s, 3H), 2.88 (s, 3H), 1.98 (s, 3H), 1.75-1.51 (m, 6H), 1.36- 1.32 (m, 1H), 1.25-1.10 (m, 7H), 1.08 (s, 3H). [α]²⁰_{D} = −49.8 (c = 0.26, MeOH). Chiral SFC conditions (SFC method D) RT = 4.62 min, 100% ee. |
| 4 | 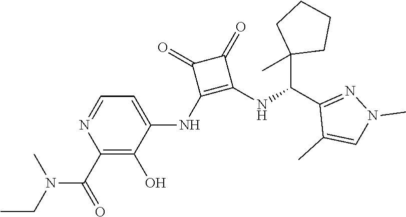 [SMILES: CCN(C)C(=O)c1nccc(Nc2c(N[C@@H](c3nn(C)cc3C)C3(C)CCCC3)c(=O)c2=O)c1O] | (R)-4-((2-(((1,4-Dimethyl-1H-pyrazol-3-yl)(1- methylcyclopentyl)methyl)amino)-3,4-dioxocyclobut-1- en-1-yl)amino)-N-ethyl-3-hydroxy-N-methylpicolinamide; LCMS m/z 481.1 [M + H]⁺. ¹H NMR (400 MHz, CD₃OD) δ 8.22 (br. s., 1H), 7.94 (br. s., 1H), 7.28 (s, 1H), 5.44 (s, 1H), 3.83 (s, 3H), 3.65 (br. s., 2H), 3.28-3.06 (m, 3H), 2.09 (s, 3H), 1.92-1.58 (m, 6H), 1.53-1.42 (m, 1H), 1.38-1.21 (m, 4H), 1.17 (s, 3H). [α]²⁶_{D} = −77.88 (c = 0.5, MeOH). Chiral SFC (SFC method F) RT = 3.52 min, 99% ee. |
| 5 | 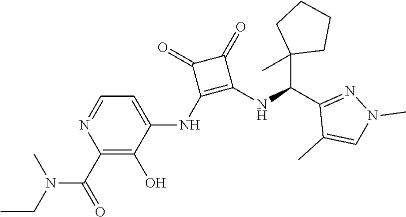 [SMILES: CCN(C)C(=O)c1nccc(Nc2c(N[C@H](c3nn(C)cc3C)C3(C)CCCC3)c(=O)c2=O)c1O] | (S)-4-((2-(((1,4-Dimethyl-1H-pyrazol-3-yl)(1- methylcyclopentyl)methyl)amino)-3,4-dioxocyclobut-1- en-1-yl)amino)-N-ethyl-3-hydroxy-N-methylpicolinamide; LCMS m/z 481.4 [M + H]⁺. ¹H NMR (400 MHz, DMSO-d₆) δ 11.70 (br. s., 1H), 9.90 (br. s., 1H), 9.15 (br. s., 1H), 8.00 (br. s., 2H), 7.43 (s, 1H), 5.34 (d, J = 10.0 Hz, 1H), 3.80 (s, 3H), 3.54 (br. s., 2H), 3.17 (br. s., 1H), 3.02 (br. s., 2H), 1.98 (s, 3H), 1.75-1.52 (m, 6H), 1.40-1.28 (m, 1H), 1.25-1.10 (4H), 1.08 (s, 3H). [α]²⁹_{D} = +80.8 (c = 0.5, MeOH). Chiral SFC (SFC method K) RT = 3.11 min, 99% ee. |
| 6 | 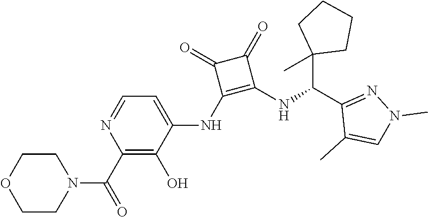 [SMILES: Cc1cn(C)nc1[C@H](Nc1c(Nc2ccnc(C(=O)N3CCOCC3)c2O)c(=O)c1=O)C1(C)CCCC1] | (R)-3-(((1,4-dimethyl-1H-pyrazol-3-yl)(1- methylcyclopentyl)methyl)amino)-4-((3-hydroxy-2- (morpholine-4-carbonyl)pyridin-4-yl)amino)cyclobut-3- ene-1,2-dione; LCMS m/z 509.4 [M + H]⁺. ¹H NMR (400 MHz, CD₃OD) δ 8.22 (d, J = 5.5 Hz, 1H), 7.91 (br s, 1H), 7.28 (s, 1H), 5.44 (s, 1H), 3.90-3.70 (m, 11H), 2.09 (s, 3H), 1.90-1.65 (m, 6H), 1.50-1.43 (m, 1H), 1.35-1.26 (m, 1H), 1.17 (s, 3H). [α]²⁰_{D} = −65.6 (c = 0.17, MeOH). Chiral SFC conditions (SFC method K) RT = 3.47 min, 100% ee. |
| 7 | 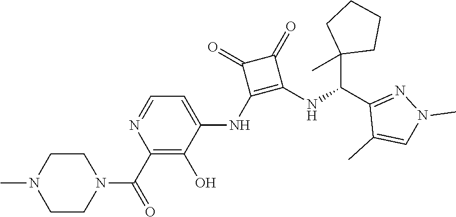 | (R)-3-(((1,4-Dimethyl-1H-pyrazol-3-yl)(1- methylcyclopentyl)methyl)amino)-4-((3-hydroxy-2-(4- methylpiperazine-1-carbonyl)pyridin-4- yl)amino)cyclobut-3-ene-1,2-dione; LCMS m/z 522.5 [M + H]⁺. ¹H NMR (400 MHz, CD₃OD) δ 8.35 (s, 1H), 8.24 (d, J = 5.6 Hz, 1H), 7.90 (d, J = 5.6 Hz, 1H), 7.28 (s, 1H), 5.44 (s, 1H), 3.92 (br s, 4H), 3.82 (s, 3H), 2.80 (br s, 4H), 2.53 (s, 3H), 2.09 (s, 3H), 1.90- 1.65 (m, 6H), 1.51-1.42 (m, 1H), 1.35-1.25 (m, 1H), 1.17 (s, 3H). Chiral SFC (SFC method F) RT = 3.66 min, 98.6% ee. |
| 8 | 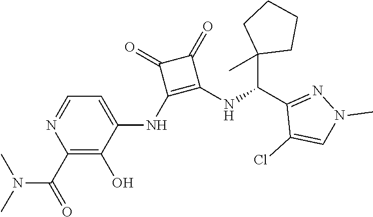 | (R)-4-((2-(((4-Chloro-1-methyl-1H-pyrazol-3-yl)(1- methylcyclopentyl)methyl)amino)-3,4-dioxocyclobut-1- en-1-yl)amino)-3-hydroxy-N,N-dimethylpicolinamide; LCMS m/z 487.3 [M + H]⁺. ¹H NMR (400 MHz, DMSO-d₆) δ 11.67 (br s, 1H), 9.95 (br s, 1H), 9.18 (s, 1H), 8.01 (s, 2H), 7.95 (s, 1H), 5.44 (d, J = 10.0 Hz, 1H), 3.86 (s, 3H), 3.18 (br s, 3H), 3.05 (br s, 3H), 1.78-1.58 (m, 6H), 1.39- 1.31 (m, 1H), 1.21-1.10 (m, 1H), 1.07 (s, 3H). [α]²⁴, = −145.151 (c = 0.50, MeOH). Chiral SFC (SFC method F) RT = 3.85 min, 100% ee. |
| 9 | 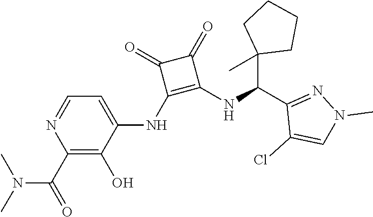 | (S)-4-((2-(((4-Chloro-1-methyl-1H-pyrazol-3-yl)(1- methylcyclopentyl)methyl)amino)-3,4-dioxocyclobut-1- en-1-yl)amino)-3-hydroxy-N,N-dimethylpicolinamide; LCMS m/z 487.4 [M + H]⁺. ¹H NMR (400 MHz, DMSO-d₆) δ 11.68 (br. s., 1H), 9.92 (br. s., 1H), 9.16 (br. s., 1H), 8.02-7.98 (m, 2H), 7.94 (s, 1H), 5.37 (d, J = 10.2 Hz, 1H), 3.86 (s, 3H), 3.18 (br. s., 3H), 3.05 (br. s., 3H), 1.74-1.55 (m, 6H), 1.37-1.30 (m, 1H), 1.21-1.14 (m, 1H), 1.07 (s, 3H). [α]²⁰_{D} = +107.3 [c = 1, MeOH). Chiral SFC (SFC method L) RT = 6.28 min, 98% ee. |
| 10 | 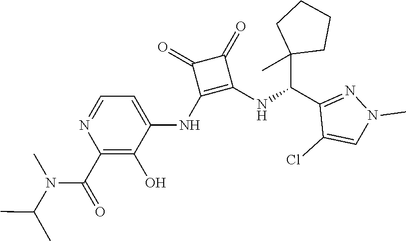 [SMILES: CC(C)N(C)C(=O)c1nccc(Nc2c(N[C@@H](c3nn(C)cc3Cl)C3(C)CCCC3)c(=O)c2=O)c1O] | (R)-4-((2-(((4-Chloro-1-methyl-1H-pyrazol-3-yl)(1- methylcyclopentyl)methyl)amino)-3,4-dioxocyclobut-1- en-1-yl)amino)-3-hydroxy-N-isopropyl-N- methylpicolinamide; LCMS m/z 515.3 [M + H]⁺. ¹H NMR (400 MHz, CD₃OD) δ 8.21 (d, J = 5.1 Hz, 1H), 7.93 (br s, 1H), 7.64 (s, 1H), 5.55 (s, 1H), 4.70-4.55 (m, 0.4H), 4.40-4.20 (m, 0.6H), 3.88 (s, 3H), 3.01 (s, 3H), 1.95-1.62 (m, 6H), 1.51-1.42 (m, 1H), 1.35-1.20 (m, 7H), 1.18 (s, 3H). [α]²⁰_{D} = −75.5 (c = 0.23, MeOH). Chiral SFC (SFC method B) RT = 1.59 min, 100% ee. |
| 11 | 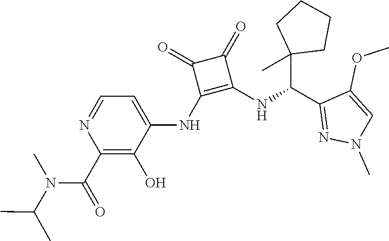 [SMILES: COc1cn(C)nc1[C@H](Nc1c(Nc2ccnc(C(=O)N(C)C(C)C)c2O)c(=O)c1=O)C1(C)CCCC1] | (R)-3-Hydroxy-N-isopropyl-4-((2-(((4-methoxy-1-methyl- 1H-pyrazol-3-yl)(1-methylcyclopentyl)methyl)amino)-3,4- dioxocyclobut-1-en-1-yl)amino)-N-methylpicolinamide; LCMS m/z 511.4 [M + H]⁺. ¹H NMR (400 MHz, DMSO-d₆) δ 11.34 (s, 0.3H), 10.84 (s, 0.7H), 9.94 (s, 1H), 9.13 (d, J = 9.8 Hz, 1H), 7.99-7.94 (m, 2H), 7.48 (s, 1H), 5.34 (d, J = 10.3 Hz, 1H), 4.82 (br s, 0.3H), 4.18 (br s, 0.7H), 3.78 (s, 3H), 3.67 (s, 3H), 2.95-2.84 (m, 3H), 1.72-1.50 (m, 6H), 1.35-1.24 (m, 1H), 1.21-1.10 (m, 7H), 1.02 (s, 3H). [α]²⁴_{D} = −114.4 (c = 0.50, MeOH). Chiral SFC (SFC method B) RT = 1.52 min, 100% ee. |
| 12 | 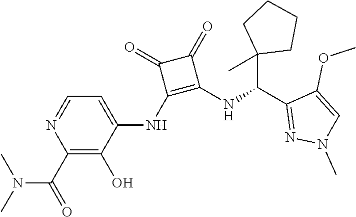 | (R)-3-Hydroxy-4-((2-(((4-methoxy-1-methyl-1H-pyrazol- 3-yl)(1-methylcyclopentyl)methyl)amino)-3,4- dioxocyclobut-1-en-1-yl)amino)-N,N- dimethylpicolinamide; LCMS m/z 483.4 [M + H]⁺. ¹H NMR (400 MHz, CD₃OD) δ 8.21 (d, J = 5.5 Hz, 1H), 7.90 (d, J = 5.5 Hz, 1H), 7.30 (s, 1H), 5.43 (s, 1H), 3.78 (s, 3H), 3.74 (s, 3H), 3.24 (br s, 3H), 3.17 (br s, 3H), 1.91-1.74 (m, 2H), 1.70-1.60 (m, 4H), 1.45-1.35 (m, 1H), 1.30-1.20 (m, 1H), 1.11 (s, 3H). [α]²⁴_{D} = −87.5 (c = 0.50, MeOH). Chiral SFC (SFC method F) RT = 4.86 min, 97.4% ee. |
| 13 | 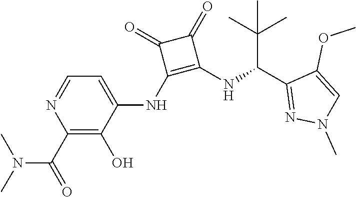 | (R)-3-Hydroxy-4-((2-((1-(4-methoxy-1-methyl-1H- pyrazol-3-yl)-2,2-dimethylpropyl)amino)-3,4- dioxocyclobut-1-en-1-yl)amino)-N,N- dimethylpicolinamide; LCMS m/z 457.4 [M + H]⁺. ¹H NMR (400 MHz, DMSO-d₆) δ 11.64 (br s, 1H), 9.99 (s, 1H), 9.12 (d, J = 8.8 Hz, 1H), 8.01 (s, 2H), 7.49 (s, 1H), 5.22 (d, J = 10.3 Hz, 1H), 3.79 (s, 3H), 3.67 (s, 3H), 3.19 (br s, 3H), 3.06 (br s, 3H), 0.94 (s, 9H). [α]²⁴_{D} = −110.8 (c = 0.33, MeOH). Chiral SFC (SFC method J) RT = 3.95 min, 100% ee. |
| 14 | 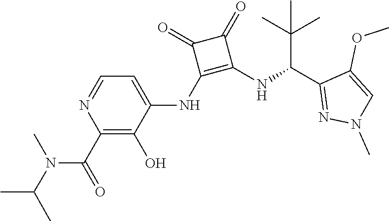 [SMILES: COc1cn(C)nc1[C@H](Nc1c(Nc2ccnc(C(=O)N(C)C(C)C)c2O)c(=O)c1=O)C(C)(C)C] | (R)-3-Hydroxy-N-isopropyl-4-((2-((1-(4-methoxy-1- methyl-1H-pyrazol-3-yl)-2,2-dimethylpropyl)amino)-3,4- dioxocyclobut-1-en-1-yl)amino)-N-methylpicolinamide; LCMS m/z 485.4 [M + H]⁺. ¹H NMR (400 MHz, CD₃OD) δ 8.18 (br s, 1H), 7.94 (br s, 1H), 7.33 (s, 1H), 5.35 (s, 1H), 4.64 (br s, 0.3H), 4.30 (br s, 0.7H), 3.81 (s, 3H), 3.76 (s, 3H), 3.02 (s, 3H), 1.33-1.20 (m, 6H), 1.04 (s, 9H). Chiral SFC (SFC method K) RT = 2.78 min, 98.7% ee. |
| 15 | 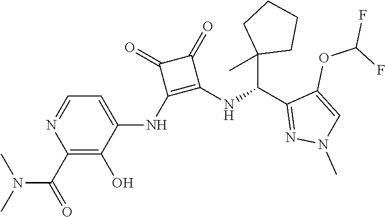 [SMILES: CN(C)C(=O)c1nccc(Nc2c(N[C@@H](c3nn(C)cc3OC(F)F)C3(C)CCCC3)c(=O)c2=O)c1O] | (R)-4-((2-(((4-(Difluoromethoxy)-1-methyl-1H-pyrazol-3- yl)(1-methylcyclopentyl)methyl)amino)-3,4- dioxocyclobut-1-en-1-yl)amino)-3-hydroxy-N,N- dimethylpicolinamide; LCMS m/z 519.3 [M + H]⁺. ¹H NMR (400 MHz, CD₃OD) δ 8.22 (d, J = 5.5 Hz, 1H), 7.92 (br s, 1H), 7.57 (s, 1H), 6.65 (dd, J = 75.5, 72.9 Hz, 1H), 5.46 (s, 1H), 3.86 (s, 3H), 3.24 (br s, 3H), 3.17 (br s, 3H), 1.87-1.62 (m, 6H), 1.51-1.43 (m, 1H), 1.33-1.25 (m, 1H), 1.15 (s, 3H). 19F NMR (376 MHz, CDCl₃) δ −85.1. Chiral SFC (SFC method B) RT = 1.34 min, 100% ee. |
| 16 | 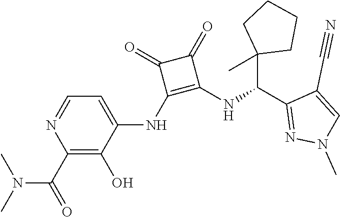 [SMILES: CN(C)C(=O)c1nccc(Nc2c(N[C@@H](c3nn(C)cc3C#N)C3(C)CCCC3)c(=O)c2=O)c1O] | (R)-4-((2-(((4-Cyano-1-methyl-1H-pyrazol-3-yl)(1- methylcyclopentyl)methyl)amino)-3,4-dioxocyclobut-1- en-1-yl)amino)-3-hydroxy-N,N-dimethylpicolinamide; LCMS m/z 478.4 [M + H]⁺. ¹H NMR (400 MHz, CD₃OD) δ 8.22 (d, J = 5.5 Hz, 1H), 8.19 (s, 1H), 7.95-7.85 (m, 1H), 5.52 (s, 1H), 3.95 (s, 3H), 3.24 (br s, 3H), 3.17 (br s, 3H), 1.95-1.65 (m, 6H), 1.55-1.45 (m, 1H), 1.44-1.35 (m, 1H), 1.15 (s, 3H). Chiral HPLC (HPLC method A) RT = 8.65 min, 98.0% ee. |
| 17 | 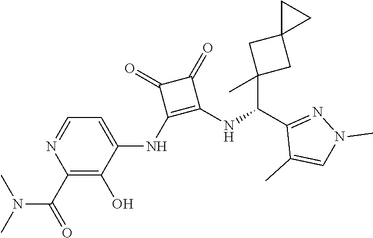 | (R)-4-((2-(((1,4-Dimethyl-1H-pyrazol-3-yl)(5- methylspiro[2.3]hexan-5-yl)methyl)amino)-3,4- dioxocyclobut-1-en-1-yl)amino)-3-hydroxy-N,N- dimethylpicolinamide; LCMS m/z 479.8 [M + H]⁺. ¹H NMR (400 MHz, DMSO-d₆) δ 9.93 (br s, 1H), 9.18 (br s, 1H), 8.04-8.00 (m, 2H), 7.44 (s, 1H), 5.53 (d, J = 10.1, 1H), 3.79 (s, 3H), 3.20 (br s, 3H), 3.07 (br s, 3H), 2.28 (d, J = 11.7, 1H), 2.08 (d, J = 11.3, 1H), 2.01 (s, 3H), 1.73 (d, J = 11.3 Hz, 1H), 1.66 (d, J = 10.5 Hz, 1H), 1.35 (s, 3H), 0.45-0.28 (m, 4H). Chiral SFC conditions (Chiral Tech OD-H 21.2 × 21.2 mm I.D. 5 μm, 45% (0.2% 7N ammonia in MeOH) in EtOH, 80 mL/min) RT = 5.39 min, 98.8% ee (title compound) and RT = 5.98 min, 96% ee (enantiomer). |
| 18 | 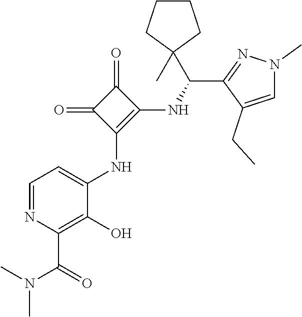 | (R)-4-((2-(((4-Ethyl-1-methyl-1H-pyrazol-3-yl)(1- methylcyclopentyl)methyl)amino)-3,4-dioxocyclobut-1- en-1-yl)amino)-3-hydroxy-N,N-dimethylpicolinamide. Yellow solid, 42.8 mg (34%). LCMS m/z 481.4 [M + H]⁺. ¹H NMR (400 MHz, CD₃OD) δ 8.25-8.22 (m, 1H), 7.95- 7.85 (m, 1H), 7.33 (s, 1H), 5.44 (s, 1H), 3.85 (s, 3H), 3.23 (br s, 3H), 3.16 (br s, 3H), 2.65-2.45 (m, 2H), 1.90- 1.60 (m, 6H), 1.50-1.40 (m, 1H), 1.40-1.30 (m, 1H), 1.20 (t, J = 7.5 Hz, 3H), 1.17 (s, 3H). [α]²⁴_{D} = −28.65 (c 1.00, MeOH). RT = 1.56 min, 99.2% ee (SFC method B) |
| 19 | 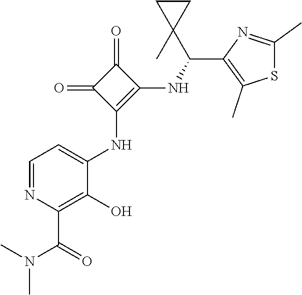 | (R)-4-((2-(((2,5-Dimethylthiazol-4-yl)(1- methylcyclopropyl)methyl)amino)-3,4-dioxocyclobut-1- en-1-yl)amino)-3-hydroxy-N,N-dimethylpicolinamide. Yellow solid, 75.7 mg (41%). LCMS m/z 456.1 [M + H]⁺. ¹H NMR (400 MHz, CD₃OD) δ 8.21 (d, J = 5.6 Hz, 1H), 7.90 (br s, 1H), 5.32 (s, 1H), 3.21 (br s, 3H), 3.15 (br s, 3H), 2.60 (s, 3H), 2.40 (s, 3H), 1.21 (s, 3H), 0.80-0.75 (m, 1H), 0.50-0.45 (m, 1H), 0.43-0.3 (m, 2H). RT = 4.44 min, 100% ee (SFC method D) |
| 20 | 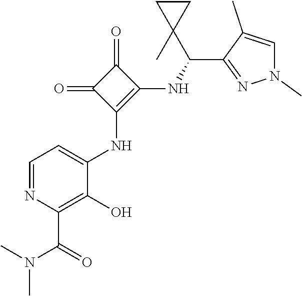 | (R)-4-((2-(((1,4-Dimethyl-1H-pyrazol-3-yl)(1- methylcyclopropyl)methyl)amino)-3,4-dioxocyclobut-1- en-1-yl)amino)-3-hydroxy-N,N-dimethylpicolinamide. Yellow solid, 193 mg (45%). LCMS m/z 439.3 [M + H]⁺. 1H NMR (400 MHz, CD₃OD) δ 8.22 (d, J = 5.4 Hz, 1H), 7.92 (br s, 1H), 7.29 (s, 1H), 5.24 (s, 1H), 3.80 (s, 3H), 3.23 (br s, 3H), 3.15 (br s, 3H), 2.06 (s, 3H), 1.21 (s, 3H), 0.82-0.74 (m, 1H), 0.48-0.38 (m, 3H). RT = 3.45 min, 100% ee (SFC method E) |
| 21 | 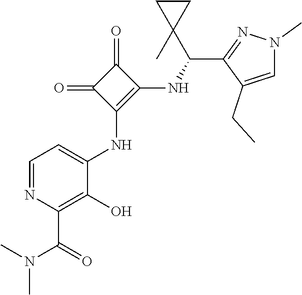 | (R)-4-((2-(((4-Ethyl-1-methyl-1H-pyrazol-3-yl)(1- methylcyclopropyl)methyl)amino)-3,4-dioxocyclobut-1- en-1-yl)amino)-3-hydroxy-N,N-dimethylpicolinamide. Yellow solid, 59 mg, (50%). LCMS m/z 453.3 [M + H]⁺. 1H NMR (400 MHz, CD₃OD) δ 8.22 (d, J = 5.5 Hz, 1H), 7.91 (br s, 1H), 7.31 (s, 1H), 5.27 (s, 1H), 3.80 (s, 3H), 3.23 (br s, 3H), 3.15 (br s, 3H), 2.55-2.40 (m, 2H), 1.21 (s, 3H), 1.20 (t, J = 7.6 Hz, 3H), 0.82-0.77 (m, 1H), 0.45- 0.37 (m, 3H). RT = 4.49 min, 94.1% ee (SFC method D) |
| 22 | 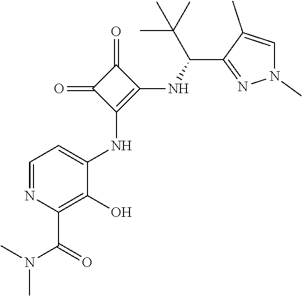 | (R)-4-((2-((1-(1,4-Dimethyl-1H-pyrazol-3-yl)-2,2- dimethylpropyl)amino)-3,4-dioxocyclobut-1-en-1- yl)amino)-3-hydroxy-N,N-dimethylpicolinamide. Yellow solid, 57 mg (44%). LCMS m/z 441.3 [M + H]⁺. ¹H NMR (400 MHz, CD₃OD) δ 8.20 (d, J = 5.5 Hz, 1H), 7.89 (d, J = 5.5 Hz, 1H), 7.26 (s, 1H), 5.31 (s, 1H), 3.81 (s, 3H), 3.21 (br s, 3H), 3.14 (br s, 3H), 2.06 (s, 3H), 1.05 (s, 9H). RT = 4.13 min, 99.1% ee (SFC method F) |
| 23 | 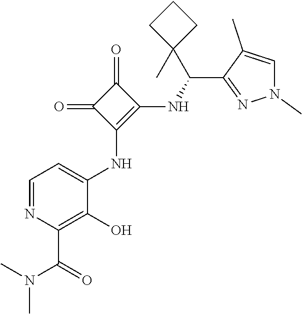 | (R)-4-((2-(((1,4-Dimethyl-1H-pyrazol-3-yl)(1- methylcyclobutyl)methyl)amino)-3,4-dioxocyclobut-1-en- 1-yl)amino)-3-hydroxy-N,N-dimethylpicolinamide. Yellow solid, 35 mg (34%). LCMS m/z 453.3 [M + H]⁺. ¹H NMR (400 MHz, CD₃OD) δ 8.22 (d, 5.5 Hz, 1H), 7.89 (d, J = 5.6 Hz, 1H), 7.29 (s, 1H), 5.45 (s, 1H), 3.82 (s, 3H), 3.22 (br s, 3H), 3.14 (br s, 3H), 2.28-2.17 (m, 2H), 2.07 (s, 3H), 2.02-1.88 (m, 1H), 1.85-1.59 (m, 3H), 1.32 (s, 3H). RT = 4.52 min, 99.7% ee (SFC method D) |
| 24 | 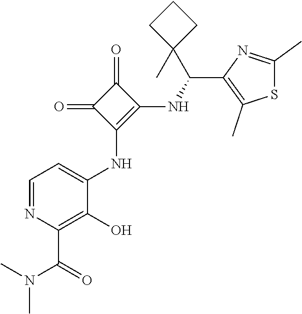 | (R)-4-((2-(((2,5-Dimethylthiazol-4-yl)(1- methylcyclobutyl)methyl)amino)-3,4-dioxocyclobut-1-en- 1-yl)amino)-3-hydroxy-N,N-dimethylpicolinamide. Yellow solid, 140 mg (34%). LCMS m/z 470.3 [M + H]⁺. ¹H NMR (400 MHz, DMSO-d₆) δ 11.69 (s, 1H), 9.98 (s, 1H), 9.21 (d, J = 9.9 Hz, 1H), 8.01 (s, 2H), 5.46 (d, J = 9.9 Hz, 1H), 3.20 (br s, 3H), 3.06 (br s, 3H), 2.62 (s, 3H), 2.40 (s, 3H), 2.19 (q, J = 9.8 Hz, 1H), 2.04 (q, J = 9.6 Hz, 1H), 1.96-1.83 (m, 1H), 1.80-1.69 (m, 1H), 1.65-1.51 (m, 2H), 1.19 (s, 3H). RT = 3.53 min, 97.2% ee (SFC method F) |
| 25 | 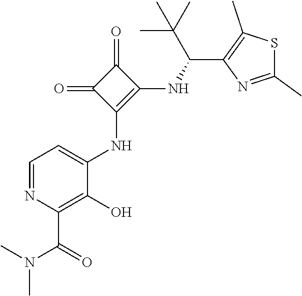 | (R)-4-((2-((1-(2,5-Dimethylthiazol-4-yl)-2,2- dimethylpropyl)amino)-3,4-dioxocyclobut-1-en-1- yl)amino)-3-hydroxy-N,N-dimethylpicolinamide. Yellow solid, 71 mg (18%). LCMS m/z 458.3 [M + H]⁺. ¹H NMR (400 MHz, DMSO-d₆) δ 11.74 (br s, 1H), 10.03 (br s, 1H), 9.23 (s, 1H), 8.10-7.88 (m, 2H), 5.30 (d, J = 10.1 Hz, 1H), 3.18 (s, 3H), 3.05 (s, 3H), 2.63 (s, 3H), 2.37 (s, 3H), 0.97 (s, 9H). RT = 3.23 min, 96.9% ee (SFC method F) |
| 26 | 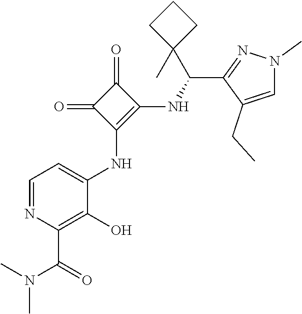 | (R)-4-((2-(((4-Ethyl-1-methyl-1H-pyrazol-3-yl)(1- methylcyclobutyl)methyl)amino)-3,4-dioxocyclobut-1-en- 1-yl)amino)-3-hydroxy-N,N-dimethylpicolinamide. Yellow solid, 23.5 mg (44%). LCMS m/z 467.4 [M + H]⁺. ¹H NMR (400 MHz, CD₃OD) δ 8.22 (d, J = 5.6 Hz, 1H), 7.90 (br s, 1H), 7.32 (s, 1H), 5.45 (s, 1H), 3.83 (s, 3H), 3.22 (br s, 3H), 3.15 (br s, 3H), 2.58-2.42 (m, 2H), 2.30-2.15 (m, 2H), 2.01-1.92 (m, 1H), 1.81-1.60 (m, 3H), 1.32 (s, 3H), 1.19 (t, J = 7.5 Hz, 3H). RT = 4.61 min, 100% ee (SFC method F) |
| 27 | 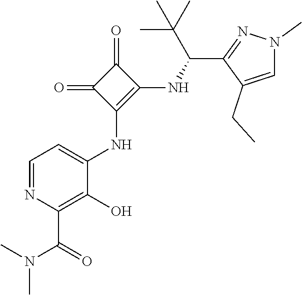 | (R)-4-((2-((1-(4-Ethyl-1-methyl-1H-pyrazol-3-yl)-2,2- dimethylpropyl)amino)-3,4-dioxocyclobut-1-en-1- yl)amino)-3-hydroxy-N,N-dimethylpicolinamide. Yellow solid, 41.4 mg (35%). LCMS m/z 455.4 [M + H]⁺. ¹H NMR (400 MHz, CD₃OD) δ 8.21 (d, J = 5.6 Hz, 1H), 7.91 (br s, 1H), 7.33 (s, 1H), 5.33 (s, 1H), 3.86 (s, 3H), 3.22 (br s, 3H), 3.14 (br s, 3H), 2.60-2.42 (m, 2H), 1.18 (t, J = 7.5 Hz, 3H), 1.07 (s, 9H). RT = 4.17 min, 95.7% ee (SFC method G) |
| 28 | 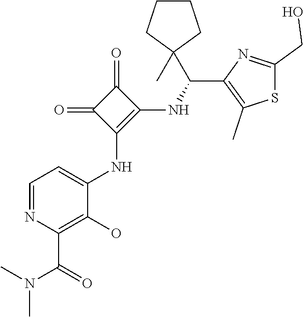 [SMILES: Cc1sc(CO)nc1[C@H](Nc1c(Nc2ccnc(C(=O)N(C)C)c2O)c(=O)c1=O)C1(C)CCCC1] | (R)-3-Hydroxy-4-((2-(((2-(hydroxymethyl)-5- methylthiazol-4-yl)(1-methylcyclopentyl)methyl)amino)- 3,4-dioxocyclobut-1-en-1-yl)amino)-N,N- dimethylpicolinamide. Yellow solid, 45.1 mg (46%) LCMS m/z 500.4 [M + H]⁺. ¹H NMR (400 MHz, CD₃OD) δ 8.21 (d, J = 5.6 Hz, 1H), 7.95-7.85 (m, 1H), 5.51 (s, 1H), 4.79 (s, 2H), 3.21 (br s, 3H), 3.14 (br s, 3H), 2.50 (s, 3H), 1.90-1.76 (m, 2H), 1.75-1.60 (m, 4H), 1.47-1.40 (m, 1H), 1.33-1.25 (m, 1H), 1.14 (s, 3H). RT = 5.15 min, 98.7% ee (SFC method G) |
| 29 | 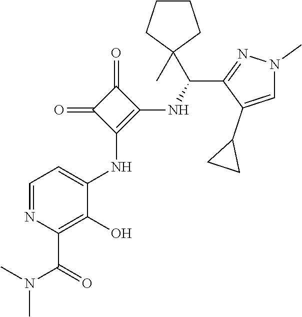 | (R)-4-((2-(((4-Cyclopropyl-1-methyl-1H-pyrazol-3-yl)(1- methylcyclopentyl)methyl)amino)-3,4-dioxocyclobut-1- en-1-yl)amino)-3-hydroxy-N,N-dimethylpicolinamide. Yellow solid, 15.9 mg (33%). LCMS m/z 493.4 [M + H]⁺. ¹H NMR (400 MHz, CD₃OD) δ 8.22 (d, J = 5.5 Hz, 1H), 7.95-7.85 (m, 1H), 7.14 (s, 1H), 5.63 (s, 1H), 3.81 (s, 3H), 3.32-3.11 (m, 6H), 1.98-1.75 (m, 2H), 1.75-1.60 (m, 5H), 1.53-1.42 (m, 1H), 1.39-1.30 (m, 1H), 1.20 (s, 3H), 0.89-0.86 (m, 2H), 0.54-0.52 (m, 1H), 0.44-0.41 (m, 1H). RT = 4.86 min, 100% ee (SFC method F) |
| 30 | 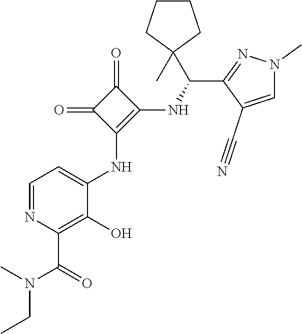 | (R)-4-((2-(((4-Cyano-1-methyl-1H-pyrazol-3-yl)(1- methylcyclopentyl)methyl)amino)-3,4-dioxocyclobut-1- en-1-yl)amino)-N-ethyl-3-hydroxy-N-methylpicolinamide. Yellow solid, 14.3 mg (23%). LCMS m/z 492.5 [M + H]⁺. ¹H NMR (400 MHz, CD₃OD) δ 8.22 (d, J = 5.5 Hz, 1H), 8.19 (s, 1H), 7.98-7.90 (m, 1H), 5.53 (s, 1H), 3.96 (s, 3H), 3.71-3.60 (m, 2H), 3.23-3.12 (m, 3H), 1.95-1.64 (m, 6H), 1.57-1.49 (m, 1H), 1.42-1.22 (m, 4H), 1.16 (s, 3H). RT = 5.03 min, 98.5% ee (SFC method H) |
| 31 | 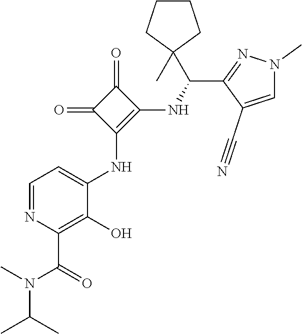 | (R)-4-((2-(((4-cyano-1-methyl-1H-pyrazol-3-yl)(1- methylcyclopentyl)methyl)amino)-3,4-dioxocyclobut-1- en-1-yl)amino)-3-hydroxy-N-isopropyl-N- methylpicolinamide. Yellow solid, 14.8 mg (24%). LCMS m/z 506.4 [M + H]⁺. ¹H NMR (400 MHz, CD₃OD) δ 8.24- 8.20 (m, 1H), 8.19 (s, 1H), 7.93 (br s, 1H), 5.53 (s, 1H), 4.35-4.25 (m, 1H), 3.96 (s, 3H), 3.02 (s, 3H), 1.95-1.65 (m, 6H), 1.60-1.45 (m, 1H), 1.40-1.15 (m, 7H), 1.16 (s, 3H). RT = 4.49 min, 97.6% ee (SFC method J) |
| 32 | 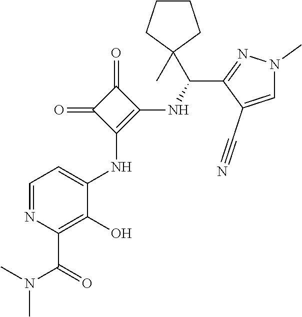 | (R)-4-((2-(((2,5-Dimethylthiazol-4-yl)(1- methylcyclopentyl)methyl)amino)-3,4-dioxocyclobut-1- en-1-yl)amino)-3-hydroxy-N,N-dimethylpicolinamide. Yellow solid, 63 mg (30%). LCMS m/z 484.4 [M + H]⁺. ¹H NMR (400 MHz, CD₃OD) δ 8.22 (d, J = 5.6 Hz, 1H), 7.92 (br s, 1H), 5.48 (s, 1H), 3.24 (br s, 3H), 3.16 (br s, 3H), 2.62 (s, 3H), 2.45 (s, 3H), 1.90-1.75 (m, 2H), 1.75-1.55 (m, 4H), 1.50-1.40 (m, 1H), 1.40-1.30 (m, 1H), 1.14 (s, 3H). RT = 3.44 min, 98.6% ee (SFC method F) |
| 33 | 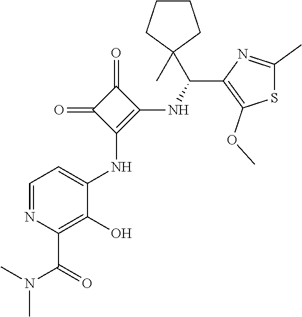 | (R)-3-Hydroxy-4-((2-(((5-methoxy-2-methylthiazol-4- yl)(1-methylcyclopentyl)methyl)amino)-3,4- dioxocyclobut-1-en-1-yl)amino)-N,N- dimethylpicolinamide. Yellow solid, 34.4 mg (42%). LCMS m/z 500.4 [M + H]⁺. ¹H NMR (400 MHz, CD₃OD) δ 8.22 (d, J = 5.6 Hz, 1H), 7.92 (br s, 1H), 5.46 (s, 1H), 3.94 (s, 3H), 3.22 (br s, 3H), 3.14 (br s, 3H), 2.58 (s, 3H), 1.88-1.76 (m, 2H), 1.70-1.60 (m, 4H), 1.43-1.38 (m, 1H), 1.32-1.25 (m, 1H), 1.12 (s, 3H). RT = 1.46 min, 100% ee (SFC method B) |
| 34 | 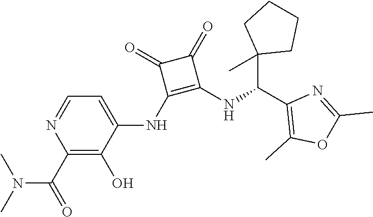 | (R)-4-((2-(((2,5-Dimethyloxazol-4-yl)(1- methylcyclopentyl)methyl)amino)-3,4-dioxocyclobut-1- en-1-yl)amino)-3-hydroxy-N,N-dimethylpicolinamide; LCMS m/z 468.6 [M + H]⁺. ¹H NMR (400 MHz, DMSO-d₆) δ 9.23-9.10 (m, 1H), 7.98-7.97 (m, 1H), 7.91 (br s, 1H), 5.17-5.15 (s, 1H), 3.17 (s, 3H), 3.04 (s, 3H), 2.37 (s, 3H), 2.25 (s, 3H), 1.78-1.47 (m, 7H), 1.37-1.10 (m, 3H), 1.05 (s, 3H). RT = 5.58 min, 100% ee (SFC method C) |
| 35 | 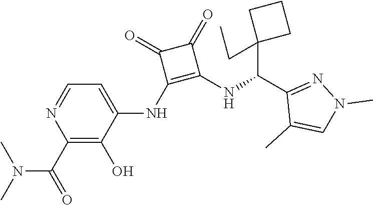 | (R)-4-((2-(((1,4-Dimethyl-1H-pyrazol-3-yl)(1- ethylcyclobutyl)methyl)amino)-3,4-dioxocyclobut-1-en-1- yl)amino)-3-hydroxy-N,N-dimethylpicolinamide; LCMS m/z 467.4 [M + H]⁺. ¹H NMR (400 MHz, CD₃OD) δ 8.27 (d, J = 5.6 Hz, 1H), 7.94 (d, J = 5.7 Hz, 1H), 7.31 (s, 1H), 5.51 (s, 1H), 3.84 (s, 3H), 3.30-3.10 (m, 6H), 2.46-2.38 (m, 1H), 2.15-2.05 (m, 4H), 1.95-1.60 (m, 6H), 1.00(t, J = 7.4, 3H). Chiral SFC (SFC method I) RT = 10.44 min, 97.2% ee (title compound) and RT = 14.45 min, 91.9% ee (enantiomer). |
| 36 | 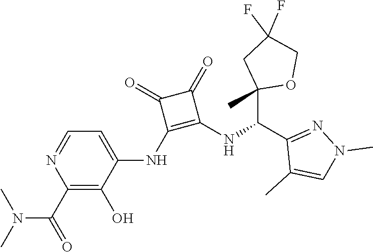 | 4-((2-(((S)-((S)-4,4-Difluoro-2-methyltetrahydrofuran-2- yl)(1,4-dimethyl-1H-pyrazol-3-yl)methyl)amino)-3,4- dioxocyclobut-1-en-1-yl)amino)-3-hydroxy-N,N- dimethylpicolinamide; LCMS m/z 505.4 [M + H]+ ¹H NMR (600 MHz, DMSO-d₆) δ 10.48 (br s, 2 H), 9.23 (br s, 1H), 7.99 (d, J=5.3 Hz, 1H), 7.91 (br. s., 1H), 7.49 (s, 1H), 5.56 (d, J = 8.4 Hz, 1H), 4.10-3.96 (m, 1H), 3.93-3.82 (m, 1H), 3.80 (s, 3H), 3.14 (br s, 3H), 3.03 (br s, 3H), 2.55-2.45 (m, 1H), 2.34- 2.22 (m, 1H), 1.99 (s, 3H), 1.49 (s, 3H). Chiral SFC (SFC method A) RT = 5.26 min, 100% ee. |
| 37 | 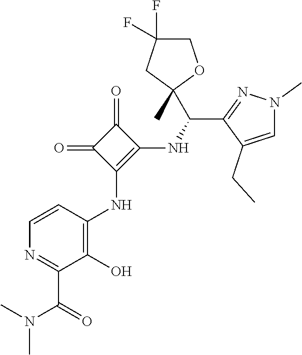 | 4-((2-(((S)-((S)-4,4-Difluoro-2-methyltetrahydrofuran-2- yl)(4-ethyl-1-methyl-1H-pyrazol-3-yl)methyl)amino)-3,4- dioxocyclobut-1-en-1-yl)amino)-3-hydroxy-N,N- dimethylpicolinamide. Yellow solid, 21.5 mg (15%). LCMS m/z 519.3 [M + H]⁺. ¹H NMR (400 MHz, CD₃OD) δ 8.24 (d, J = 5.6 Hz, 1H), 7.95-7.90 (m, 1H), 7.38 (s, 1H), 5.68 (s, 1H), 4.10-3.88 (m, 2H), 3.86 (s, 3H), 3.23 (br s, 3H), 3.16 (br s, 3H), 2.75-2.65 (m, 1H), 2.60-2.45 (m, 2H), 2.35-2.20 (m, 1H), 1.60 (s, 3H), 1.20 (t, J = 7.5 Hz, 3H). ¹⁹F NMR (376 MHz, CD₃OD) δ −104, −98.0. RT = 3.86 min, 100% ee (SFC method D) |
| 38 | 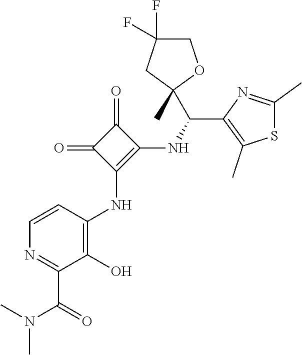 | 4-((2-(((S)-((S)-4,4-Difluoro-2-methyltetrahydrofuran-2- yl)(2,5-dimethylthiazol-4-yl)methyl)amino)-3,4- dioxocyclobut-1-en-1-yl)amino)-3-hydroxy-N,N- dimethylpicolinamide. Yellow solid, 22.9 mg (9%). LCMS m/z 522.3 [M + H]⁺. ¹H NMR (400 MHz, CD₃OD) δ 8.24 (d, J = 5.5 Hz, 1H), 7.92 (d, J = 5.5 Hz, 1H), 5.71 (s, 1H), 4.02 (q, J = 10.9 Hz, 1H), 3.86 (dt, J = 18.6, 10.3 Hz, 1H), 3.23 (br s, 3H), 3.16 (br s, 3H), 2.85-2.70 (m, 1H), 2.64 (s, 3H), 2.47 (s, 3H), 2.26 (ddd, J = 17.7, 14.4, 8.0 Hz, 1H), 1.54 (s, 3H). ¹⁹F NMR (376 MHz, CD₃OD) δ −104, −98. RT = 4.14 min, 100.0% ee (SFC method H) |
| 39 | 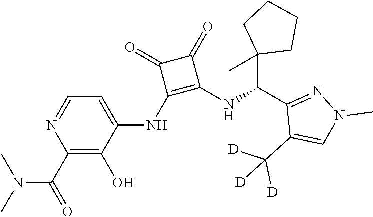 | (R)-3-hydroxy-N,N-dimethyl-4-((2-(((1-methyl-4-(methyl- d₃)-1H-pyrazol-3-yl)(1-methylcyclopentyl)methyl)amino)- 3,4-dioxocyclobut-1-en-1-yl)amino)picolinamide; LCMS m/z 470.5 [M + H]⁺. ¹H NMR (400 MHz, DMSO-d₆) δ 11.70 (s, 1H), 9.92 (s, 1H), 9.13 (d, J = 10.1 Hz, 1H), 8.02 (s, 2H), 7.44 (s, 1H), 5.35 (d, J = 10.1 Hz, 1H), 3.81 (s, 3H), 3.22-3.07 (m, 6H), 1.72-1.60 (m, 6H), 1.39- 1.12 (m, 2H), 1.10 (s, 3H). |
| 40 | 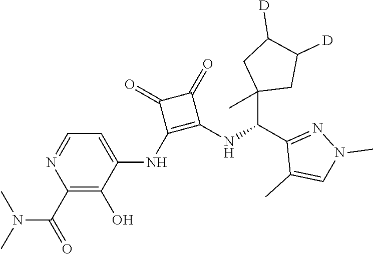 | 4-((2-(((1R)-(1,4-dimethyl-1H-pyrazol-3-yl)(1- methylcyclopentyl-3,4-d₂)methyl)amino)-3,4- dioxocyclobut-1-en-1-yl)amino)-3-hydroxy-N,N- dimethylpicolinamide; LCMS m/z 469.4 [M + H]⁺. ¹H NMR (400 MHz, DMSO d₆) δ 11.75 (br. s., 1H), 9.96 (br. s., 1H), 9.17 (br. s., 1H), 8.05-7.89 (m, 2H), 7.44 (s, 1H), 5.35 (d, J = 10.2 Hz, 1H), 3.81 (s, 3H), 3.20 (br. s., 3H), 3.07 (br. s., 3H), 2.00 (s, 3H), 1.72-1.55 (m, 4H), 1.37-1.31 (m, 1H), 1.25- 1.19 (m, 1H), 1.10 (s, 3H). |
| 41 | 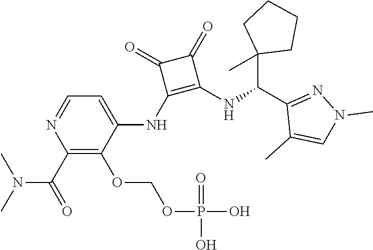 | (R)-((4-((2-(((1,4-Dimethyl-1H-pyrazol-3-yl)(1- methylcyclopentyl)methyl)amino)-3,4-dioxocyclobut-1- en-1-yl)amino)-2-(dimethylcarbamoyl)pyridin-3- yl)oxy)methyl dihydrogen phosphate; LCMS 577.5 [M + H]⁺. ¹H NMR (400 MHz, CD₃OD) δ 8.86 (d, J = 7.0 Hz, 1H), 8.49 (d, J = 6.6 Hz, 1H), 7.78 (s, 1H), 5.75-5.63 (m, 1H), 5.52-5.40 (m, 2H), 4.01 (s, 3H), 3.22 (s, 3H), 3.04 (s, 3H), 2.23 (s, 3H), 1.84-1.74 (m, 6H), 1.66-1.60 (m, 1H), 1.50-1.41 (m, 1H), 1.22 (s, 3H). |
| 42 | 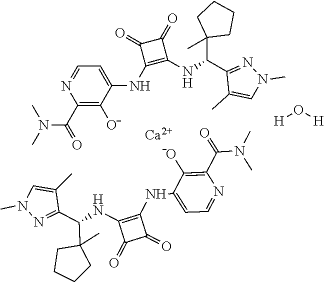 | (R)-4-((2-(((1,4-dimethyl-1H-pyrazol-3-yl)(1- methylcyclopentyl)methyl)amino)-3,4-dioxocyclobut-1- en-1-yl)amino)-3-hydroxy-N,N-dimethylpicolinamide, 0.5 calcium salt, monohydrate; LCMS m/z 467.5 [M + H]⁺. ¹H NMR (400 MHz, DMSO-d₆) δ 9.19 (br. d., J = 8.2 Hz, 1H), 7.66 (d, J = 4.7 Hz, 1H), 7.40 (s, 1H), 7.35 (m, 1H), 5.37 (d, J = 9.8 Hz, 1 H), 3.77 (s, 3H), 3.06 (s, 3H), 2.00 (s, 3H), 1.99 (s, 3H), 1.72- 1.59 (m, 6H), 1.22-1.20 (m, 1H), 1.19-1.18 (m, 1H), 1.09 (s, 3H). |

### CCR6+ T Cell Chemotaxis Assay

Human CD4+CCR6+CXCR3-T cells were isolated from leukopak from healthy donors using EasySep™ Human Th17 Cell Enrichment Kit. (StemCell Technologies, 18162). To obtain large quantities of cells, CCR6+ T cells were activated with Dynabeads Human T-activator (catalog no. 11132D, Gibco) at a density of 1×10⁶ cell/mL in growth media (RPM11640 media with 10% serum, 4 ng/mL IL-2) with a 1:1.5 cell to bead ratio. Day 4 post activation, Dynabeads were removed from the culture. The activated T cells were maintained at 1-2×10⁶ cells/mL for 15 days by feeding fresh growth media when needed.

The CCR6+ T cell chemotaxis assay was carried out on day 12 to 15 post T cell activation using 96 well ChemoTx® Disposable Chemotaxis System (Neuroprobe 101-5) according to the manufacturer's protocol. After one wash with assay buffer (1×HBSS containing 20 mM HEPES and 0.25% BSA), cells were incubated with test compounds for 30 min at room temperature prior to initiation of chemotaxis. For IC₅₀ determination, the top and bottom of the chemotaxis chamber contained the same concentration of compound. DMSO was kept constant at 0.1% (v/v) in all wells. The final concentration of CCL20 (Peprotech 300-29A) in the bottom chamber is 0.5 nM. The fully assembled chemotaxis plate was placed in a cell culture incubator at 37° C., 5% CO₂ for 1 h. After incubation, the top filter was removed, followed by a quick freeze of the bottom chamber at −80° C. for 1 h. The migrated cells in the bottom chamber were stained with CyQUANT dye (Life Technologies, C7026) for cell number determination.

IC₅₀ values for compounds of the present invention (Table 1a) were determined by the non-linear regression analysis of dose response curves.

TABLE 1a

| Example | IC₅₀ (nM) |
|---|---|
| 1 | 5.8 |
| 2 | 186 |
| 3 | 2.3 |
| 4 | 6.3 |
| 5 | 922 |
| 6 | 11.6 |
| 7 | 29.8 |
| 8 | 3.0 |
| 9 | 59.7 |
| 10 | 8.7 |
| 11 | 1.9 |
| 12 | 1.7 |
| 13 | 5.4 |
| 14 | 12.5 |
| 15 | 10.6 |
| 16 | 41.7 |
| 17 | 5.9 |
| 18 | 3.3 |
| 19 | 70.6 |
| 20 | 271 |
| 21 | 102 |
| 22 | 7.4 |
| 23 | 16.5 |
| 24 | 3.1 |
| 25 | 1.2 |
| 26 | 3.3 |
| 27 | 13.2 |
| 28 | 36.7 |
| 29 | 12.0 |
| 30 | 96.1 |
| 31 | 45.4 |
| 32 | 2.7 |
| 33 | 4.4 |
| 34 | 24.0 |
| 35 | 30.0 |
| 36 | 32.3 |
| 37 | 5.2 |
| 38 | 12.6 |
| 39 | 6.5 |
| 40 | ND* |

*No Data

### Human Neutrophil Chemotaxis Assay.

Human neutrophils were purified from freshly drawn human whole blood by immune-magnetic negative selection using the EasySep™ Direct Human Neutrophil Isolation Kit (StemCell Technologies, #19666) according to manufacturer's instructions.

Human neutrophil chemotaxis assay was carried out with Corning FluoroBlok 96 well cell insert system (Corning #351164). Purified neutrophils were resuspended in chemotaxis buffer (1×HBSS containing 25 mM HEPES, pH 7.4 and 0.25% BSA) to a working concentration of 2×10⁶ cells/mL and incubated for 30 min at 37° C. with calcein-AM for cell staining. After 30 min, the labeled neutrophils were washed twice in chemotaxis buffer, resuspended as before and used immediately. For the assay, test compounds were tested in a dose-response format to determine IC₅₀. Labeled neutrophils (50 μL) were pre-incubated with diluted test compound (50 μL) for 30 min in a 96-well plate (Greiner). Next, 100 μL of diluted compound was mixed with 100 μL of GROα (Peprotech #300-11, 2 nM) in the bottom portion of the chemotaxis chamber.

The top portion (insert) of the chemotaxis system containing the FluoroBlok porous membrane was then assembled together. 50 μL of the pre-incubated neutrophils were added to the FluoroBlok insert (top portion) and chemotaxis measured by the neutrophils ability to migrate through the membrane towards the specific CXCR2 ligand, GROα. The Envision multilabel reader (Perkin Elmer) and/or the Typhoon Fluorescent Imager (GE) was used to evaluate changes in fluorescent signal (calcein-AM fluorophore) at 485/535 nm generated by the labeled neutrophils that passed through the porous membrane.

IC₅₀ values for compounds of the present invention (Table 1 b) were determined by the non-linear regression analysis of dose response curves. Navarixin ((R)-2-hydroxy-N,N-dimethyl-3-((2-((1-(5-methylfuran-2-yl)propyl)amino)-3,4-dioxocyclobut-1-en-1-yl)amino)benzamide) was used as a reference control in the assay (IC₅₀=16.8, n=52).

TABLE 1b

| Example | IC₅₀ (nM) |
|---|---|
| 1 | 524 |
| 2 | 7,310 |
| 3 | 1,530 |
| 4 | 886 |
| 5 | ND* |
| 6 | 586 |
| 7 | 2,160 |
| 8 | 103 |
| 9 | 2,750 |
| 10 | 175 |
| 11 | 210 |
| 12 | 107 |
| 13 | 328 |
| 14 | 272 |
| 15 | 735 |
| 16 | 4,350 |
| 17 | 3,160 |
| 18 | 1,010 |
| 19 | 197 |
| 20 | 167 |
| 21 | 293 |
| 22 | 141 |
| 23 | 248 |
| 24 | 1,010 |
| 25 | 355 |
| 26 | 142 |
| 27 | 52.6 |
| 28 | 2,190 |
| 29 | 4,410 |
| 30 | 4,030 |
| 31 | 2,750 |
| 32 | 332 |
| 33 | 69.9 |
| 34 | 2,510 |
| 35 | 255 |
| 36 | 7,130 |
| 37 | 3,630 |
| 38 | 3,460 |
| 39 | 502 |
| 40 | ND* |

*No Data

### Mouse In Vivo Model of IL-23-Induced Psoriasis-Like Skin Inflammation

Efficacy of a CCR6 antagonist of the present invention is evaluated in a mouse in vivo model of psoriasis-like skin inflammation. Recombinant mouse IL-23 is administered via intradermal injections into the left ears of female 8-10 weeks old C57BL/6 mice. This leads to increased psoriasis-like disease symptoms including thickening and erythema in the skin of the left ears. Mice given IL-23 are treated from Day 0 to Day 11 with either daily (QD) oral doses of a CCR6 antagonist or twice daily (BID) doses of a CCR6 antagonist. Additionally, a group of mice are treated with an anti-IL-17 Ab as a positive efficacy control. Representative dosing groups for the study consist of the following:
a group dosed orally twice daily (BID) with vehicle, negative control group,
a group dosed by intraperitoneal injection twice weekly with an anti-IL-17 Ab (5 mg/kg), positive control group,
a group dosed orally with Compound A at 3-100 mg/kg either once a day (QD) or twice a day (BID),
an untreated, naive control group.

Ten mice are enrolled per treatment group with the exception of the naïve control group, which contains five mice. Study day 0 is designated as the first treatment day, and ear thickness measurements are taken daily by an engineer's micrometer (Mitutoyo, Aurora, IL, USA). Ear swelling measurements for each mouse was compared to baseline and expressed in microns.

The compounds of the present invention selectively inhibit T cell chemotaxis compared to neutrophil chemotaxis (see Table 1c). This selectivity renders the compounds of the present invention as viable therapeutic agents for treating inflammatory, immune, autoimmune, neurodegenerative and neuroinflammatory diseases, conditions, or disorders in humans with reduced risk of neutropenia.

TABLE 1c

| | T Cell |
|---|---|
| | Chemotaxis |
| Example | Selectivity |
| 1 | 90 |
| 2 | 39 |
| 3 | 665 |
| 4 | 140 |
| 5 | ND* |
| 6 | 51 |
| 7 | 72 |
| 8 | 34 |
| 9 | 46 |
| 10 | 20 |
| 11 | 110 |
| 12 | 63 |
| 13 | 60 |
| 14 | 22 |
| 15 | 69 |
| 16 | 104 |
| 17 | 535 |
| 18 | 306 |
| 19 | 3 |
| 20 | NS** |
| 21 | 3 |
| 22 | 19 |
| 23 | 15 |
| 24 | 325 |
| 25 | 296 |
| 26 | 43 |
| 27 | 4 |
| 28 | 60 |
| 29 | 367 |
| 30 | 42 |
| 31 | 60 |
| 32 | 123 |
| 33 | 16 |
| 34 | 104 |
| 35 | 8 |
| 36 | 220 |
| 37 | 698 |
| 38 | 274 |
| 39 | 77 |
| 40 | ND* |

*No Data
**Not selective for T cell chemotaxis compared to neutrophil chemotaxis

T cell chemotaxis inhibition versus neutrophil chemotaxis inhibition was unexpectedly enhanced by replacing hydrogen with a methyl group alpha to the point of attachment of the heteroaryl “A” ring (see Table 1d).

TABLE 1d

| | R = H, IC₅₀ (nM) | | | R = CH₃, IC₅₀ (nM) | | |
|---|---|---|---|---|---|---|
| | CCR6 | CXCR2 | | CCR6 | | |
| | T cell, | Neutrophil, | T Cell | T cell, | CXCR2 | |
| | CCL20 | GRO□ | Chemotax | CCL20 | Neutrophil, | T Cell |
| Compound of the | Chemotax | Chemotax | is | Chemotax | GRO□ | Chemotaxis |
| Present Invention | is | is | Selectivity | is | Chemotaxis | Selectivity |
| 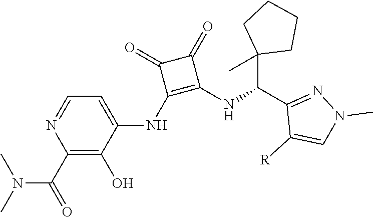 | 19.2 | 42.1 | 2.2 | 5.8 | 524 | 90.3 |
| 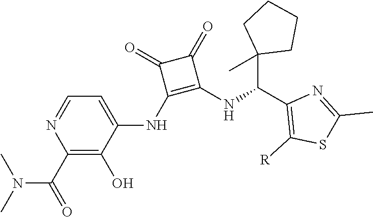 | 32.1 | 346 | 10.8 | 2.7 | 332 | 123 |
| 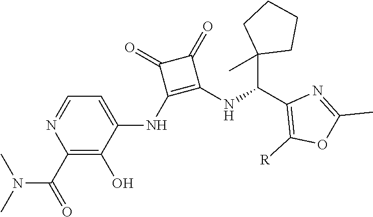 | 64.5 | 12.2 | NS* | 24.0 | 2510 | 104 |

*Not selective for T cell chemotaxis compared to neutrophil chemotaxis

### Co-Precipitation of Compounds of the Disclosure

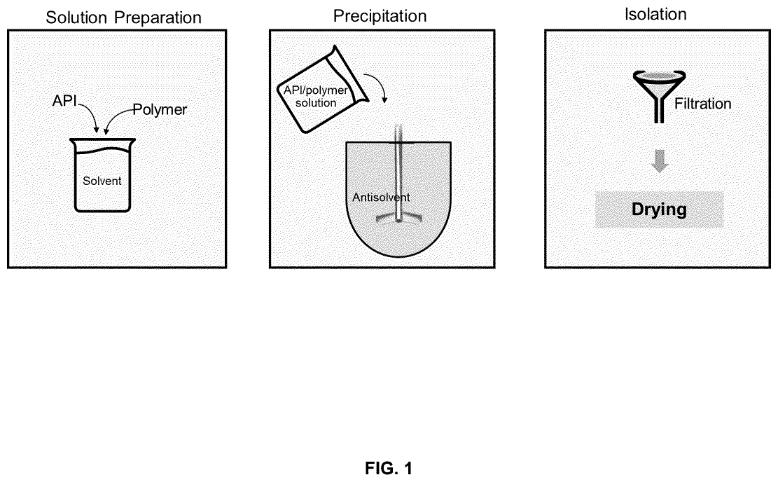

Co-precipitation is a method of generating an amorphous solid dispersion (ASD) of an active pharmaceutical ingredient (API) that demonstrates thermal instability and/or limited solubility, for example, a compound of the disclosure. Changes in processing conditions during co-precipitation can have a significant impact on the properties of the co-precipitated amorphous dispersions. Co-precipitation allows for a wider selection of solvents, can potentially reduce the carbon footprint of preparing a drug product, and allows for larger batch sizes than does spray dry dispersion. Further, APIs prepared with co-precipitation have low bulk density and have porous/irregular shape particles, but tablets prepared with co-precipitated material demonstrate high thermal stability and may have slower disintegration of the API. FIG. 1 illustrates the process of the co-precipitation process of an active pharmaceutical ingredient (API). First, a solution comprising the API and a polymer is prepared. The solution is added to an antisolvent, and the resulting product is filtered and dried to produce a co-precipitated ASD comprising the API.

### Co-Precipitation of Compound A

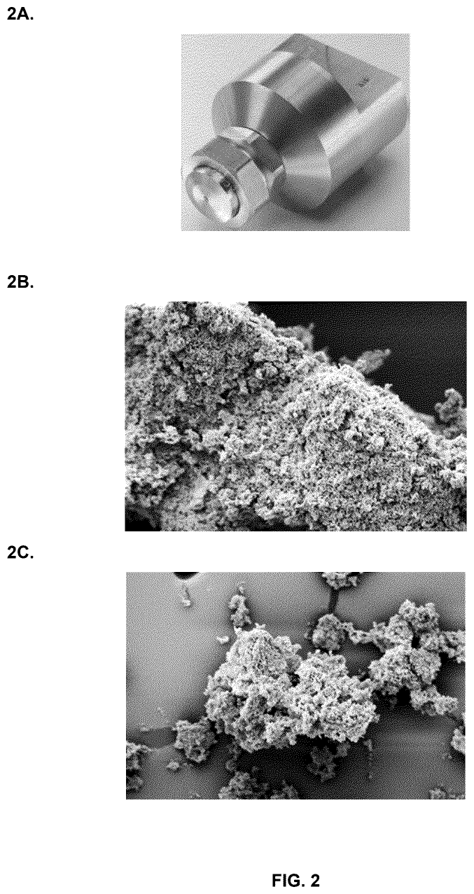

KL1 batch: A first batch of co-precipitated formulation comprising Compound A was prepared using 25% of compound A and 75% hydroxypropyl methylcellulose acetate succinate (M grade) (HPMCAS-M). A spray nozzle was used to generate uniform droplets of the compound A-HPMCAS-M solution before adding the mixture to the antisolvent. FIG. 2A shows an image of the spray nozzle used to prepare a co-precipitation formulation of Compound A (KL1 batch). FIG. 2B and FIG. 2C show images of the co-precipitated material, which was porous with a very high surface area.

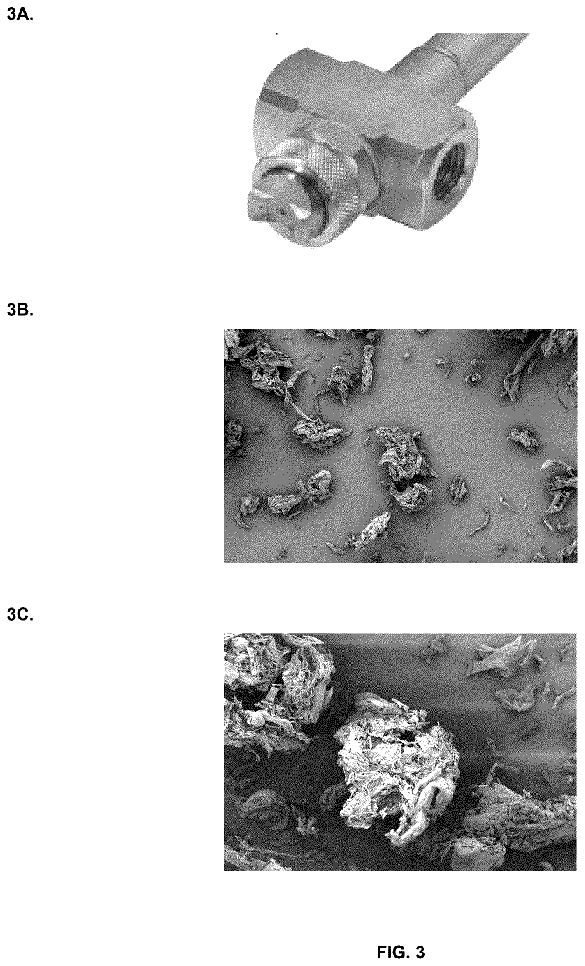

KL2 batch: A second batch of co-precipitated formulation comprising Compound A was prepared using 25% of compound A and 75% (HPMCAS-M). A modified spray nozzle was used to generate uniform droplets of Compound A-HPMCAS-M solution before adding the mixture to the antisolvent. FIG. 3A shows an image of the air-assisted spray nozzle used to prepare a co-precipitation formulation of Compound A (KL2 batch). FIG. 3B and FIG. 3C show images of the co-precipitated material, which had a desired porosity and low surface area.

The co-precipitated materials were characterized by powder X-ray diffraction (PXRD), Thermogravimetric analysis (TGA), Modulated differential scanning calorimetry (mDSC) and Dynamic vapor sorption (DVS). By PXRD analysis, the materials were amorphous, and TGA suggested approximately 0.3% weight loss up to 150° C. mDSC results showed the glass transition temperature of the material was about 116° C. using a heat-cool-heat method. A DVS study revealed an about 7.5% weight uptake at 90% relative humidity, 25° C.

Characterization methods Powder X-ray diffraction (PXRD): Powder X-ray diffraction analysis was conducted using a Bruker AXS D8 Advance diffractometer equipped with a Cu radiation source. Diffracted radiation was detected by a LYNXEYE_EX detector with motorized slits. The divergence slit was set at 10 mm continuous illumination. The secondary slit was set at 5.50 mm. The X-ray tube voltage and amperage were set at 40 kV and 40 mA respectively. Data was collected in the Theta-Theta goniometer in a locked couple scan at Cu K-alpha (average) wavelength from 3.0 to 40.0 degrees 2-Theta with an increment of 0.02 degrees, using a scan speed of 0.5 seconds per step with an anti-scatter screen in place. Samples were rotated at 15/min during collection.

Samples were prepared by placement in a silicon low background sample holder. Data were collected using Bruker DIFFRAC Plus software. Analysis performed by EVA diffract plus software. The sample holder used in a particular experiment is given by a codename within the filename: DW=Deep well holder, SD=small divot holder and FP=Flat plate holder.

Thermogravimetric analysis (TGA): Thermogravimetric analysis was conducted using a TGA 5500 (TA instruments) thermogravimetric analyzer. A sample of approximately 10 mg was weighed into an aluminum pan and heated from ambient temperature to 300° C. at a 10° C./minute heating rate under a nitrogen purge (25 mL/min for sample chamber and 10 mL/min for balance).

Modulated differential scanning calorimetry (mDSC): Modulated differential scanning calorimetry measurements were performed with a Discovery DSC 2500 (TA instruments) equipped with a refrigerated cooling accessory. All experiments were performed in Tzero aluminum pans. The cell constant was determined using indium, and temperature calibration was performed using indium and tin as standards. All measurements were done under a continuous dry nitrogen purge (50 mL/min). Approximately 1-5 mg of solid sample was weighed into a Tzero aluminum pan and sealed non-hermetically. The program was heated from −50° C. to 180° C. using a modulate temperature amplitude of ±1.0° C., a modulation period of 100s, and a ramp rate of 2° C./min, then cooled down to −50° C., followed the 2^{nd} heating from −50° C. to 200° C. using a modulate temperature amplitude of ±1.0° C., a modulation period of 100s, and a ramp rate of 2° C./min. The experimental data were analyzed using commercially available software (TA Universal Analysis 2000/Trios software, TA Instruments).

Dynamic vapor sorption (DVS): Water sorption and desorption studies were conducted on an automated vapor sorption analyzer (TA instruments Q5000 SA). The microbalance was calibrated using a 100 mg standard weight. The relative humidity sensor was calibrated at 5.0%, 11.3%, 32.8%, 52.8%, 75.3%, and 84.3% relative humidity (25° C.) using saturated salt solutions. Approximately 10-20 mg of the powder sample was placed in a quartz sample holder and dried at s 3% relative humidity at 25° C. The attainment of equilibrium was assumed when the weight change of the sample was <0.001 wt % in 5 min or by a maximum equilibration time of 120 minutes. The relative humidity was then progressively increased to 90% in increments of 10% followed by a decrease to a final relative humidity of 10% in 10% relative humidity increments. Again, the attainment of equilibrium was assumed when the weight change of the sample was <0.001 wt % in 5 min or by a maximum equilibration time of 120 minutes. The weight gain at each of the % relative humidity steps is based on the weight after the initial drying step.

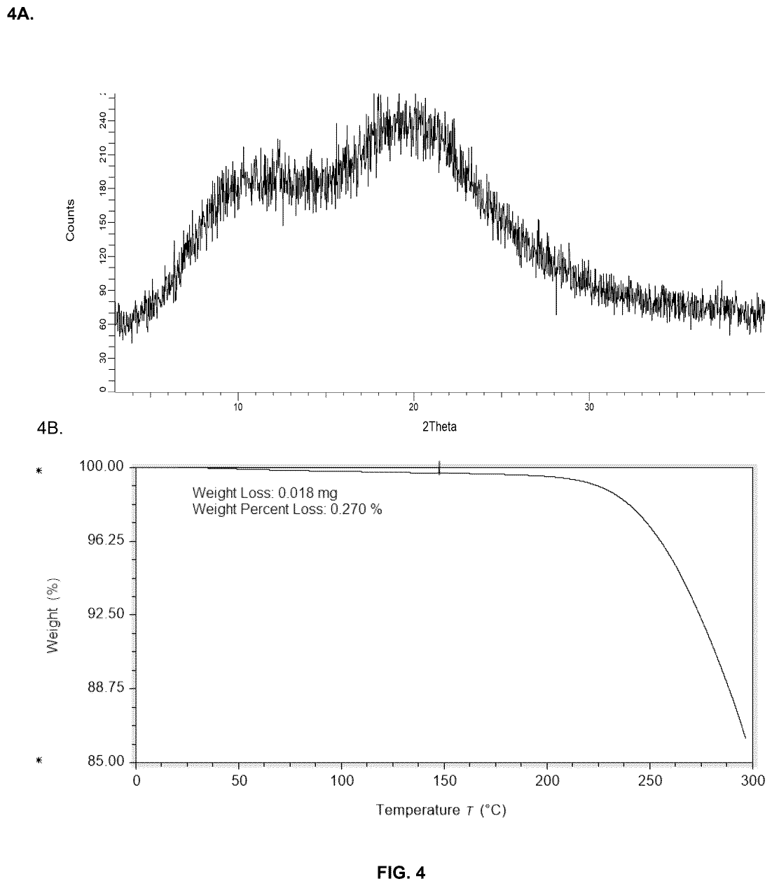

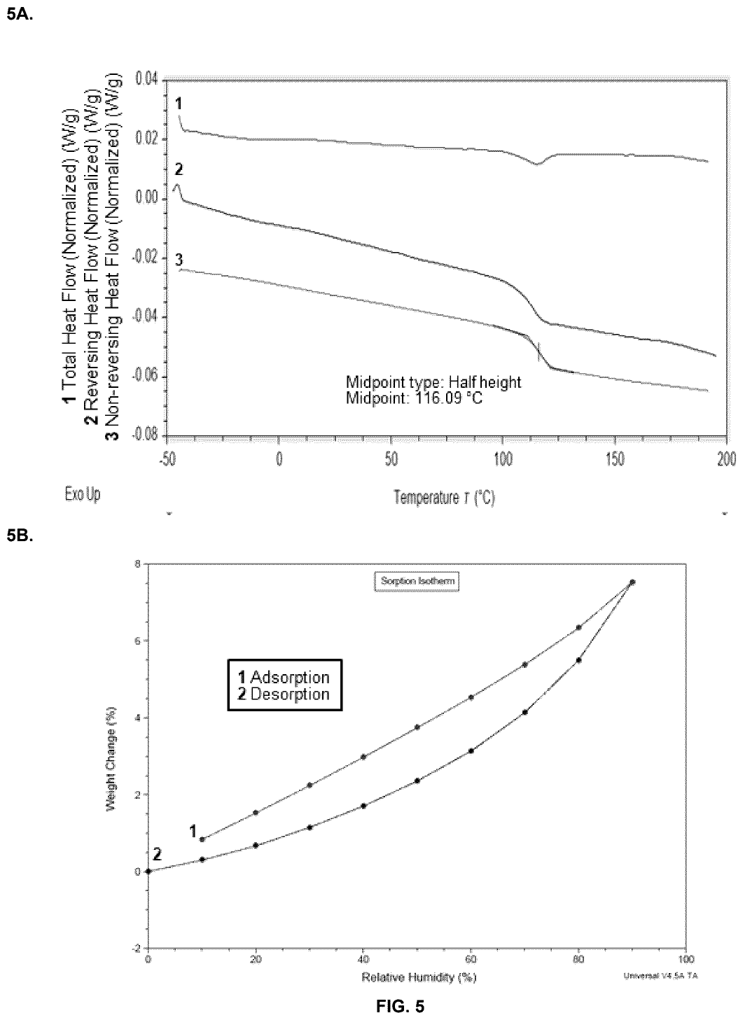

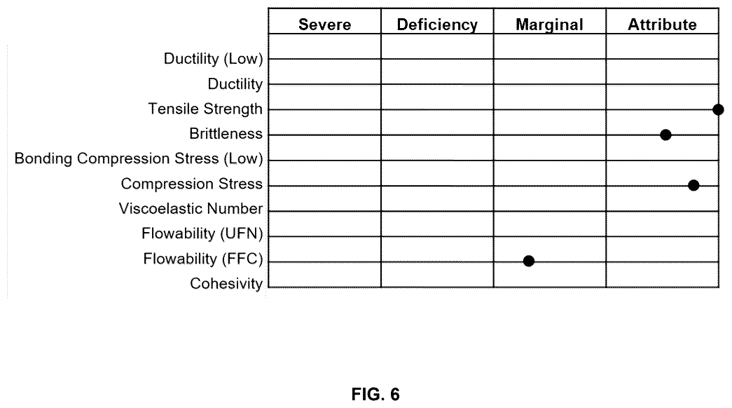

1 kg of a co-precipitation sample was prepared that had a 25 wt % API load. The co-precipitated material was collected with an 87% yield, which included no more than 0.05% THF and no more than 0.32 wt % of heptane. The mechanical properties of the co-precipitated material ranged from moderate to excellent attribute profiles, indicating that the co-precipitated material would have desirable biological activity once formulated. FIG. 4A shows powder X-ray diffraction data obtained for a co-precipitated formulation with Compound A. FIG. 4B shows thermogravimetric analysis data obtained for a co-precipitated formulation with Compound A. FIG. 5A shows modulated differential scanning calorimetry data obtained for a co-precipitated formulation with Compound A. FIG. 5B shows dynamic vapor sorption data obtained for a co-precipitated formulation with Compound A. Table 2 and FIG. 6 show the co-precipitation properties of the material.

TABLE 2
Co-precipitation properties

| Test | Value | Rating |
|---|---|---|
| True density | 1.26 | Acceptable (1-3 g/cc) |
| Bulk Density | 0.091 | Below normal range |
| | | (0.2-0.7 g/cm3) |
| Tapped Density | 0.132 | Below normal range |
| | | (0.3-1.4 g/cm3) |
| Compression stress (Mpa) | 76.9 | Moderate |
| Solid Fraction | 0.85 | |
| Tensile Strength (Mpa) | 9.76 | Very High |
| Compromised Tensile strength | 8.32 | |
| Brittle Fracture index | 0.0863 | Low |
| Flow Function coefficient | 5.90 | Moderate |

### Mechanical and Bulk Properties Measurements

Tensile strength (TS): Square compacts of 0.95 cm×0.95 cm with 0.5 cm thickness were made using a custom-built pneumatic press. Multiple compacts of different solid fractions were prepared. A semi-log linear regression was performed on the measurements, and the values of the parameter was estimated at the reference solid fraction of 0.85. A Large Multi-Function Tester (LMFT) was outfitted with the tensile support frame to measure the tensile strength of a compact. The assembly supported a compact without adding external pressure. The compact was held in a guide in front of a stationary platen held in a solid back support. A platen assembly was attached to the end of the ram. The tensile strength test was performed by pressing the platen into a compact until the compact broke. The tensile strength was calculated using the force at break and the compact thickness as follows:

$${σ}_{T} or {σ}_{{T}_{o}} = \frac{F}{{W}_{p} T} P T F$$

σ_{T}=tensile strength of regular compact
σ_{To}=tensile strength of compromised compact
F=break force
Wₚ=platen width
T=compact thickness
PTF=perpendicular tensile force

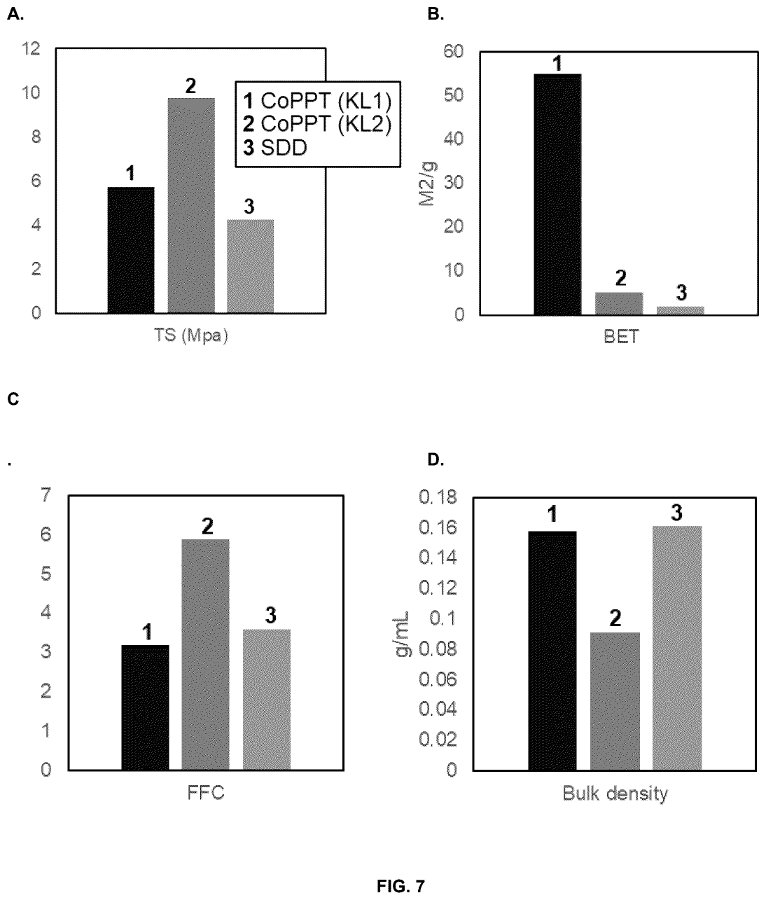

The rate at which force is applied is very important for tensile strength tests because the viscoelastic properties and porosities of specimens affect the tensile strength measurements. Therefore, the stress rate must be normalized through the use of a time constant. The time constant is defined as the time it takes the platen to move from 1/e (0.368) of the break force to the time of tensile failure. The time constant is determined using a regular compact only without a hole. Once the platen rate is adjusted to provide the correct time constant on the regular compact, the same platen rate is then used on compacts with a hole (compromised compacts). A time constant of 15 seconds was used for the co-ppt materials of Compound A. FIG. 7A compares the tensile strength of three different amorphous solid dispersion materials: co-precipitation (KL1), co-precipitation (KL2), and SDD.

Brunauer-Emmett-Teller (BET) surface area for co-precipitated lots: Surface areas of samples were determined on a Micromeritics TriStar II Plus instrument via nitrogen adsorption. Samples were degassed under nitrogen flow at 40° C. for 16 hours on the Micromeritics VacPrep unit prior to the analysis. An 11 point nitrogen isotherm was obtained in the BET region of 0.05-0.30 relative partial pressure, and the BET equation below was used to determine the specific surface area of each sample.

$$\frac{P}{{V}_{a} ( {P}_{0} - P )} = ( \frac{1}{{V}_{m} C} + \frac{C - 1}{{V}_{m} C} ) ( \frac{P}{{P}_{0}} ) \qquad BET Equation$$

Calculated surface-area for spray dried dispersion lots: The surface area for spray dried dispersion samples was calculated based on experimentally determined particle size and true density. The D[3,2] value (surface area weighted mean) was calculated from triplicate laser diffraction measurements conducted on a Sympatec HELOS/L series using an ASPIROS dispersion unit at 1.0 bar. The true density was determined using a Micromeritics AccuPyc II helium pycnometer. The surface area was calculated using the following equation:

$$Surface area ( {m}^{2} \text{/g} ) = \frac{6}{ρ \cdot D [ 3 , 2 ]} .$$

Comparison of surface areas: There was a broad range of surface area between the different preparations of CCR6 ASD. FIG. 7B compares the Brunauer-Emmett-Teller (BET) surface areas of three different amorphous solid dispersion materials: co-precipitation (KL1), co-precipitation (KL2), and SDD. The two co-precipitation lots had a large difference in surface area believed to be the result of differences in manufacturing equipment and process parameters.

Flow function coefficient: Flow function coefficients were measured and calculated using standard ring shear tester method. FIG. 7C shows flow function coefficient measurements for three different solid dispersion materials: co-precipitation (KL1), co-precipitation (KL2), and SDD.

Bulk density: To measure bulk density, the material of interest was weighed and transferred to a graduated cylinder. The weight and volume of the material were recorded. The bulk density was calculated from Bulk density (g/mL)=Weight of material (g)/Volume (mL). FIG. 7D shows bulk density measurements of three different amorphous solid dispersion materials: co-precipitation (KL1), co-precipitation (KL2), and SDD.

### Immediate Release Tablet

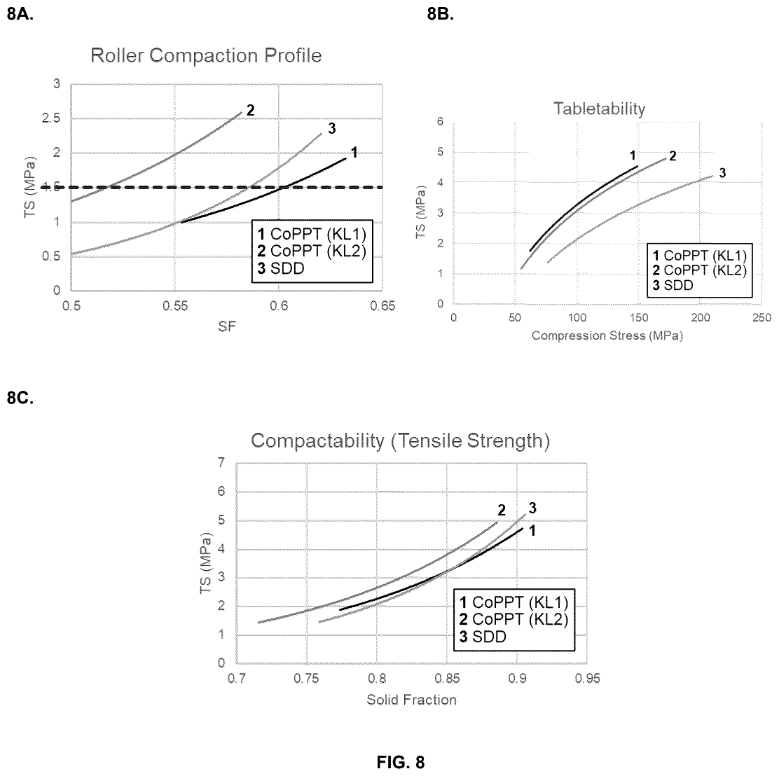

A blend of 40% Co-PPT of Compound A, 3% croscamellose Sodium, 0.5% Mg Stearate was prepared via blend-mill-blend process. The blend was then subjected to roller compaction to make the ribbons at target solid fraction of between 0.5 and 0.65 using StylOne Evo. The ribbons were tested using a split beam tensile tester to create a roller compaction profile shown in FIG. 8A. The compression gap was set to achieve a compact of 2 mm in thickness. After the roller compaction profile was established, the remaining of the blend was compacted to target the solid fraction that achieved a tensile strength of ˜1.5 MPa. The compacts were then passed through a Gerteis minimill. Magnesium stearate was then added to the milled powder and blended. The resulting blend was then compressed using 8 mm round tooling to achieve a target tablet weight of 250 mg. The target solid fraction of the tablets was between 0.70 and 0.9, resulting in the tabletability and compactability profiles shown in FIG. 8B and FIG. 8C.

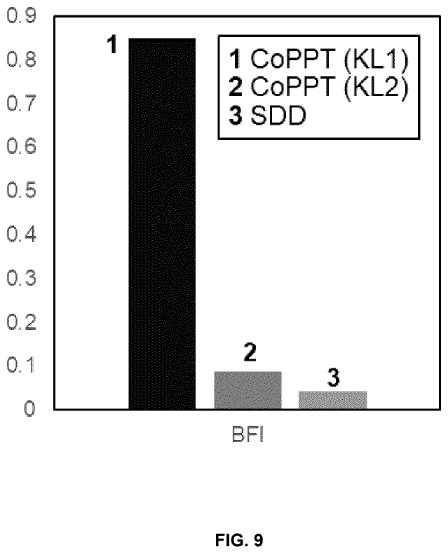

High Brittle Fracture index of co-precipitated tablet: Brittle fracture index is calculated from the equation: BFI=0.5(T/To−1), where T is tensile strength of a rectangular compact made by the process described in the section Tensile strength (TS). To is a tensile strength of a compact with a small hole (defect) described in the section Tensile strength (TS). FIG. 9 shows the brittle fracture index of three different amorphous solid dispersion materials: co-precipitation (KL1), co-precipitation (KL2), and SDD. A higher BFI may indicate capping/lamination tendencies, especially at high compression speed.

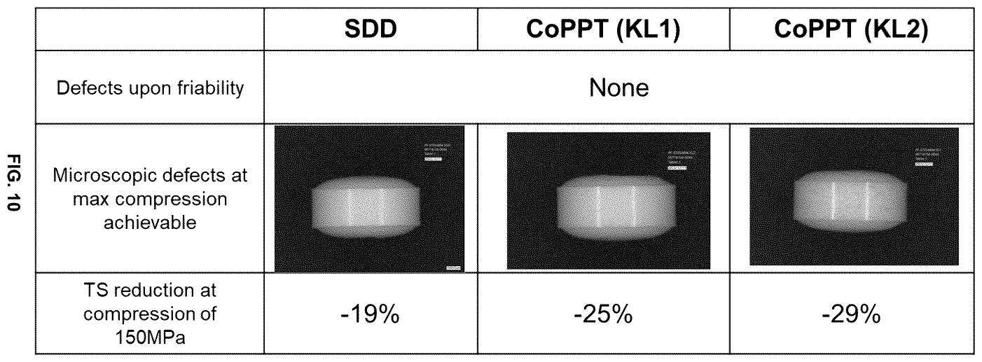

In follow up experiments, tablets of SDD and CoPPT formulations were compressed at higher compression speed. Of 250 k tablets/hr using StylOne Evo. Microscopic images of the tablets were taken at 5× magnification to evaluate the potential micro cracks which were not observed. FIG. 10 shows microscopic images of compressed tablets comprising material made with three different amorphous solid dispersion materials: co-precipitation (KL1), co-precipitation (KL2), and SDD.

### Co-Precipitation and SDD Tablets Dissolution Profiles

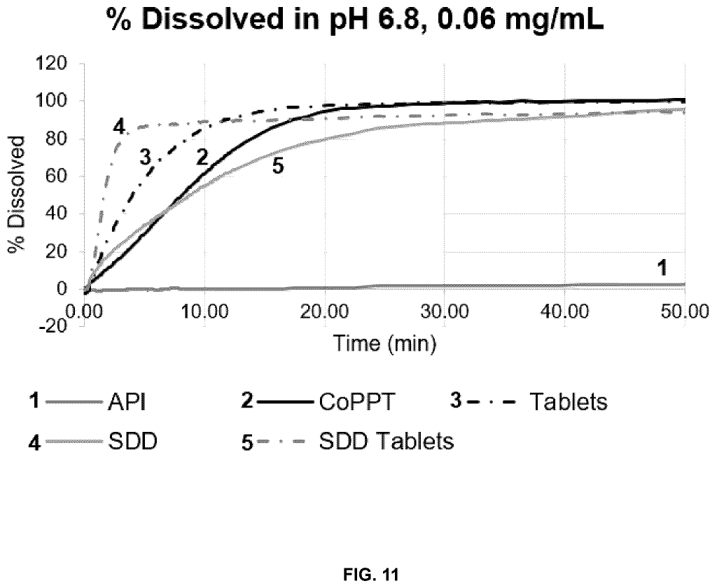

CoPPT and SDD tablets were prepared using the formulation and process outlined above. Dissolution of both the CoPPT and SDD tablets were conducted using a 0.05 M sodium phosphate buffer, pH 6.8 with a maximum release concentration of 0.06 mg/mL. A Pion Rainbow system equipped with six j-probes with a 10 mm path length was used for rapid real time analysis of the dissolution. The dissolution baths were filled with 415 mL of dissolution media, each probe was inserted into a bath and the baths were heated to 37 degrees. Dissolution parameters are as follows: USP apparatus II (paddle), a rotation speed of 50 rpm, 75 minute duration dissolution, infinity spin of 15 minutes starting at 60 minutes with a rotation speed of 150 rpm. The data was exported from the Pion software and into excel and percent dissolved over time was calculated. The CoPPT and CoPPT tablets demonstrated higher supersaturation levels relative to API solubility. The SDD tablets showed slightly faster release than the CoPPT tablets. FIG. 11 shows the dissolution profiles of Compound A, a Co-PPT formulation with compound A, Co-PPT tablet, SDD, and a SDD tablet.

### EMBODIMENTS

The following non-limiting embodiments provide illustrative examples of the invention, but do not limit the scope of the invention.

Embodiment 1. A composition comprising:
a) a compound of Formula (IA) or Formula (IB):

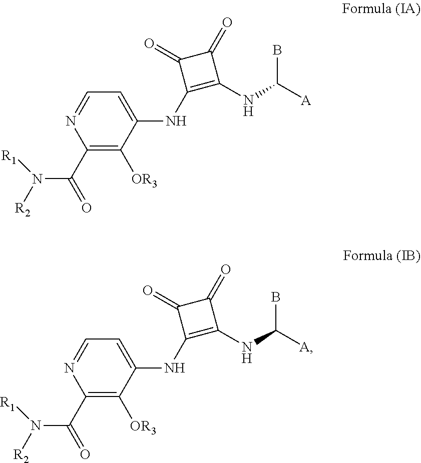

or a pharmaceutically acceptable salt or hydrate thereof, wherein:
R₁ and R₂ are independently H, (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is

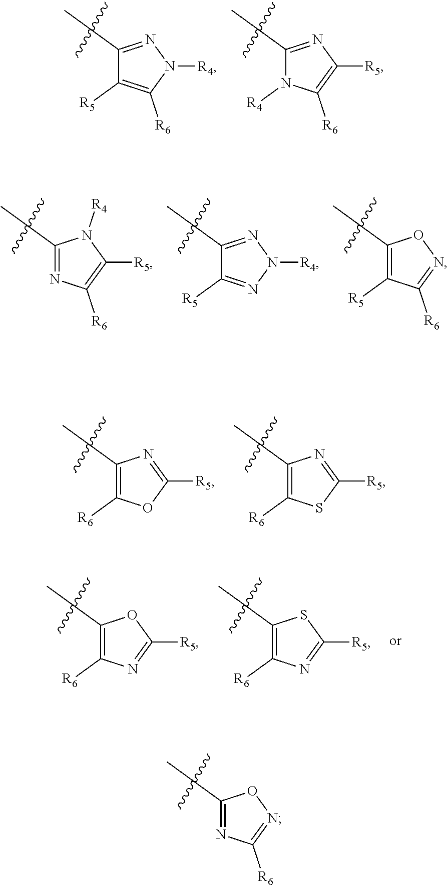

R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁₋₉, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is

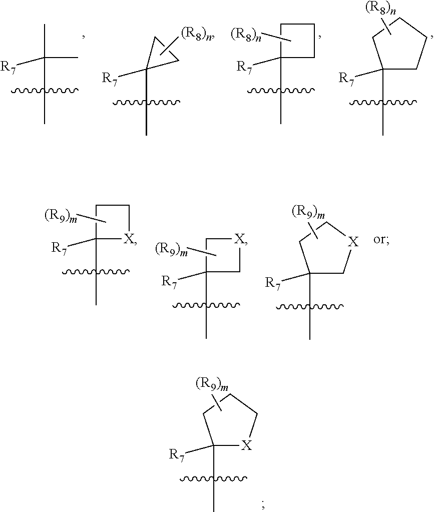

R₇ is —F, —CN, (C₁-C₃)alkyl, (C₁-C₃)alkyl-d₁₋₇, or halo(C₁-C₃)alkyl;
R₈ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₅ at each occurrence is independently deuterium, —F, —Cl, —Br, or —I, or two Res, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl; and
b) a polymer,
wherein the compound and the polymer are in an amorphous solid dispersion.

Embodiment 2. The composition of embodiment 1, wherein the polymer comprises a polyvinyl lactam polymer.

Embodiment 3. The composition of embodiment 2, wherein the polyvinyl lactam polymer is selected from the group consisting of: polyvinylpyrrolidone (PVP), polyvinylpyrrolidone-vinyl acetate copolymer (PVP/VA), and polyvinyl caprolactam-polyvinyl acetate-polyethylene glycol graft copolymer, or a mixture thereof.

Embodiment 4. The composition of embodiment 2, wherein the polyvinyl lactam polymer is polyvinylpyrrolidone (PVP).

Embodiment 5. The composition of any one of embodiments 1-4, wherein the polymer comprises a cellulose derivative polymer.

Embodiment 6. The composition of embodiment 5, wherein the cellulose derivative polymer is selected from the group consisting of: hydroxypropyl cellulose (HPC), hydroxypropyl methylcellulose (HPMC), hydroxyethylcellulose, hydroxypropyl methylcellulose acetate succinate (HPMCAS), hydroxypropyl methylcellulose phthalate (HPMCP), cellulose acetate phthalate (CAP), and polymethyl acrylate, or a mixture thereof.

Embodiment 7. The composition of embodiment 5, wherein the cellulose derivative polymer is hydroxypropyl methylcellulose acetate succinate (HPMCAS).

Embodiment 8. The composition of any one of embodiments 1-7, wherein the composition comprises from about 5% to about 45% of the compound; and from about 55% to about 95% of the polymer.

Embodiment 9. The composition of any one of embodiments 1-7, wherein the composition comprises about 25% of the compound and about 75% of the polymer.

Embodiment 10. The composition of any one of embodiments 1-9, wherein the compound has the Formula (IIA):

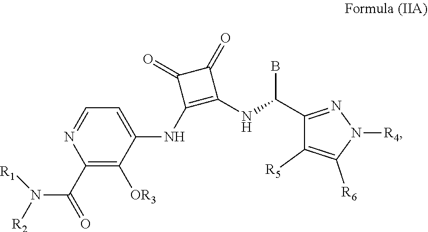

or a pharmaceutically acceptable salt or hydrate thereof, wherein:
R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
R₄ is (C₁-C₄)alkyl;
R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, or halo(C₁-C₄)alkyl;
B is

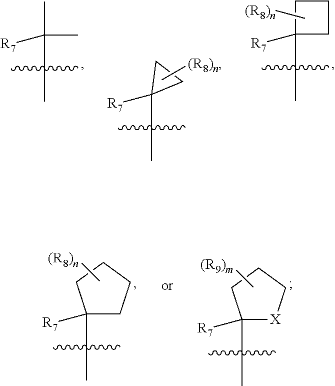

R₇ is (C₁-C₃)alkyl or (C₁-C₃)alkyl-d₁₋₇;
R₈ is deuterium or two R₈s, together with the same carbon atom to which the two R₈s are attached, form a (C₃-C₅)cycloalkyl group;
n is 0 or 2;
R₉ at each occurrence is F;
m is 2; and
X is O.

Embodiment 11. The composition of any one of embodiments 1-10, wherein
R₁ is methyl;
R₂ is methyl, ethyl, isopropyl;
R₃ is H;
A is

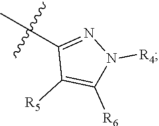

R₄ is methyl;
R₅ is methyl, ethyl, methoxy, Cl, difluoromethoxy, cyano, or cyclopropyl;
R₆ is H;
B is

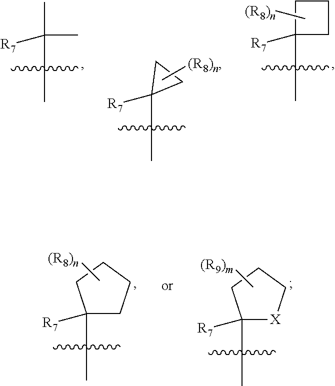

R₇ is methyl;
two R₈s, together with the same carbon atom to which they are attached, form cyclopropyl;
n is 0 or 2;
R₉ at each occurrence is F;
m is 2; and
X is O.

Embodiment 12. The composition of any one of embodiments 1-10, wherein
R₁ is methyl;
R₂ is methyl, ethyl, isopropyl;
R₃ is H;
R₄ is methyl;
R₅ is methyl, ethyl, methoxy, Cl, difluoromethoxy, cyano, or cyclopropyl;
R₆ is H;
B is

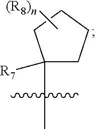

R₇ is methyl; and
n is 0.

Embodiment 13. The composition of embodiment 1, wherein:
R₁ is methyl;
R₂ is methyl, ethyl, or isopropyl;
R₃ is H;
A is

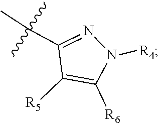

R₄ is methyl;
R₅ is methyl, ethyl, methoxy, Cl, difluoromethoxy, cyano, or cyclopropyl;
R₆ is H;
B is

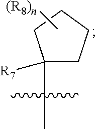

R₇ is methyl; and
n is 0.

Embodiment 14. The composition of any one of embodiments 1-10, wherein:
R₁ and R₂ taken together with the nitrogen atom to which they are attached form morpholine or 4-methylpiperazine;
R₃ is H;
R₄ is methyl;
R₅ is methyl, ethyl, methoxy, Cl, difluoromethoxy, cyano, or cyclopropyl;
R₆ is H;
B is

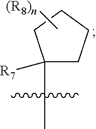

R₇ is methyl; and
n is 0.

Embodiment 15. The composition of any one of embodiments 1-10, wherein the compound is selected from the group consisting of:
(R)-3-(((1,4-dimethyl-1H-pyrazol-3-yl)(1-methylcyclopentyl)methyl)amino)-4-((3-hydroxy-2-(morpholine-4-carbonyl)pyridin-4-yl)amino)cyclobut-3-ene-1,2-dione;
(R)-3-(((1,4-dimethyl-1H-pyrazol-3-yl)(1-methylcyclopentyl)methyl)amino)-4-((3-hydroxy-2-(morpholine-4-carbonyl)pyridin-4-yl)amino)cyclobut-3-ene-1,2-dione;
(R)-3-Hydroxy-N-isopropyl-4-((2-(((4-methoxy-1-methyl-1H-pyrazol-3-yl)(1-methyl-cyclopentyl)methyl)amino)-3,4-dioxocyclobut-1-en-1-yl)amino)-N-methylpicolinamide;
(R)-4-((2-(((2,5-Dimethylthiazol-4-yl)(1-methylcyclopropyl)methyl)amino)-3,4-dioxocyclobut-1-en-1-yl)amino)-3-hydroxy-N,N-dimethylpicolinamide; and
(R)-4-((2-(((2,5-Dimethylthiazol-4-yl)(1-methylcyclopentyl)methyl)amino)-3,4-dioxocyclobut-1-en-1-yl)amino)-3-hydroxy-N,N-dimethylpicolinamide, or a pharmaceutically acceptable salt or hydrate thereof.

Embodiment 16. The composition of any one of embodiments 1-10, wherein the compound is (R)-3-(((1,4-dimethyl-1H-pyrazol-3-yl)(1-methylcyclopentyl)methyl)amino)-4-((3-hydroxy-2-(morpholine-4-carbonyl)pyridin-4-yl)amino)cyclobut-3-ene-1,2-dione, or a pharmaceutically acceptable salt or hydrate thereof.

Embodiment 17. The composition of any one of embodiments 1-10, wherein the compound is (R)-3-Hydroxy-N-isopropyl-4-((2-(((4-methoxy-1-methyl-1H-pyrazol-3-yl)(1-methyl-cyclopentyl)methyl)amino)-3,4-dioxocyclobut-1-en-1-yl)amino)-N-methylpicolinamide, or a pharmaceutically acceptable salt or hydrate thereof.

Embodiment 18. The composition of any one of embodiments 1-10, wherein the compound is (R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, or a pharmaceutically acceptable salt or hydrate thereof.

Embodiment 19. The composition of any one of embodiments 1-10, wherein the compound is (R)-4-((2-(((2,5-Dimethylthiazol-4-yl)(1-methylcyclopropyl)methyl)amino)-3,4-dioxocyclobut-1-en-1-yl)amino)-3-hydroxy-N,N-dimethylpicolinamide, or a pharmaceutically acceptable salt or hydrate thereof.

Embodiment 20. The composition of any one of embodiments 1-10, wherein the compound is (R)-4-((2-(((2,5-Dimethylthiazol-4-yl)(1-methylcyclopentyl)methyl)amino)-3,4-dioxocyclobut-1-en-1-yl)amino)-3-hydroxy-N,N-dimethylpicolinamide, or a pharmaceutically acceptable salt or hydrate thereof.

Embodiment 21. A method of treating a condition comprising administering to a subject in need thereof a pharmaceutical composition comprising:
a) a therapeutically effective amount of a compound of Formula (IA) or Formula (IB):

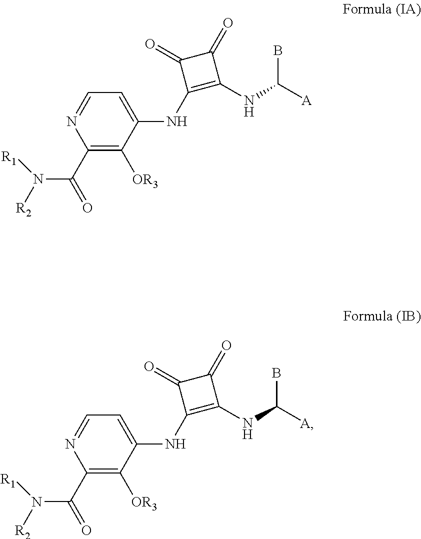

or a pharmaceutically acceptable salt or hydrate thereof, wherein:
R₁ and R₂ are independently H, (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is

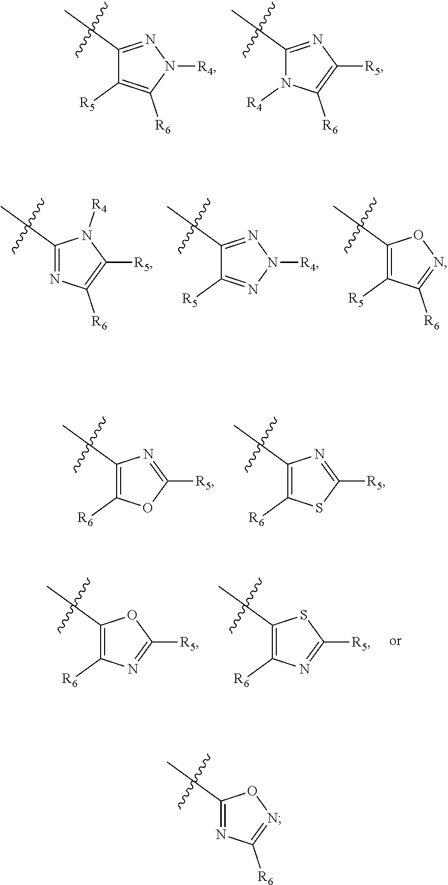

R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁₋₉, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is

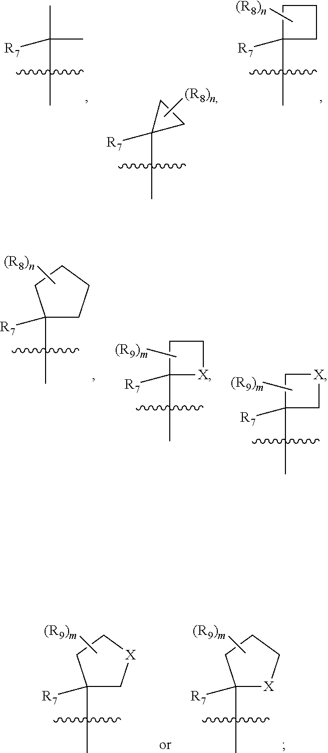

R₇ is —F, —CN, (C₁-C₃)alkyl, (C₁-C₃)alkyl-d₁₋₇, or halo(C₁-C₃)alkyl;
R₈ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₉ at each occurrence is independently deuterium, —F, —Cl, —Br, or —I, or two R₉s, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl; and
b) a polymer,
   wherein the compound and the polymer are in an amorphous solid dispersion,
   wherein the condition is selected from the group consisting of: psoriasis, rheumatoid arthritis, juvenile arthritis, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile idiopathic arthritis (JIA), juvenile psoriatic arthritis, juvenile rheumatoid arthritis, systemic onset juvenile rheumatoid arthritis, giant cell arteritis, secondary osteoarthritis from inflammatory disease, inflammatory bowel disease, Crohn's disease, and ulcerative colitis.

Embodiment 22. The method of embodiment 21, wherein the condition is psoriasis.

Embodiment 23. The method of embodiment 21, wherein the condition is inflammatory bowel disease.

Embodiment 24. The method of embodiment 21, wherein the condition is Crohn's disease.

Embodiment 25. The method of embodiment 21, wherein the condition is ulcerative colitis.

Each of the embodiments described herein may be combined with any other embodiment(s) described herein not inconsistent with the embodiment(s) with which it is combined. In addition, any of the compositions comprising compounds described in the Examples, or pharmaceutically acceptable salts thereof, may be claimed individually or grouped together with one or more other compounds of the Examples, or pharmaceutically acceptable salts thereof, for any of the embodiment(s) described herein. Furthermore, each of the embodiments described herein envisions within its scope pharmaceutically acceptable salts of the compounds described herein.

It will be apparent to those skilled in the art that various modifications and variations may be made in the present invention without departing from the scope or spirit of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

What is claimed is:

1. A composition comprising:
a) a compound of Formula (IA) or Formula (IB): 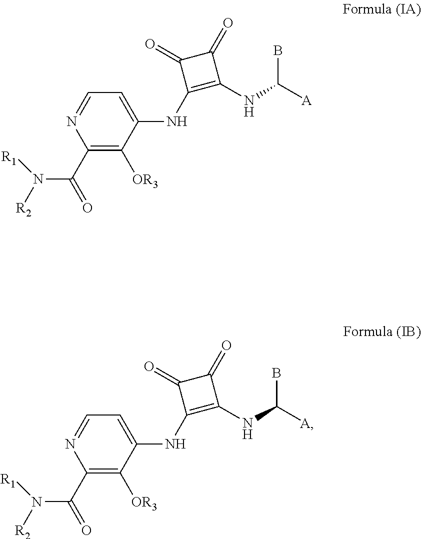
or a pharmaceutically acceptable salt or hydrate thereof, wherein:
R₁ and R₂ are independently H, (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is 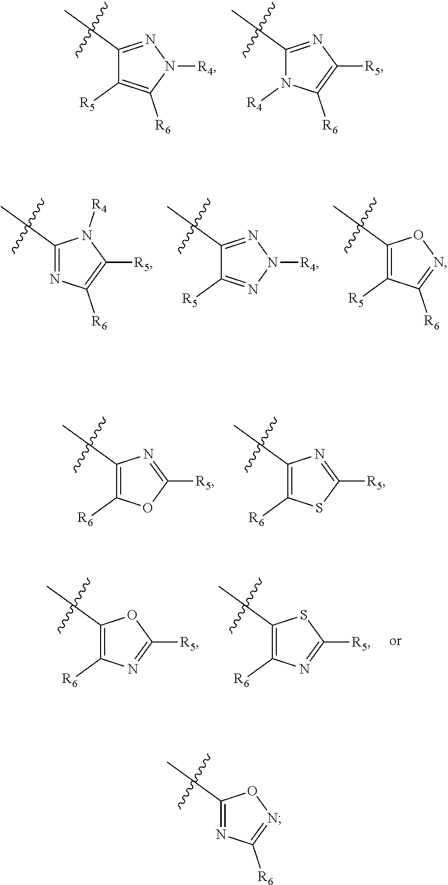
R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁₋₉, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is 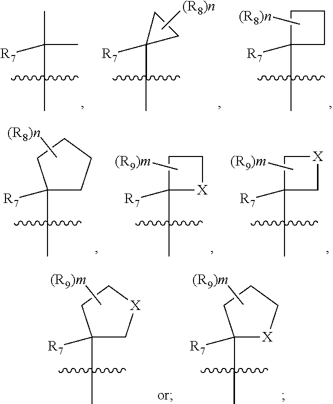
R₇ is —F, —CN, (C₁-C₃)alkyl, (C₁-C₃)alkyl-d₁₋₇, or halo(C₁-C₃)alkyl;
R₃ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₅ at each occurrence is independently deuterium, —F, —Cl, —Br, or —I, or two Res, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl; and
b) a polymer,
wherein the compound and the polymer are in an amorphous solid dispersion.

2. The composition of claim 1, wherein the polymer comprises a polyvinyl lactam polymer.

3. The composition of claim 2, wherein the polyvinyl lactam polymer is selected from the group consisting of: polyvinylpyrrolidone (PVP), polyvinylpyrrolidone-vinyl acetate copolymer (PVP/VA), and polyvinyl caprolactam-polyvinyl acetate-polyethylene glycol graft copolymer, or a mixture thereof.

4. The composition of claim 2, wherein the polyvinyl lactam polymer is polyvinylpyrrolidone (PVP).

5. The composition of claim 1, wherein the polymer comprises a cellulose derivative polymer.

6. The composition of claim 5, wherein the cellulose derivative polymer is selected from the group consisting of: hydroxypropyl cellulose (HPC), hydroxypropyl methylcellulose (HPMC), hydroxyethylcellulose, hydroxypropyl methylcellulose acetate succinate (HPMCAS), hydroxypropyl methylcellulose phthalate (HPMCP), cellulose acetate phthalate (CAP), and polymethyl acrylate, or a mixture thereof.

7. The composition of claim 5, wherein the cellulose derivative polymer is hydroxypropyl methylcellulose acetate succinate (HPMCAS).

8. The composition of claim 1, wherein the composition comprises from about 5% to about 45% of the compound; and from about 55% to about 95% of the polymer.

9. The composition of claim 1, wherein the composition comprises about 25% of the compound and about 75% of the polymer.

10. The composition of claim 1, wherein the compound has the Formula (IIA): 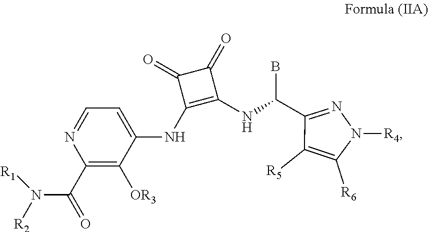
or a pharmaceutically acceptable salt or hydrate thereof, wherein:
R₁ and R₂ are independently (C₁-C₆)alkyl or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, optionally substituted with (C₁-C₄)alkyl;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
R₄ is (C₁-C₄)alkyl;
R₅ and R₆ are independently H, deuterium, (C₁-C₄)alkoxy, (C₁-C₄)alkyl, (C₁-C₄)alkyl-d₁₋₉, (C₃-C₄)cycloalkyl, cyano, halogen, halo(C₁-C₄)alkoxy, or halo(C₁-C₄)alkyl;
B is 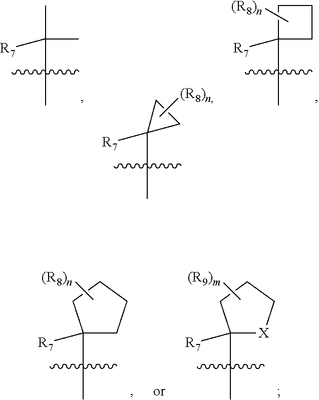
R₇ is (C₁-C₃)alkyl or (C₁-C₃)alkyl-d₁₋₉;
R₈ is deuterium or two R₈s, together with the same carbon atom to which the two R₈s are attached, form a (C₃-C₅)cycloalkyl group;
n is 0 or 2;
R₉ at each occurrence is F;
m is 2; and
X is O.

11. The composition of any claim 10, wherein
R₁ is methyl;
R₂ is methyl, ethyl, isopropyl;
R₃ is H;
A is 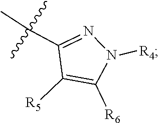
R₄ is methyl;
R₅ is methyl, ethyl, methoxy, Cl, difluoromethoxy, cyano, or cyclopropyl;
R₆ is H;
B is 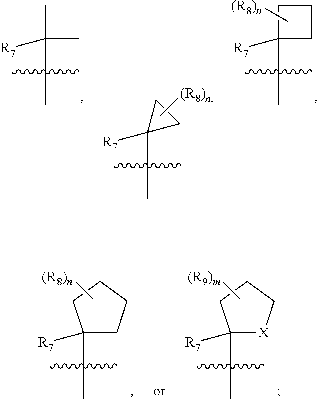
R₇ is methyl;
two R₈s, together with the same carbon atom to which they are attached, form cyclopropyl;
n is 0 or 2;
R₉ at each occurrence is F;
m is 2; and
X is O.

12. The composition of claim 10, wherein
R₁ is methyl;
R₂ is methyl, ethyl, isopropyl;
R₃ is H;
R₄ is methyl;
R₅ is methyl, ethyl, methoxy, Cl, difluoromethoxy, cyano, or cyclopropyl;
R₆ is H;
B is 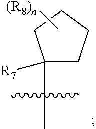
R₇ is methyl; and
n is 0.

13. The composition of claim 1, wherein:
R₁ is methyl;
R₂ is methyl, ethyl, or isopropyl;
R₃ is H;
A is 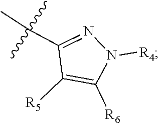
R₄ is methyl;
R₅ is methyl, ethyl, methoxy, Cl, difluoromethoxy, cyano, or cyclopropyl;
R₆ is H;
B is 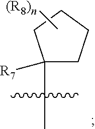
R₇ is methyl; and
n is 0.

14. The composition of claim 1, wherein the compound is selected from the group consisting of:
(R)-3-(((1,4-dimethyl-1H-pyrazol-3-yl)(1-methylcyclopentyl)methyl)amino)-4-((3-hydroxy-2-(morpholine-4-carbonyl)pyridin-4-yl)amino)cyclobut-3-ene-1,2-dione;
(R)-3-(((1,4-dimethyl-1H-pyrazol-3-yl)(1-methylcyclopentyl)methyl)amino)-4-((3-hydroxy-2-(morpholine-4-carbonyl)pyridin-4-yl)amino)cyclobut-3-ene-1,2-dione;
(R)-3-Hydroxy-N-isopropyl-4-((2-(((4-methoxy-1-methyl-1H-pyrazol-3-yl)(1-methyl-cyclopentyl)methyl)amino)-3,4-dioxocyclobut-1-en-1-yl)amino)-N-methylpicolinamide;
(R)-4-((2-(((2,5-Dimethylthiazol-4-yl)(1-methylcyclopropyl)methyl)amino)-3,4-dioxocyclobut-1-en-1-yl)amino)-3-hydroxy-N,N-dimethylpicolinamide;
(R)-4-((2-(((2,5-Dimethylthiazol-4-yl)(1-methylcyclopentyl)methyl)amino)-3,4-dioxocyclobut-1-en-1-yl)amino)-3-hydroxy-N,N-dimethylpicolinamide; and
(R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide;
or a pharmaceutically acceptable salt or hydrate thereof.

15. The composition of claim 1, wherein the compound is (R)-4-((2-(((1,4-Dimethyl-1H-pyrazol-3-yl)(1-methylcyclopentyl)methyl)amino)-3,4-dioxocyclobut-1-en-1-yl)amino)-3-hydroxy-N,N-dimethylpicolinamide, or a pharmaceutically acceptable salt or hydrate thereof.

16. A method of treating a condition comprising administering to a subject in need thereof a pharmaceutical composition comprising:
a) a therapeutically effective amount of a compound of Formula (IA) or Formula (IB): 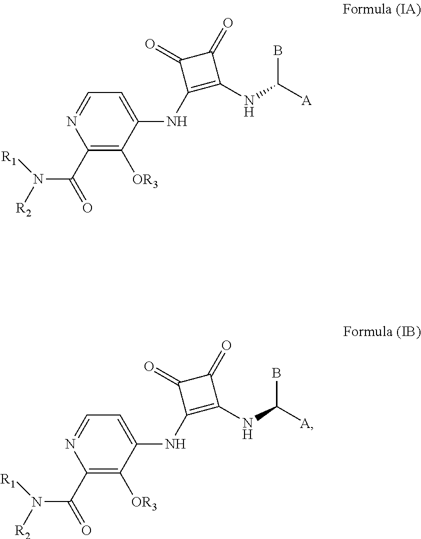
or a pharmaceutically acceptable salt or hydrate thereof, wherein:
R₁ and R₂ are independently H, (C₁-C₆)alkyl, or R₁ and R₂ taken together with the nitrogen atom to which they are attached form a 4-, 5-, or 6-membered heterocycle containing one N heteroatom and optionally 1 or 2 additional heteroatoms selected from the group consisting of O, N, and S, wherein the heterocycle is optionally substituted with 1, 2, or 3 (C₁-C₄)alkyl groups;
R₃ is H, (C₁-C₆)alkyl, (C₁-C₄)alkylcarbonyl, —C(═O)CH═CHCO₂H, —SO₂NH₂, —CH₂OC(═O)(C₁-C₄)alkyl, —CH₂OP(═O)(OH)₂, or —C(═O)NR_{A}R_{B}, wherein the (C₁-C₄)alkylcarbonyl is optionally substituted with —CO₂H or —NH₂, wherein the —CH₂OC(═O)(C₁-C₄)alkyl is optionally substituted with —NH₂, and wherein R_{A} and R_{B} are independently H or (C₁-C₆)alkyl;
A is 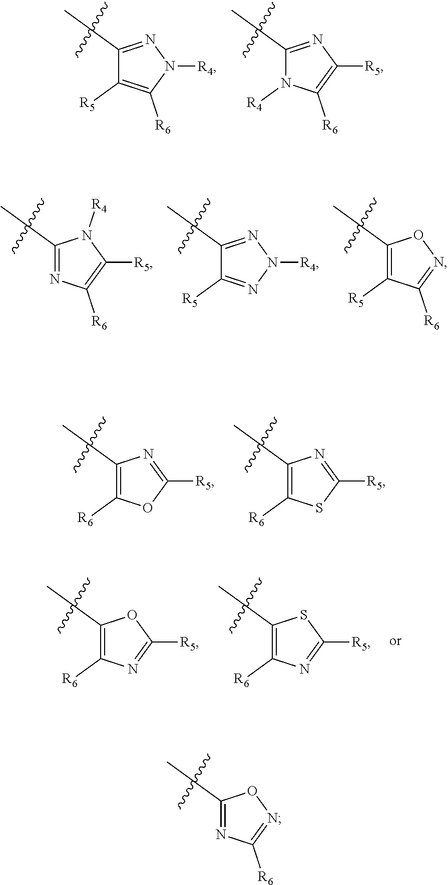
R₄ is H, (C₁-C₄)alkyl, (C₃-C₄)cycloalkyl, (C₃-C₄)cycloalkyl(C₁-C₄)alkyl, or halo(C₁-C₄)alkyl;
R₅ and R₆ are independently H, deuterium, (C₂-C₄)alkenyl, (C₁-C₄)alkoxy, (C₁-C₄)alkyl, (C₁-C₄)alkyl-d₁₋₉, (C₃-C₄)cycloalkyl, (C₃-C₄)cycloalkyl(C₁-C₄)alkyl, cyano, halogen, halo(C₁-C₄)alkoxy, halo(C₁-C₄)alkyl, or hydroxy(C₁-C₄)alkyl;
B is 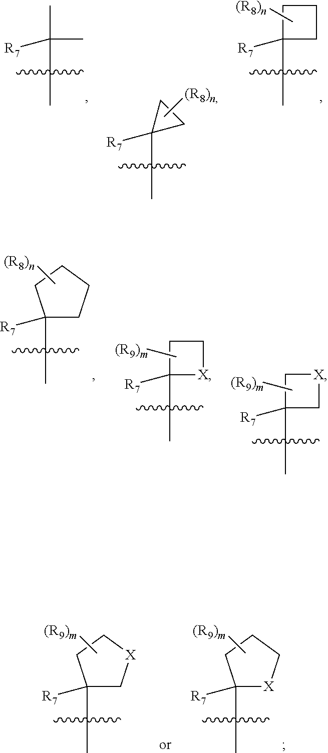
R₇ is —F, —CN, (C₁-C₃)alkyl, (C₁-C₃)alkyl-d₁₋₇, or halo(C₁-C₃)alkyl;
R₃ at each occurrence is independently deuterium, —F, —Cl, —Br, or —I, or two R₈s, together with the same carbon atom to which the two R₈s are attached, form a (C₃-C₅)cycloalkyl group;
n is 0, 1, 2, 3, or 4;
R₅ at each occurrence is independently deuterium, —F, —Cl, —Br, or —I, or two R₉s, together with the same carbon atom to which they are attached, form a (C₃-C₅)cycloalkyl group;
m is 0, 1, 2, 3, or 4; and
X is O, S, or NR_{c}, wherein R_{c} is H or (C₁-C₄)alkyl; and
b) a polymer,
wherein the compound and the polymer are in an amorphous solid dispersion,
wherein the condition is selected from the group consisting of: psoriasis, rheumatoid arthritis, juvenile arthritis, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile idiopathic arthritis (JIA), juvenile psoriatic arthritis, juvenile rheumatoid arthritis, systemic onset juvenile rheumatoid arthritis, giant cell arteritis, secondary osteoarthritis from inflammatory disease, inflammatory bowel disease, Crohn's disease, and ulcerative colitis.

17. The method of claim 16, wherein the condition is psoriasis.

18. The method of claim 16, wherein the condition is inflammatory bowel disease.

19. The method of claim 16, wherein the condition is Crohn's disease.

20. The method of claim 16, wherein the condition is ulcerative colitis.
